(19) United States
(12) Patent Application Publication
(10) Pub. No.: US 2025/0290147 A1
(43) Pub. Date: Sep. 18, 2025

(54) PROSTATE CANCER MARKERS

(71) Applicants: THE INSTITUTE OF CANCER RESEARCH: ROYAL CANCER HOSPITAL, London (GB); CANCER RESEARCH TECHNOLOGY LIMITED, London (GB)

(72) Inventors: Zsofia KOTE-JARAI, London (GB); Daniel BURNS, London (GB); Colin COOPER, London (GB); Rosalind EELES, London (GB)

(21) Appl. No.: 18/860,274

(22) PCT Filed: Apr. 28, 2023
(86) PCT No.: PCT/GB2023/051150

§ 371 Date: Oct. 25, 2024

(30) Foreign Application Priority Data
Apr. 29, 2022 (GB) 2206367.1

(51) Int. Cl. C12Q 1/6886

(52) U.S. Cl. CPC C12Q 1/6886; C12Q 2600/118; C12Q 2600/156; G01N 2800/50

(57) ABSTRACT

A method of predicting a patient's prognosis of prostate cancer, the method comprising providing and analysing a patients germline genetic material and detecting germline variants of genes up-regulated by activation of the PI3K/AKT/mTOR pathway, genes up-regulated by activation of the PI3K/AKT/mTOR pathway, genes up-regulated by KRAS activation, genes up-regulated in response to low oxygen levels, genes regulated by NF-kB in response to tumour necrosis factor (TNF) and/or genes specifically up-regulated in pancreatic beta cells or at least one gene from Table 1. Also provided is a method of determining treatment regimen and a biomarker panel.

The present invention provides methods of predicting the prognosis of subjects who are at risk of or suffer from prostate cancer. The methods include detecting germline variants in a subject's germline genetic material. There is also provided methods of determining a treatment plan based on the subject's prognosis determined by the presence of such germline variants and methods for treatment of prostate cancer. Also provided is a signature biomarker panel suitable for detecting the germline variants.

### BACKGROUND

Prostate cancer (PrCa) is the most common cancer in men in the developed world. Although the majority of PrCa cases are diagnosed with low or intermediate risk disease, approximately 10% of patients develop metastatic disease with poor survival rates [1, 2]. Genetic predisposition to the overall disease risk of PrCa of any severity is well researched; however, understanding of potential heritable genetic factors contributing to tumor progression is limited [3].

Biochemical recurrence (BCR) is often used as a prostate-specific antigen (PSA)-based predictor of progression to poor prognosis phenotype, and is observed in approximately 25% of patients after radical prostatectomy (RP) [4]. Identification of men at high-risk for progression to lethal disease and who are likely to relapse after primary treatment would present the possibility to triage treatment intensification using current or novel systemic therapies. Most research into BCR to date has focused on gene expression or mutational signatures in prostate tumour tissue, or specific candidate genes only [5]. Fewer than 12% of the 241,700 men expected to have been diagnosed with prostate cancer in the United States in 2012 will die from this disease. Many more patients will experience rising prostate-specific antigen (PSA), known as biochemical recurrence (BCR). Physicians treating patients with BCR face a difficult set of decisions in attempting to delay the onset of metastatic disease and death while avoiding over-treating patients whose disease may never affect their overall survival or quality of life. In this generally healthy population, effective management requires that physicians evaluate PSA levels, as well as clinical and radiologic indicators, in order to balance the morbidity and efficacy of proposed treatments against the risks of metastatic progression.

The primary aim of genetic profiling of germline or tumour DNA is to aid clinical decisions, such as targeted screening of asymptomatic individuals and treatment options for cancer patients. Germline signatures in particular would have the advantage of helping to stratify patients in both pre- and post-operative settings. Follow-up strategies and decisions on further treatments could be aided by predicting which individuals are likely to develop prostate tumours, progress to clinically significant disease or relapse.

At least 269 common germline variants (MAF>1%) that explain over a third of the familial relative risk associated with PrCa have been identified [7], but none have been associated exclusively with the aggressive phenotype [7, 8]. Rare germline variants in a small number of genes have however been associated with poor outcome; for example, evidence that BRCA2 is a moderate penetrance gene contributing to young-onset disease with a significantly more aggressive clinical course [9-11], and more recently support for PALB2 [11] and ATM [12]. In addition, loss-of-function (LoF) mutations in a small number of additional DNA repair genes (NBN, and genes associated with Lynch syndrome) have been shown to predispose to familial PrCa and some are associated with more aggressive phenotypes including metastatic disease [13-15].

US2013149703A1 relates to the effects of allelic variants of SRD5A1 and SRD5A2 genes and haplotype-tagging single nucleotide polymorphisms (htSNPs; n=19) on recurrence-free survival after RP and shows that germline polymorphisms in 5α-reductase genes SRD5A1 and SRD5A2 are independent prognostic genetic biomarkers that predict PCa biochemical recurrence after radical prostatectomy and may represent useful molecular tools for a genotype-tailored clinical approach.

There is a need for improved methods of predicting prognosis of prostate cancer patients. There is also a need for improved methods of predicting or determining likelihood of relapse in prostate cancer patients or those at risk of prostate cancer. As such, there is also a need for methods of stratifying prostate cancer patients by risk of relapse as well as methods of determining suitable treatment plans for those at higher risk of relapse.

### BRIEF SUMMARY OF THE DISCLOSURE

The invention is based on the surprising finding that rare germline variants are predictive of poor prognosis after radical treatment. This information can aid clinical management of the disease, particularly at diagnosis, pre- or post-treatment staging and prognostication. It is demonstrated for the first time that rare predicted deleterious coding germline variants robustly associate with time to BCR after radical treatment. The findings show that germline testing at diagnosis could aid clinical decisions by stratifying patients for differential clinical management.

Germline DNA can be sequenced at an early stage of disease or even for healthy individuals which could enable prediction of prostate cancer (PrCa) progression close to, or in advance of, the point of diagnosis. This would allow clinicians to stratify and differentiate patients that are more likely to relapse, putting them on a different clinical treatment plan comprising more radical intervention or more frequent follow-up.

As such, PrCa patients with inherited mutations in specific gene pathways and genes demonstrate a greater likelihood of relapsing after initial radical treatment. Thus, it may be possible to use genetic information to identify sooner which patients may require additional treatments, and in turn improve prognoses for these individuals.

In one aspect of the invention there is provided a method of predicting a patient's prognosis of prostate cancer, the method comprising:
a. providing a sample of the patient's germline genetic material;
b. analysing the patient's germline genetic material;
c. detecting at least one germline variant of at least one gene selected from at least one of;
   genes up-regulated by activation of the PI3K/AKT/mTOR pathway;
   genes defining inflammatory response;
   genes up-regulated by KRAS activation;
   genes up-regulated in response to low oxygen levels;
   genes regulated by NF-kB in response to tumour necrosis factor (TNF); and/or
   genes specifically up-regulated in pancreatic beta cells; or
at least one gene from Table 1;
wherein the prognosis of prostate cancer comprises a characteristic of relapse; and wherein detection of the least one germline variant is predicative of the characteristic of relapse of the prostate cancer patient.

In another aspect of the invention there is provided a method of determining a treatment regimen for a prostate cancer patient, the method comprising;
a. providing a sample of the patient's germline genetic material;
b. analysing the patient's germline genetic material;
c. detecting at least one germline variant of at least one gene selected from at least one of;
   genes up-regulated by activation of the PI3K/AKT/mTOR pathway;
   genes defining inflammatory response;
   genes up-regulated by KRAS activation;
   genes up-regulated in response to low oxygen levels;
   genes regulated by NF-kB in response to tumour necrosis factor (TNF); and/or
   genes specifically up-regulated in pancreatic beta cells; or
at least one gene from Table 1;
d. determining a treatment regimen based on the detection of the at least one germline variant.

In another aspect of the invention there is provided a signature biomarker panel characteristic of time to biochemical relapse and/or likelihood of biochemical relapse for a prostate cancer patient, the panel comprising at least one germline variant of at least one gene selected from at least one of;
genes up-regulated by activation of the PI3K/AKT/mTOR pathway;
genes defining inflammatory response;
genes up-regulated by KRAS activation;
genes up-regulated in response to low oxygen levels;
genes regulated by NF-kB in response to tumour necrosis factor (TNF); and/or
genes specifically up-regulated in pancreatic beta cells; or
at least one gene from Table 1.

In certain embodiments, the characteristic of relapse is time to biochemical relapse (BCR) and/or likelihood of BCR.

In certain embodiments, the patient suffers from prostate cancer or is at risk of prostate cancer.

In certain embodiments, the patient suffers from prostate cancer or has suffered from prostate cancer and has undergone radical therapy.

In certain embodiments, the at least one variant comprises a predicted deleterious mutation.

In certain embodiments, the predicted deleterious mutation comprises a protein-truncating mutation of an encoded protein, and/or wherein the predicted-deleterious variant is a missense variant comprising a CADD PHRED score >30.

In certain embodiments, the protein-truncating mutation comprises one or more of a nonsense, a frameshift and/or a splice site variant.

In certain embodiments, the at least one germline variant comprises a rare variant, optionally wherein the at least one germline variant comprises a minor allele frequency of less than 1%.

In certain embodiments, the least one germline variant comprises a variant of at least one gene selected from at least one of:
the genes of Table 2 (M5923 HALLMARK_PI3K_AKT_MTOR_SIGNALING);
the genes of Table 3 (M5932 HALLMARK_INFLAMMATORY_RESPONSE);
the genes of Table 4 (M5953 HALLMARK_KRAS_SIGNALING_UP);
the genes of Table 5 (M5957 HALLMARK_PANCREAS_BETA_CELLS);
the genes of Table 6 (M5890 HALLMARK_TNFA_SIGNALING_VIA_NFKB); and/or
the genes of Table 7 (M5891 HALLMARK_HYPOXIA).

In certain embodiments, the least one germline variant comprises a variant of at least one of:

PIKFYVE, MYD88, CAB39, RPS6KA1, IRAK2, IL2RB, MSR1, ITGB8, PIK3R5, MMP10, HKDC1, RBM4, GAPDHS, GRHPR, PGM1, SELENBP1, NAGK, SLC6A6, DDX58, KYNU, NR4A1, and/or DENND5A.

In certain embodiments, the least one germline variant comprises a variant of at least one of

PIKFYVE, MYD88, CAB39, RPS6KA1, IRAK2, IL2RB, MSR1, ITGB8, PIK3R5, MMP10, HKDC1 and/or RBM4.

In certain embodiments, detection of the least one germline variant is predicative of the patient's response to a treatment.

In certain embodiments, the characteristic of relapse comprises time to BCR and the least one germline variant comprises a variant of at least one gene selected from:
the genes of Table 2 (M5923 HALLMARK_PI3K_AKT_MTOR_SIGNALING);
the genes of Table 3 (M5932 HALLMARK_INFLAMMATORY_RESPONSE); and/or
the genes of Table 4 (M5953 HALLMARK_KRAS_SIGNALING_UP).
   In certain embodiments, the patient has been diagnosed with a high-grade prostate cancer.

In certain embodiments, the least one germline variant comprises a variant of at least one gene selected from:
the genes of Table 2 (M5923 HALLMARK_PI3K_AKT_MTOR_SIGNALING);
the genes of Table 4 (M5953 HALLMARK_KRAS_SIGNALING_UP);
the genes of Table 5 (M5957 HALLMARK_PANCREAS_BETA_CELLS);
the genes of Table 6 (M5890 HALLMARK_TNFA_SIGNALING_VIA_NFKB); and/or
the genes of Table 7 (M5891 HALLMARK_HYPOXIA).

In certain embodiments, the least one germline variant comprises a variant of at least one of: GAPDHS, GRHPR, PGM1, SELENBP1, NAGK, SLC6A6, PIKFYVE, MYD88, CAB39, RPS6KA1, DDX58, KYNU, NR4A1, DENND5A, MMP10, HKDC1, and/or RBM4.

In certain embodiments, the methods further comprise generating a diagnostic report based on the patient's predicted likelihood and/or time to BCR. In certain embodiments, the diagnostic report is provided to a medical professional (such as a medical doctor) for providing guidance on selection of a prostate cancer treatment to be administered.

In certain embodiments, the methods further comprise administering to the subject a prostate cancer treatment.

In certain embodiments, the methods further comprise administering to the subject a treatment regimen based on the patient's predicted likelihood and/or time to BCR determined by the methods described herein.

In another aspect, the invention provides a method of treating prostate cancer in a patient, the method comprising the steps of administering a prostate cancer treatment wherein the patient has: at least one germline variant of at least one gene selected from at least one of:
genes up-regulated by activation of the PI3K/AKT/mTOR pathway;
genes defining inflammatory response;
genes up-regulated by KRAS activation;
genes up-regulated in response to low oxygen levels;
genes regulated by NF-kB in response to tumour necrosis factor (TNF); and/or
genes specifically up-regulated in pancreatic beta cells; or
at least one gene from Table 1.

In certain embodiments, the patient suffers from prostate cancer and has not undergone therapy and has a predicted increased likelihood of BCR and/or reduced time to BCR the prostate cancer treatment comprises a radical therapy as described herein. For example, prostate cancer treatment comprises radical prostatectomy and/or radical radiotherapy. In some examples, a radial therapy may be administered at a time point earlier than a patient that does not comprise a germline variant as described herein.

In certain embodiments, the patient is at risk of prostate cancer and has a predicted increased likelihood of BCR and/or reduced time to BCR the prostate cancer treatment comprises active surveillance as described herein. For example, initiation of active surveillance or increased active surveillance in comparison to a patient that does not comprise a germline variant as described herein.

In certain embodiments, the patient suffers from prostate cancer or has suffered from prostate cancer and has undergone radical therapy and has a predicted increased likelihood of BCR and/or reduced time to BCR the prostate cancer treatment comprises a further radical therapy. For example, radical chemotherapy.

In certain embodiments, the prostate cancer treatment is selected from the group consisting of:
(i) radical prostatectomy;
(ii) external beam radiotherapy/Brachytherapy (with or without hormone therapy);
(iii) High Intensity Focused Ultrasound (HIFU);
(iv) Cryotherapy;
(v) Trans-urethral resection of the prostate (TURP);
(vi) hormone therapy (e.g. LHRH agonists/GnRH antagonists/Tablets such as Goserelin (Zoladex®), Leuprorelin acetate (Prostap® or Lutrate®), Triptorelin (Decapeptyl® or Gonapeptyl Depot®), Buserelin acetate (Suprefact®), Histrelin (Vantas®), Degarelix (Firmagon®), Bicalutamide (Casodex®), Cyproterone acetate (Cyprostat®), Flutamide (Drogenil®), Abiraterone acetate (Zytiga®), or Nilutamide (Nilandron®))
(vii) Chemotherapy (e.g. Docetaxel (Taxotere®), Cabazitaxel (Jevtana®), Strontium-89 (Metastron®), Samarium-153 (Quadramet®), Enzalutamide (Xtandi®), Radium-223 dichloride (Xofigo®), or Apalutamide (Erleada®))
(viii) Steroids (e.g. Prednisolone, Dexamethasone, Hydrocortisone); and/or
(ix) Sipuleucel-T (Provenge®) (to treat advanced, recurrent prostate cancer).

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows horizontal box plot of the coefficient/log hazard ratios with lower and upper 95% confidence intervals for A) Table 14, B) Table 16 and C) Table 18.

FIG. 2 shows Kaplan-Meier plot showing survival probability against time in months until biochemical recurrence (BCR) for A) all samples, and B) the 336 samples in the high-Gleason subset (Gleason score >3+4; Gleason grade group 3-5). The impact of mutations in significant sets are subdivided by samples with mutations in multiple gene-sets. Log-rank tests for each category: A)=1 (p=0.63); ≥2 (p=2.88×10-3). B)=1 (p=0.27); =2 (p=8.55×10-3); ≥3 (p=3.29×10-3);

FIG. 3 shows an oncoplot of 22 genes from Table 19 altered in 211 of 850 samples. Variants are unfiltered. Right chart shows mutation distribution per gene. Variants annotated as Multi_Hit are those genes which are mutated more than once in the same sample;

FIG. 4 shows an oncoplot of 22 genes from Table 19 altered in 107 of 285 samples with biochemical recurrence. Variants are unfiltered. Right chart shows mutation distribution per gene. Variants annotated as Multi_Hit are those genes which are mutated more than once in the same sample; and

FIG. 5 shows an oncoplot of 22 genes from Table 19 altered in 102 of 565 samples without biochemical recurrence. Variants are unfiltered. Right chart shows mutation distribution per gene. Variants annotated as Multi_Hit are those genes which are mutated more than once in the same sample.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

Provided herein are methods of predicating the prognosis of prostate cancer. The methods provided herein may include stratifying patients. Therefore, the methods may be methods of stratifying patients who suffer from prostate cancer or are at risk of prostate cancer. Stratifying patients based on their prognosis as determined by the methods described herein may also allow for a clinician to determine a differential treatment plan. As such, also provided are methods of determining a treatment plan for a prostate cancer patient based on the detection of germline variants as described herein and methods of treating such subjects.

Thus in one example, there is provided a method of predicting a subject's prognosis of prostate cancer. “Determining prognosis” or “predicting prognosis” refers to methods which can predict the course or outcome of a condition in a subject. The term “prognosis” does not refer to the ability to predict the course or outcome of a condition with 100% accuracy, or even that a given course or outcome is predictably more or less likely to occur based on the detection of germline variants as described herein. Instead, it will be understood that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not having one or more of the germline variants described herein), the chance of a given outcome (e.g., suffering from relapse of prostate cancer) may be very low.

Prognosis may include the likelihood of relapse of subject. The term “relapse” refers to the diagnosis of return, or signs and symptoms of return, of prostate cancer after a period of improvement or remission. “Relapse” can also include “recurrence,” which the National Cancer institute defines as cancer that has recurred, usually after a period of time during which the cancer could not be detected. The cancer may come back to the same location in the body as the original (primary) tumour or to another location in the body (NCI Dictionary of Cancer Terms). In some examples, not detecting a cancer can include not detecting cancer cells in the subject, not detecting tumours in the subject, and/or no symptoms, in whole or in part, associated with the cancer. In some examples, the presence of at least one germline variant as described herein may indicate (i.e. be predictive of) one or more characteristics of relapse. Characteristics of relapse include time to relapse and/or the likelihood of relapse.

In some examples, the relapse is biochemical relapse or recurrence (BCR). For example, prognosis may include time to BCR and/or the likelihood of BCR.

“Biochemical recurrence” or “biochemical relapse” refers, e.g., to recurrent biological values of increased prostate specific antigen (PSA) indicating the presence of prostate cancer cells in a sample. However, it is also possible to use other markers that can be used in the detection of the presence or that raise suspicion of such presence. The rise in the level of prostate specific antigen (PSA) may be at least 0.2 ng/mL in a subject aftertreatment for prostate cancer. Biochemical recurrence may indicate that the prostate cancer has not been treated effectively or has recurred.

PSA is concentrated in prostatic tissue, and serum PSA levels are normally very low. Disruption of the normal prostate architecture, for example by prostatic disease, inflammation or trauma, allows greater amounts of PSA to enter the circulation. PSA is used to detect potential problems in the prostate gland and to follow the progress of prostate cancer therapy.

A blood test to measure PSA is considered the most effective test currently available for the early detection of prostate cancer, although its clinical effectiveness has been questioned. Rising levels of PSA over time are associated with both localized and metastatic prostate cancer. In general, PSA values ranging from 2.5 ng/mL to 4 ng/mL are considered as cut-off values for suspected cancer, and levels above 10 ng/mL indicate higher risk.

The decision to proceed with prostate biopsy is usually made based on results of a PSA assay, which is sometimes also followed by a Digital Rectal Examination (DRE). Results of PSA assay, alone or in combination with results of DRE, are used to select those individuals for prostate biopsy. Further factors may be considered, including free and total PSA, age of the patient, the rate of PSA change with age (PSA velocity), family history, ethnicity, history of prior biopsy, MRI appearance, etc.

Conventional methods of determining PSA may include sending a clinical sample(s) to a commercial laboratory for measurement of PSA levels in a biological fluid sample, or the use of commercially available assay kits for measuring PSA levels in a biological fluid sample. Exemplary kits and suppliers will be apparent to a person of skill in the art. In various examples, PSA may be determined, detected and/or quantified using ELISA assays or lateral flow devices, such as for point-of-care use, as well as spot check colorimetric tests.

Radiation therapy and radical prostatectomy are common treatments for prostate cancer, with over 50% of prostate cancer patients being treated with either or both treatments. However, radiation therapy has a failure rate as high as 25%, and 30-35% of treated prostate cancer patients experience treatment failure within ten years. Predicting BCR prior to treatment or aftertreatment may enable better planning and personalization of treatment. An elevated prostate specific antigen (PSA), for example, 0.2 ng/ml for surgery or 2 ng/ml for radiation therapy above the nadir, is indicative of treatment failure or biochemical recurrence (BCR). BCR is often associated with the presence of more aggressive metastatic prostate cancer and hence worse prognosis.

The method may include or be a method of stratifying patients based on the presence or absence of one or more of the germline variants described herein. The term “stratify” or “stratifying” refers to sorting subjects into those who are more (or less) likely to suffer from relapse as described herein. For example, sorting subjects into strata of those who are more likely (have a higher likelihood) to undergo biochemical reoccurrence (BCR) and/or more likely to have a shorter time to BCR after having undergone radical therapy as described herein and those who are less likely (have a lower likelihood) to undergo biochemical reoccurrence (BCR) and/or more likely to have a longer time to BCR after having undergone radical therapy as described herein wherein the grouping of subjects into these strata is based on detection or absence of one or more of the germline variants described herein.

Based on the detection of one or more of the germline variants described herein, a time to BCR and/or likelihood of BCR may be predicted. For example, a time to BCR and/or likelihood of BCR after radical therapy. For example, detection of one or more of the germline variants described herein may stratify a subject into a group with a reduced or lower time to BCR and/or a greater likelihood of BCR. For example, detection of one or more of the germline variants described herein may stratify a subject into a group with a reduced or lower time to BCR and/or a greater likelihood of BCR after radical therapy. For example, detection of one or more of the germline variants described herein may stratify a subject into a group with a reduced or lower time to BCR in comparison to a subject who does not include one or more of the germline variants described herein. For example, detection of one or more of the germline variants described herein may stratify a subject into a group with a greater or increased likelihood of BCR in comparison to a subject who does not include one or more of the germline variants described herein.

In some examples, detection of one or more of the germline variants described herein may be predictive or an indicator of how a subject may respond to a treatment. For example, how a subject may respond to an initial treatment. For example, the detection of one or more of the germline variants described herein may help to predict how a subject may respond to a radical therapy as an initial therapy.

As described below, detection of one or more of the germline variants described herein may help a clinician determine the most suitable course of therapy for a subject. As such, the methods described herein may further include treating a subject using a therapy selected based on the absence or presence of one or more of the germline variants as described herein.

In some examples, the methods provided herein may be used to predict the likelihood of metastasis of a prostate cancer. BCR has been associated with a significantly increased risk of prostate cancer metastasis (24-34% of patients with BCR will develop metastasis). As such, detection of one or more of the germline variants described herein may be predictive or an indicator of how likely and/or how quickly metastasis may occur in a subject. Therefore, the methods provided herein may also be used to stratify patients by the likelihood or risk of metastasis based on the presence or absence of one or more of the germline variants escribed herein.

### Samples and Analysis

In general, the methods described are in vitro methods that are performed using a sample that includes a subject's germline genetic material that has already been obtained from the subject (i.e. the sample is provided for the method, and the steps taken to obtain the sample from the subject are not included as part of the method).

The methods may therefore include the step of providing a sample from a subject that includes the subject's germline genetic material (i.e. DNA). In particular, a subject's germline genetic material. For example, a subject's genetic material (including both germline genetic material and somatic genetic material) obtained from a sample such as a buccal swab or blood sample may be compared to one or more databases of germline genetic material and/or databases of mutations identified as germline mutations in order to determine and distinguish germline genetic material and somatic genetic material. Example databases include the genome Aggregation Database (gnomAD), the Cancer Genome Atlas (TCGA), the 1000 Genomes Project (1000G) database, Single Nucleotide Polymorphism Database (dbSNP) and NHLBI Exome Variant Server (EVS). In some examples, a subject's germline genetic material may be obtained directly from germ cells such as from a subject's sperm cells or oocyte cells. As such, as used herein the term “germline genetic material” is used to refer to any genetic material from a subject that is determined to be germline material. Therefore, in some examples, methods may include providing a sample of the subject's genetic material and detecting variants therein which have been determined to occur in what has been designated or determined to be germline genetic material.

As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample).

In particular, DNA may be extracted from a non-tumor sample from the subject to be utilized directly for identification of the individual's genetic variations. Particularly, examples of nucleic acid analysis methods are: direct sequencing or pyrosequencing, massively parallel sequencing, high-throughput sequencing (next generation sequencing), high performance liquid chromatography (HPLC) fragment analysis, capillarity electrophoresis and quantitative PCR (as, for example, detection by Taqman® probe, Scorpions™ ARMS Primer or SYBR Green). Several methods for detecting and analyzing PCR amplification products are well known in the art. The general principles and conditions for amplification and detection of genetic variations, such as using PCR, are well known for the skilled person in the art.

Alternatively, other methods of nucleic acid analysis such as hybridization carried out using appropriately labeled probes, detection using microarrays e.g. chips containing many oligonucleotides for hybridization (as, for example, those produced by Affymetrix Corp.) or probe-less technologies and cleavage-based methods may be used. Amplification of DNA can be carried out using primers that are specific to the marker, and the amplified primer extension products can be detected with the use of nucleic acid probes. The DNA may be amplified by PCR prior to incubation with the probe and the amplified primer extension products can be detected using procedure and equipment for detection of the label.

The methods provided herein comprise providing a sample of the subject's germline DNA from blood or saliva samples.

As used herein, the terms “biological sample”, “test sample”, “sample” and variations thereof refer to a sample obtained or derived from a subject. For the purposes described herein, the sample is, or comprises, a biological fluid (also referred to herein as a bodily fluid) sample.

As used herein, the term “biological fluid sample” encompasses a blood sample.

A blood sample may be a whole blood sample, or a processed blood sample e.g. buffy coat. Methods for obtaining biological fluid samples (e.g. whole blood,) from a subject are well known in the art. For example, methods for obtaining blood samples from a subject are well known and include established techniques used in phlebotomy. The obtained blood samples may be further processed using standard techniques. Advantageously, methods for obtaining biological fluid samples from a subject are typically low-invasive or non-invasive.

A whole blood sample is defined as a blood sample drawn from the human body and from which (substantially) no constituents (such as platelets or plasma) have been removed. In other words, the relative ratio of constituents in a whole blood sample is substantially the same as a blood in the body. In this context, “substantially the same” allows for a very small change in the relative ratio of the constituents of whole blood e.g. a change of up to 5%, up to 4%, up to 3%, up to 2%, up to 1% etc. Whole blood contains both the cell and fluid portions of blood. A whole blood sample may therefore also be defined as a blood sample with (substantially) all of its cellular components in plasma, wherein the cellular components (i.e. at least comprising the requisite white blood cells, red blood cells, platelets of blood) are intact.

Therefore, the methods provided herein include analysing a subject's germline genetic material by sequencing. In some examples, sequencing can include whole exome sequencing. In some examples, the sequencing can include whole genome sequencing. In some examples, the sequencing includes sequencing select parts of the genome or exome.

As used herein, the term “exome sequencing” refers to sequencing all protein coding exons of genes in a genome. Exome sequencing can include target enrichment methods such as array-based capture and in-solution capture of nucleic acid, for example. Any sequencing method can be used, including Sanger sequencing using labeled terminators or primers and gel separation in slab or capillary systems, and Next Generation Sequencing (NGS). Exemplary Next Generation Sequencing methodologies include the Roche 454 sequencer, Life Technologies SOLiD systems, the Life Technologies Ion Torrent, and Illumina systems such as the Illumina Genome Analyzer II, Illumina MiSeq, Illumina Hi Seq, and Illumina NovaSeq instruments.

### Variants

The methods described herein predict prognosis, help determine treatment and/or stratify subjects based on detection of germline variants. In some examples, the detection of variants is across the whole genome of a subject. For example, by whole genome sequencing. In some examples, the detection variants may be more targeted, for example by sequencing parts of a subject's genome. For example, selected genes may be sequenced in a targeted panel containing only genes from pathways described here In particular, germline variants in a subject's exome.

A “germline variant” refers to a gene change in a reproductive cell (egg or sperm) that becomes incorporated into the DNA of every cell in the body of the offspring. A variant (or mutation) contained within the germline can be passed from parent to offspring, and is, therefore, hereditary.

In some examples, the germline variants detected in methods of the invention are not somatic variations. As used herein, a “somatic variant” refers to an alteration in DNA that occurs after conception and is not present within the germline. The somatic variant can occur in any of the cells of the body except the germ cells (sperm and egg) and therefore cannot be inherited.

In some examples, the germline variant may be a variant of one or more genes up-regulated by activation of the PI3K/AKT/mTOR pathway. The PI3K/AKT/mTOR pathway plays a crucial role in the regulation of multiple cellular functions including cell growth, proliferation, metabolism and angiogenesis. The PI3K/AKT/mTOR signaling pathway is activated by RTKs (receptor tyrosine kinases), including the insulin receptor (IR), insulin-like growth factor receptor (IGF-1R), platelet-derived growth factor receptor (PDGFR) and epidermal growth factor receptor (EGFR). RTKs can activate PI3K directly or indirectly through insulin receptor substrate (IRS) that interacts with PI3K p85 subunit and further activates PI3K p110 catalytic subunits (Markman et al., (2009) Ann Oncol. 21 (4): 683-91). PI3K is an intracellular phosphatidylinositol kinase. There are three types of PI3K. Class I PI3Ks are mostly cytosolic, are heterodimers comprised of a p110 catalytic subunit and an adaptor/regulatory subunit, and are further divided into two subclasses: Class IA PI3Ks consist of a p110 catalytic subunit that associates with an SH2 domain-containing subunit p85, and is activated by the majority of tyrosine kinase-coupled transmembrane receptors; class IB PI3K consists of a p101 regulatory subunit that associates with p110γ catalytic subunit, and is activated by heterotrimeric GPCR. (Katso et al. (2001) Annu. Rev. Cell Dev. Biol. 17:615). Class II PI3Ks consist of three isoforms. Class III PI3Ks utilize only phosphatidylinositol as a substrate, and play an essential role in protein trafficking through the lysosome. (Volinia, et al. (1995) EMBO J. 14:3339). Class IA PI3K activity is suppressed in cytosol by p85 regulatory subunits that form heterodimers with the p110 catalytic subunit. IRS proteins (including IRS-1, IRS-1, IRS-3, IRS-4) are insulin receptor (IR) and insulin-like growth factor receptor (IGF-1R) adapter proteins. IR/IGF1R activates PI3K by regulating IRS protein tyrosine phosphorylation and subsequent interaction with PI3K p85 subunit. Many cancer tissues overexpress insulin receptor substrate IRS-1, while transgenic overexpression of IRS-1 or IRS-2 in mice caused breast cancer tumorigenesis and metastasis (Metz, et al, (2011) Clin Cancer Res 17: 206-211; Bergmann et al, (1996) Biochem Biophys Res Commun 220: 886-890; Dearth et al, (2006) Mol Cell Biol 26: 9302-9314). Tyrosine phosphorylation of IRS proteins is regulated by IR/IGF-1R and other RTKs such as EGFR and ErbB3 which activate IRS proteins. IRS proteins are also regulated by a number of serine/threonine kinases (for example. PKC, mTOR, S6K and ERK) that phosphorylate IRS proteins on serine leading to protein degradation and inhibition of IRS proteins (Copps et al (2012). Diabetologia. 55(10): 2565-2582). Degradation of insulin receptor substrates by certain drugs results in cell death in melanoma (Reuveni et al (2013) Cancer Res 73: 4383-4394). IRS proteins phosphorylated on tyrosine interact with the SH2 domain of p85 subunit resulting in recruitment of PI3K to membrane and release of the inhibitory effect of p85 leading to activation of PI3K. PI3Ks are enzymes that phosphorylate the 3-hydroxyl position of the inositol ring of phosphoinositides (“PIs”). Activated PI3K generates phosphatidylinositol 3-phosphate (PI3P) that serves as a secondary messenger in growth signaling pathways, influencing cellular events including cell survival, migration, motility, and proliferation; oncogenic transformation; tissue neovascularization; and intracellular protein trafficking. PI3P activates the PI3K-dependent protein kinase-1 (PDK1), which in turn activates the kinase AKT. AKT phosphorylates downstream target molecules to promote cell proliferation, survival and neovascularization. (Cantley et al. (1999) PNAS 96:4240). mTOR is an important signaling molecule downstream of the PI3K/AKT pathway (Grunwald et al. (2002) Cancer Res. 62: 6141; Stolovich et al. (2002) Mol Cell Biol. 22: 8101). AKT-mediated phosphorylation inhibits the GAP activity of TSC1/TSC2 toward the Rheb GTPase, leading to Rheb activation. Rheb binds directly to mTOR, a process that is regulated by amino acids. Both amino acids and Rheb activation are required for mTOR activation. mTOR downstream effector molecules include p70 S6 ribosomal protein kinase (S6K) and eukaryotic initiation factor binding inhibitory protein (4E-BP1). After the activation mTOR phosphorylates and activates the catalytic activity S6K1. mTOR also catalyzes phosphorylation of 4E-BP1 and inactivates it, resulting in initiation of protein translation and cell cycle progression (Kozma et al, (2002) Bioessays 24: 65). More importantly, mTOR exerts a negative feedback on activation of PI3K/AKT by suppressing expression and activation of IRS proteins. Inhibition of mTOR by rapamycin relieves the negative inhibition leading to activation of PI3K AKT (Shi et al (2005) Mol Cancer Ther 2005; 4(10): 1533). Examples of such genes are provided in Tables 1 and 2 below.

In some examples, the germline variant may be a variant of one or more genes defining inflammatory response. Inflammation is one of the highly conserved and beneficial responses evolved in higher organisms in response to pathogens and other harmful stimuli. When a host with a functional innate immune system encounters foreign pathogens or tissue injuries, the inflammatory response initiates. The inflammatory response triggers transcriptional activation of numerous genes, which carry out diverse physiological functions ranging from initiation of antimicrobial activities to the development of acquired immunity. Examples of such genes are provided in Tables 1 and 3 below.

In some examples, the germline variant may be a variant of one or more genes up-regulated by KRAS activation. The KRAS gene provides instructions for making the K-Ras protein that is part of a signalling pathway known as the RAS/MAPK pathway. The protein relays signals from outside the cell to the cell's nucleus. These signals instruct the cell to grow and divide (proliferate) or to mature and take on specialized functions (differentiate). The K-Ras protein is a GTPase, which GTP into another GDP. To transmit signals, K-Ras is bound to a molecule of GTP. The K-Ras protein is turned off (inactivated) when it converts the GTP to GDP. Examples of such genes are provided in Tables 1 and 4 below.

In some examples, the germline variant may be a variant of one or more genes up-regulated in response to low oxygen levels (hypoxia). Cells sense hypoxia and can alter gene expression changing their metabolism in order to promote cell survival. The transcriptional response is mainly mediated by hypoxia-inducible factor 1 (HIF-1) which regulates the transcription of hundreds of genes that promote cell survival in hypoxia. Whether a particular gene is a hypoxia-related gene may be determined by any technique known in the art, including those taught in Lal et al., J. NATL. CANCER INST. (2001) 93:1337-1343; Leonard et al., J. BIOL. CHEM. (2003) 278:40296-40304. For example, cell lines may be grown with the use of standard cell culture techniques either in equilibrium with atmospheric oxygen or in an Environmental Chamber with reduced oxygen designed to approximate the tumour hypoxia levels, see, e.g., Dewhirst et al., RADIAT. RES. (1992) 130:171-182, for hypoxic conditions. Examples of such genes are provided in Tables 1 and 7 below.

In some examples, the germline variant may be a variant of one or more genes regulated by NF-kB in response to tumour necrosis factor (TNF). The NF-κB family of inducible transcription factors is activated in response to a variety of stimuli. Amongst the best-characterized inducers of NF-κB are members of the TNF family of cytokines. NF-κB is a family of inducible transcription factors that play a variety of evolutionarily conserved roles in the immune system. Cytokines belonging to the TNF family induce rapid transcription of genes regulating inflammation, cell survival, proliferation and differentiation, primarily through activation of the NF-κB pathway. The NF-κB family consists of five related proteins, p50 (NF-κB1) and p52 (NF-κB2), p65 (RelA), RelB and c-Rel (Rel), that share an approximately 300 amino acid long N-terminal Rel homology domain (RHD). NF-κB proteins exist in cells as dimers, either homo or heterodimers, that are capable of binding to DNA. The RHD makes direct contact with DNA, while distinct protein domains mediate both positive and negative effects on target gene transcription through the recruitment of co-activators and co-repressors, respectively. The NF-κB proteins p65, c-Rel and RelB possess a transactivation domain allowing them to initiate transcription through co-activator recruitment. The p50 and p52 proteins do not have transactivation domains and therefore can affect transcription either through heterodimerization with p65, c-Rel, or RelB, through competition for binding to κB sites, or through heterotypic interaction with non-Rel transcription factors including certain IκB proteins. Cytokines of the TNF family trigger a variety of NE-KB-dependent responses that can be specific to both cell type and signalling pathway. It is not possible to provide in one article a detailed description of signalling mechanisms triggered by each individual TNF family member. Examples of such genes are provided in Tables 1 and 6 below.

In some examples, the germline variant may be a variant of one or more genes specifically up-regulated in pancreatic beta cells. “genes specifically up-regulated in pancreatic beta cells” refers to any genes whose expression is normally detectable in pancreatic beta cells and associated with insulin expression. In particular, said beta cell genes include the transcription factors BETA2, NKX (6,1 and neurogenin 3, whose expression induces insulin mRNA expression, pro-insulin processing enzymes (prohormone convertase ⅓ and PC2), β-cell protein islet amyloid polypeptide, chromogranin A and synaptogyrin 3. Further examples of such genes are provided in Tables 1 and 5 below.

The germline variants may be a germline variant in any one or more of the genes provided in Table 1 below.

TABLE 1
List of genes that may have variants that are predicative of relapse in
a subject. Genes that occur in multiple pathways are shown in bold.

| | NCBI | Gene |
|---|---|---|
| GENE PATHWAY(S) | GENE ID | Symbol |
| HALLMARK_PI3K_AKT_MTOR_SIGNALING | 31 | ACACA |
| HALLMARK_PI3K_AKT_MTOR_SIGNALING | 10097 | ACTR2 |
| HALLMARK_PI3K_AKT_MTOR_SIGNALING | 10096 | ACTR3 |
| HALLMARK_PI3K_AKT_MTOR_SIGNALING | 108 | ADCY2 |
| HALLMARK_PI3K_AKT_MTOR_SIGNALING | 156 | GRK2 |
| HALLMARK_PI3K_AKT_MTOR_SIGNALING | 207 | AKT1 |
| HALLMARK_PI3K_AKT_MTOR_SIGNALING | 84335 | AKT1S1 |
| HALLMARK_PI3K_AKT_MTOR_SIGNALING | 1173 | AP2M1 |
| HALLMARK_PI3K_AKT_MTOR_SIGNALING | 375 | ARF1 |
| HALLMARK_PI3K_AKT_MTOR_SIGNALING | 396 | ARHGDIA |
| HALLMARK_PI3K_AKT_MTOR_SIGNALING | 10094 | ARPC3 |
| HALLMARK_PI3K_AKT_MTOR_SIGNALING | 466 | ATF1 |
| HALLMARK_PI3K_AKT_MTOR_SIGNALING | 51719 | CAB39 |
| HALLMARK_PI3K_AKT_MTOR_SIGNALING | 811 | CALR |
| HALLMARK_PI3K_AKT_MTOR_SIGNALING | 814 | CAMK4 |
| HALLMARK_PI3K_AKT_MTOR_SIGNALING | 983 | CDK1 |
| HALLMARK_PI3K_AKT_MTOR_SIGNALING | 1017 | CDK2 |
| HALLMARK_PI3K_AKT_MTOR_SIGNALING | 1019 | CDK4 |
| HALLMARK_PI3K_AKT_MTOR_SIGNALING | 1072 | CFL1 |
| HALLMARK_PI3K_AKT_MTOR_SIGNALING | 1213 | CLTC |
| HALLMARK_PI3K_AKT_MTOR_SIGNALING | 1460 | CSNK2B |
| HALLMARK_PI3K_AKT_MTOR_SIGNALING | 27071 | DAPP1 |
| HALLMARK_PI3K_AKT_MTOR_SIGNALING | 1845 | DUSP3 |
| HALLMARK_PI3K_AKT_MTOR_SIGNALING | 1869 | E2F1 |
| HALLMARK_PI3K_AKT_MTOR_SIGNALING | 51295 | ECSIT |
| HALLMARK_PI3K_AKT_MTOR_SIGNALING | 1977 | EIF4E |
| HALLMARK_PI3K_AKT_MTOR_SIGNALING | 356 | FASLG |
| HALLMARK_PI3K_AKT_MTOR_SIGNALING | 8822 | FGF17 |
| HALLMARK_PI3K_AKT_MTOR_SIGNALING | 27006 | FGF22 |
| HALLMARK_PI3K_AKT_MTOR_SIGNALING | 2251 | FGF6 |
| HALLMARK_PI3K_AKT_MTOR_SIGNALING | 9630 | GNA14 |
| HALLMARK_PI3K_AKT_MTOR_SIGNALING | 2792 | GNGT1 |
| HALLMARK_PI3K_AKT_MTOR_SIGNALING | 2885 | GRB2 |
| HALLMARK_PI3K_AKT_MTOR_SIGNALING | 2932 | GSK3B |
| HALLMARK_PI3K_AKT_MTOR_SIGNALING | 3265 | HRAS |
| HALLMARK_PI3K_AKT_MTOR_SIGNALING | 7184 | HSP90B1 |
| HALLMARK_PI3K_AKT_MTOR_SIGNALING | 3565 | IL4 |
| HALLMARK_PI3K_AKT_MTOR_SIGNALING | 51135 | IRAK4 |
| HALLMARK_PI3K_AKT_MTOR_SIGNALING | 3709 | ITPR2 |
| HALLMARK_PI3K_AKT_MTOR_SIGNALING | 5608 | MAP2K6 |
| HALLMARK_PI3K_AKT_MTOR_SIGNALING | 6885 | MAP3K7 |
| HALLMARK_PI3K_AKT_MTOR_SIGNALING | 5594 | MAPK1 |
| HALLMARK_PI3K_AKT_MTOR_SIGNALING | 5602 | MAPK10 |
| HALLMARK_PI3K_AKT_MTOR_SIGNALING | 5599 | MAPK8 |
| HALLMARK_PI3K_AKT_MTOR_SIGNALING | 5601 | MAPK9 |
| HALLMARK_PI3K_AKT_MTOR_SIGNALING | 79109 | MAPKAP1 |
| HALLMARK_PI3K_AKT_MTOR_SIGNALING | 8569 | MKNK1 |
| HALLMARK_PI3K_AKT_MTOR_SIGNALING | 2872 | MKNK2 |
| HALLMARK_PI3K_AKT_MTOR_SIGNALING | 4615 | MYD88 |
| HALLMARK_PI3K_AKT_MTOR_SIGNALING | 4690 | NCK1 |
| HALLMARK_PI3K_AKT_MTOR_SIGNALING | 4793 | NFKBIB |
| HALLMARK_PI3K_AKT_MTOR_SIGNALING | 10392 | NOD1 |
| HALLMARK_PI3K_AKT_MTOR_SIGNALING | 10298 | PAK4 |
| HALLMARK_PI3K_AKT_MTOR_SIGNALING | 5216 | PFN1 |
| HALLMARK_PI3K_AKT_MTOR_SIGNALING | 8503 | PIK3R3 |
| HALLMARK_PI3K_AKT_MTOR_SIGNALING | 200576 | PIKFYVE |
| HALLMARK_PI3K_AKT_MTOR_SIGNALING | 5300 | PIN1 |
| HALLMARK_PI3K_AKT_MTOR_SIGNALING | 5308 | PITX2 |
| HALLMARK_PI3K_AKT_MTOR_SIGNALING | 81579 | PLA2G12A |
| HALLMARK_PI3K_AKT_MTOR_SIGNALING | 23236 | PLCB1 |
| HALLMARK_PI3K_AKT_MTOR_SIGNALING | 5335 | PLCG1 |
| HALLMARK_PI3K_AKT_MTOR_SIGNALING | 5499 | PPP1CA |
| HALLMARK_PI3K_AKT_MTOR_SIGNALING | 5519 | PPP2R1B |
| HALLMARK_PI3K_AKT_MTOR_SIGNALING | 5563 | PRKAA2 |
| HALLMARK_PI3K_AKT_MTOR_SIGNALING | 5571 | PRKAG1 |
| HALLMARK_PI3K_AKT_MTOR_SIGNALING | 5576 | PRKAR2A |
| HALLMARK_PI3K_AKT_MTOR_SIGNALING | 5579 | PRKCB |
| HALLMARK_PI3K_AKT_MTOR_SIGNALING | 5728 | PTEN |
| HALLMARK_PI3K_AKT_MTOR_SIGNALING | 5781 | PTPN11 |
| HALLMARK_PI3K_AKT_MTOR_SIGNALING | 5879 | RAC1 |
| HALLMARK_PI3K_AKT_MTOR_SIGNALING | 5899 | RALB |
| HALLMARK_PI3K_AKT_MTOR_SIGNALING | 8737 | RIPK1 |
| HALLMARK_PI3K_AKT_MTOR_SIGNALING | 6016 | RIT1 |
| HALLMARK_PI3K_AKT_MTOR_SIGNALING | 6195 | RPS6KA1 |
| HALLMARK_PI3K_AKT_MTOR_SIGNALING | 6197 | RPS6KA3 |
| HALLMARK_PI3K_AKT_MTOR_SIGNALING | 57521 | RPTOR |
| HALLMARK_PI3K_AKT_MTOR_SIGNALING | 2810 | SFN |
| HALLMARK_PI3K_AKT_MTOR_SIGNALING | 6503 | SLA |
| HALLMARK_PI3K_AKT_MTOR_SIGNALING | 4087 | SMAD2 |
| HALLMARK_PI3K_AKT_MTOR_SIGNALING | 6773 | STAT2 |
| HALLMARK_PI3K_AKT_MTOR_SIGNALING | 29110 | TBK1 |
| HALLMARK_PI3K_AKT_MTOR_SIGNALING | 117145 | THEM4 |
| HALLMARK_PI3K_AKT_MTOR_SIGNALING | 7074 | TIAM1 |
| HALLMARK_PI3K_AKT_MTOR_SIGNALING | 7132 | TNFRSF1A |
| HALLMARK_PI3K_AKT_MTOR_SIGNALING | 7186 | TRAF2 |
| HALLMARK_PI3K_AKT_MTOR_SIGNALING | 57761 | TRIB3 |
| HALLMARK_PI3K_AKT_MTOR_SIGNALING | 7249 | TSC2 |
| HALLMARK_PI3K_AKT_MTOR_SIGNALING | 7323 | UBE2D3 |
| HALLMARK_PI3K_AKT_MTOR_SIGNALING | 7334 | UBE2N |
| HALLMARK_PI3K_AKT_MTOR_SIGNALING | 10451 | VAV3 |
| HALLMARK_PI3K_AKT_MTOR_SIGNALING | 7529 | YWHAB |
| HALLMARK_{—}PI3K_{—}AKT_{—}MTOR_{—}SIGNALING; | 3932 | LCK |
| HALLMARK_{—}INFLAMMATORY_{—}RESPONSE | | |
| HALLMARK_{—}PI3K_{—}AKT_{—}MTOR_{—}SIGNALING; | 5894 | RAF1 |
| HALLMARK_{—}INFLAMMATORY_{—}RESPONSE | | |
| HALLMARK_{—}PI3K_{—}AKT_{—}MTOR_{—}SIGNALING; | 81617 | CAB39L |
| HALLMARK_{—}KRAS_{—}SIGNALING_{—}UP | | |
| HALLMARK_{—}PI3K_{—}AKT_{—}MTOR_{—}SIGNALING; | 3561 | IL2RG |
| HALLMARK_{—}KRAS_{—}SIGNALING_{—}UP | | |
| HALLMARK_{—}PI3K_{—}AKT_{—}MTOR_{—}SIGNALING; | 4803 | NGF |
| HALLMARK_{—}KRAS_{—}SIGNALING_{—}UP | | |
| HALLMARK_KRAS_SIGNALING_UP | 5243 | ABCB1 |
| HALLMARK_KRAS_SIGNALING_UP | 1636 | ACE |
| HALLMARK_KRAS_SIGNALING_UP | 6868 | ADAM17 |
| HALLMARK_KRAS_SIGNALING_UP | 101 | ADAM8 |
| HALLMARK_KRAS_SIGNALING_UP | 27299 | ADAMDEC1 |
| HALLMARK_KRAS_SIGNALING_UP | 208 | AKT2 |
| HALLMARK_KRAS_SIGNALING_UP | 8854 | ALDH1A2 |
| HALLMARK_KRAS_SIGNALING_UP | 220 | ALDH1A3 |
| HALLMARK_KRAS_SIGNALING_UP | 9949 | AMMECR1 |
| HALLMARK_KRAS_SIGNALING_UP | 56172 | ANKH |
| HALLMARK_KRAS_SIGNALING_UP | 55107 | ANO1 |
| HALLMARK_KRAS_SIGNALING_UP | 11199 | ANXA10 |
| HALLMARK_KRAS_SIGNALING_UP | 347 | APOD |
| HALLMARK_KRAS_SIGNALING_UP | 383 | ARG1 |
| HALLMARK_KRAS_SIGNALING_UP | 83734 | ATG10 |
| HALLMARK_KRAS_SIGNALING_UP | 23080 | AVL9 |
| HALLMARK_KRAS_SIGNALING_UP | 669 | BPGM |
| HALLMARK_KRAS_SIGNALING_UP | 22903 | BTBD3 |
| HALLMARK_KRAS_SIGNALING_UP | 685 | BTC |
| HALLMARK_KRAS_SIGNALING_UP | 760 | CA2 |
| HALLMARK_KRAS_SIGNALING_UP | 867 | CBL |
| HALLMARK_KRAS_SIGNALING_UP | 84869 | CBR4 |
| HALLMARK_KRAS_SIGNALING_UP | 57332 | CBX8 |
| HALLMARK_KRAS_SIGNALING_UP | 894 | CCND2 |
| HALLMARK_KRAS_SIGNALING_UP | 951 | CD37 |
| HALLMARK_KRAS_SIGNALING_UP | 81602 | CDADC1 |
| HALLMARK_KRAS_SIGNALING_UP | 629 | CFB |
| HALLMARK_KRAS_SIGNALING_UP | 3075 | CFH |
| HALLMARK_KRAS_SIGNALING_UP | 3080 | CFHR2 |
| HALLMARK_KRAS_SIGNALING_UP | 1149 | CIDEA |
| HALLMARK_KRAS_SIGNALING_UP | 50856 | CLEC4A |
| HALLMARK_KRAS_SIGNALING_UP | 1363 | CPE |
| HALLMARK_KRAS_SIGNALING_UP | 54677 | CROT |
| HALLMARK_KRAS_SIGNALING_UP | 1438 | CSF2RA |
| HALLMARK_KRAS_SIGNALING_UP | 1520 | CTSS |
| HALLMARK_KRAS_SIGNALING_UP | 23268 | DNMBP |
| HALLMARK_KRAS_SIGNALING_UP | 1794 | DOCK2 |
| HALLMARK_KRAS_SIGNALING_UP | 1848 | DUSP6 |
| HALLMARK_KRAS_SIGNALING_UP | 64123 | ADGRL4 |
| HALLMARK_KRAS_SIGNALING_UP | 2012 | EMP1 |
| HALLMARK_KRAS_SIGNALING_UP | 2022 | ENG |
| HALLMARK_KRAS_SIGNALING_UP | 23136 | EPB41L3 |
| HALLMARK_KRAS_SIGNALING_UP | 2048 | EPHB2 |
| HALLMARK_KRAS_SIGNALING_UP | 2115 | ETV1 |
| HALLMARK_KRAS_SIGNALING_UP | 2118 | ETV4 |
| HALLMARK_KRAS_SIGNALING_UP | 2119 | ETV5 |
| HALLMARK_KRAS_SIGNALING_UP | 7813 | EVI5 |
| HALLMARK_KRAS_SIGNALING_UP | 2162 | F13A1 |
| HALLMARK_KRAS_SIGNALING_UP | 54462 | CCSER2 |
| HALLMARK_KRAS_SIGNALING_UP | 26272 | FBXO4 |
| HALLMARK_KRAS_SIGNALING_UP | 2207 | FCER1G |
| HALLMARK_KRAS_SIGNALING_UP | 2254 | FGF9 |
| HALLMARK_KRAS_SIGNALING_UP | 2324 | FLT4 |
| HALLMARK_KRAS_SIGNALING_UP | 2517 | FUCA1 |
| HALLMARK_KRAS_SIGNALING_UP | 2556 | GABRA3 |
| HALLMARK_KRAS_SIGNALING_UP | 10912 | GADD45G |
| HALLMARK_KRAS_SIGNALING_UP | 2591 | GALNT3 |
| HALLMARK_KRAS_SIGNALING_UP | 2791 | GNG11 |
| HALLMARK_KRAS_SIGNALING_UP | 10457 | GPNMB |
| HALLMARK_KRAS_SIGNALING_UP | 25960 | ADGRA2 |
| HALLMARK_KRAS_SIGNALING_UP | 51704 | GPRC5B |
| HALLMARK_KRAS_SIGNALING_UP | 2982 | GUCY1A1 |
| HALLMARK_KRAS_SIGNALING_UP | 2995 | GYPC |
| HALLMARK_KRAS_SIGNALING_UP | 9734 | HDAC9 |
| HALLMARK_KRAS_SIGNALING_UP | 3018 | H2BC3 |
| HALLMARK_KRAS_SIGNALING_UP | 80201 | HKDC1 |
| HALLMARK_KRAS_SIGNALING_UP | 3237 | HOXD11 |
| HALLMARK_KRAS_SIGNALING_UP | 3290 | HSD11B1 |
| HALLMARK_KRAS_SIGNALING_UP | 3481 | IGF2 |
| HALLMARK_KRAS_SIGNALING_UP | 10320 | IKZF1 |
| HALLMARK_KRAS_SIGNALING_UP | 8808 | IL1RL2 |
| HALLMARK_KRAS_SIGNALING_UP | 90865 | IL33 |
| HALLMARK_KRAS_SIGNALING_UP | 3394 | IRF8 |
| HALLMARK_KRAS_SIGNALING_UP | 3673 | ITGA2 |
| HALLMARK_KRAS_SIGNALING_UP | 3689 | ITGB2 |
| HALLMARK_KRAS_SIGNALING_UP | 9358 | ITGBL1 |
| HALLMARK_KRAS_SIGNALING_UP | 3728 | JUP |
| HALLMARK_KRAS_SIGNALING_UP | 3783 | KCNN4 |
| HALLMARK_KRAS_SIGNALING_UP | 3800 | KIF5C |
| HALLMARK_KRAS_SIGNALING_UP | 7805 | LAPTM5 |
| HALLMARK_KRAS_SIGNALING_UP | 7462 | LAT2 |
| HALLMARK_KRAS_SIGNALING_UP | 3936 | LCP1 |
| HALLMARK_KRAS_SIGNALING_UP | 23643 | LY96 |
| HALLMARK_KRAS_SIGNALING_UP | 7851 | MALL |
| HALLMARK_KRAS_SIGNALING_UP | 11184 | MAP4K1 |
| HALLMARK_KRAS_SIGNALING_UP | 9053 | MAP7 |
| HALLMARK_KRAS_SIGNALING_UP | 23531 | MMD |
| HALLMARK_KRAS_SIGNALING_UP | 4319 | MMP10 |
| HALLMARK_KRAS_SIGNALING_UP | 4320 | MMP11 |
| HALLMARK_KRAS_SIGNALING_UP | 4318 | MMP9 |
| HALLMARK_KRAS_SIGNALING_UP | 10205 | MPZL2 |
| HALLMARK_KRAS_SIGNALING_UP | 54893 | MTMR10 |
| HALLMARK_KRAS_SIGNALING_UP | 4613 | MYCN |
| HALLMARK_KRAS_SIGNALING_UP | 4674 | NAP1L2 |
| HALLMARK_KRAS_SIGNALING_UP | 51199 | NIN |
| HALLMARK_KRAS_SIGNALING_UP | 8431 | NR0B2 |
| HALLMARK_KRAS_SIGNALING_UP | 9971 | NR1H4 |
| HALLMARK_KRAS_SIGNALING_UP | 8829 | NRP1 |
| HALLMARK_KRAS_SIGNALING_UP | 5121 | PCP4 |
| HALLMARK_KRAS_SIGNALING_UP | 27344 | PCSK1N |
| HALLMARK_KRAS_SIGNALING_UP | 80380 | PDCD1LG2 |
| HALLMARK_KRAS_SIGNALING_UP | 5175 | PECAM1 |
| HALLMARK_KRAS_SIGNALING_UP | 5178 | PEG3 |
| HALLMARK_KRAS_SIGNALING_UP | 5284 | PIGR |
| HALLMARK_KRAS_SIGNALING_UP | 5327 | PLAT |
| HALLMARK_KRAS_SIGNALING_UP | 26499 | PLEK2 |
| HALLMARK_KRAS_SIGNALING_UP | 83483 | PLVAP |
| HALLMARK_KRAS_SIGNALING_UP | 5473 | PPBP |
| HALLMARK_KRAS_SIGNALING_UP | 639 | PRDM1 |
| HALLMARK_KRAS_SIGNALING_UP | 5593 | PRKG2 |
| HALLMARK_KRAS_SIGNALING_UP | 5396 | PRRX1 |
| HALLMARK_KRAS_SIGNALING_UP | 5696 | PSMB8 |
| HALLMARK_KRAS_SIGNALING_UP | 58155 | PTBP2 |
| HALLMARK_KRAS_SIGNALING_UP | 79810 | PTCD2 |
| HALLMARK_KRAS_SIGNALING_UP | 5801 | PTPRR |
| HALLMARK_KRAS_SIGNALING_UP | 9910 | RABGAP1L |
| HALLMARK_KRAS_SIGNALING_UP | 5936 | RBM4 |
| HALLMARK_KRAS_SIGNALING_UP | 5950 | RBP4 |
| HALLMARK_KRAS_SIGNALING_UP | 5649 | RELN |
| HALLMARK_KRAS_SIGNALING_UP | 56729 | RETN |
| HALLMARK_KRAS_SIGNALING_UP | 6304 | SATB1 |
| HALLMARK_KRAS_SIGNALING_UP | 29106 | SCG3 |
| HALLMARK_KRAS_SIGNALING_UP | 6447 | SCG5 |
| HALLMARK_KRAS_SIGNALING_UP | 10806 | SDCCAG8 |
| HALLMARK_KRAS_SIGNALING_UP | 7869 | SEMA3B |
| HALLMARK_KRAS_SIGNALING_UP | 12 | SERPINA3 |
| HALLMARK_KRAS_SIGNALING_UP | 51012 | PRELID3B |
| HALLMARK_KRAS_SIGNALING_UP | 6590 | SLPI |
| HALLMARK_KRAS_SIGNALING_UP | 6616 | SNAP25 |
| HALLMARK_KRAS_SIGNALING_UP | 9892 | SNAP91 |
| HALLMARK_KRAS_SIGNALING_UP | 6662 | SOX9 |
| HALLMARK_KRAS_SIGNALING_UP | 8404 | SPARCL1 |
| HALLMARK_KRAS_SIGNALING_UP | 10418 | SPON1 |
| HALLMARK_KRAS_SIGNALING_UP | 6696 | SPP1 |
| HALLMARK_KRAS_SIGNALING_UP | 10253 | SPRY2 |
| HALLMARK_KRAS_SIGNALING_UP | 6480 | ST6GAL1 |
| HALLMARK_KRAS_SIGNALING_UP | 6801 | STRN |
| HALLMARK_KRAS_SIGNALING_UP | 7035 | TFPI |
| HALLMARK_KRAS_SIGNALING_UP | 51311 | TLR8 |
| HALLMARK_KRAS_SIGNALING_UP | 55273 | TMEM100 |
| HALLMARK_KRAS_SIGNALING_UP | 25907 | TMEM158 |
| HALLMARK_KRAS_SIGNALING_UP | 55365 | TMEM176A |
| HALLMARK_KRAS_SIGNALING_UP | 28959 | TMEM176B |
| HALLMARK_KRAS_SIGNALING_UP | 7139 | TNNT2 |
| HALLMARK_KRAS_SIGNALING_UP | 163590 | TOR1AIP2 |
| HALLMARK_KRAS_SIGNALING_UP | 7166 | TPH1 |
| HALLMARK_KRAS_SIGNALING_UP | 28951 | TRIB2 |
| HALLMARK_KRAS_SIGNALING_UP | 10103 | TSPAN1 |
| HALLMARK_KRAS_SIGNALING_UP | 27075 | TSPAN13 |
| HALLMARK_KRAS_SIGNALING_UP | 7102 | TSPAN7 |
| HALLMARK_KRAS_SIGNALING_UP | 10083 | USH1C |
| HALLMARK_KRAS_SIGNALING_UP | 219333 | USP12 |
| HALLMARK_KRAS_SIGNALING_UP | 4013 | VWA5A |
| HALLMARK_KRAS_SIGNALING_UP | 55339 | WDR33 |
| HALLMARK_KRAS_SIGNALING_UP | 7476 | WNT7A |
| HALLMARK_KRAS_SIGNALING_UP | 11179 | ZNF277 |
| HALLMARK_KRAS_SIGNALING_UP | 51193 | ZNF639 |
| HALLMARK_{—}KRAS_{—}SIGNALING_{—}UP; | 205 | AK4 |
| HALLMARK_{—}PANCREAS_{—}BETA_{—}CELLS | | |
| HALLMARK_HYPOXIA | 226 | ALDOA |
| HALLMARK_HYPOXIA | 229 | ALDOB |
| HALLMARK_HYPOXIA | 230 | ALDOC |
| HALLMARK_HYPOXIA | 272 | AMPD3 |
| HALLMARK_HYPOXIA | 55139 | ANKZF1 |
| HALLMARK_HYPOXIA | 302 | ANXA2 |
| HALLMARK_HYPOXIA | 538 | ATP7A |
| HALLMARK_HYPOXIA | 126792 | B3GALT6 |
| HALLMARK_HYPOXIA | 124872 | B4GALNT2 |
| HALLMARK_HYPOXIA | 63827 | BCAN |
| HALLMARK_HYPOXIA | 596 | BCL2 |
| HALLMARK_HYPOXIA | 633 | BGN |
| HALLMARK_HYPOXIA | 665 | BNIP3L |
| HALLMARK_HYPOXIA | 680 | BRS3 |
| HALLMARK_HYPOXIA | 771 | CA12 |
| HALLMARK_HYPOXIA | 839 | CASP6 |
| HALLMARK_HYPOXIA | 857 | CAV1 |
| HALLMARK_HYPOXIA | 901 | CCNG2 |
| HALLMARK_HYPOXIA | 25819 | NOCT |
| HALLMARK_HYPOXIA | 1028 | CDKN1C |
| HALLMARK_HYPOXIA | 9469 | CHST3 |
| HALLMARK_HYPOXIA | 10370 | CITED2 |
| HALLMARK_HYPOXIA | 1289 | COL5A1 |
| HALLMARK_HYPOXIA | 1356 | CP |
| HALLMARK_HYPOXIA | 1466 | CSRP2 |
| HALLMARK_HYPOXIA | 1490 | CCN2 |
| HALLMARK_HYPOXIA | 1634 | DCN |
| HALLMARK_HYPOXIA | 54541 | DDIT4 |
| HALLMARK_HYPOXIA | 10570 | DPYSL4 |
| HALLMARK_HYPOXIA | 1837 | DTNA |
| HALLMARK_HYPOXIA | 1907 | EDN2 |
| HALLMARK_HYPOXIA | 1944 | EFNA3 |
| HALLMARK_HYPOXIA | 2023 | ENO1 |
| HALLMARK_HYPOXIA | 2026 | ENO2 |
| HALLMARK_HYPOXIA | 2027 | ENO3 |
| HALLMARK_HYPOXIA | 54206 | ERRFI1 |
| HALLMARK_HYPOXIA | 2131 | EXT1 |
| HALLMARK_HYPOXIA | 26355 | FAM162A |
| HALLMARK_HYPOXIA | 2203 | FBP1 |
| HALLMARK_HYPOXIA | 2309 | FOXO3 |
| HALLMARK_HYPOXIA | 2548 | GAA |
| HALLMARK_HYPOXIA | 2584 | GALK1 |
| HALLMARK_HYPOXIA | 2597 | GAPDH |
| HALLMARK_HYPOXIA | 26330 | GAPDHS |
| HALLMARK_HYPOXIA | 2632 | GBE1 |
| HALLMARK_HYPOXIA | 2651 | GCNT2 |
| HALLMARK_HYPOXIA | 2817 | GPC1 |
| HALLMARK_HYPOXIA | 2239 | GPC4 |
| HALLMARK_HYPOXIA | 2821 | GPI |
| HALLMARK_HYPOXIA | 9380 | GRHPR |
| HALLMARK_HYPOXIA | 2997 | GYS1 |
| HALLMARK_HYPOXIA | 3036 | HAS1 |
| HALLMARK_HYPOXIA | 3069 | HDLBP |
| HALLMARK_HYPOXIA | 3073 | HEXA |
| HALLMARK_HYPOXIA | 3098 | HK1 |
| HALLMARK_HYPOXIA | 3099 | HK2 |
| HALLMARK_HYPOXIA | 3162 | HMOX1 |
| HALLMARK_HYPOXIA | 3219 | HOXB9 |
| HALLMARK_HYPOXIA | 9957 | HS3ST1 |
| HALLMARK_HYPOXIA | 3309 | HSPA5 |
| HALLMARK_HYPOXIA | 3423 | IDS |
| HALLMARK_HYPOXIA | 3484 | IGFBP1 |
| HALLMARK_HYPOXIA | 10994 | ILVBL |
| HALLMARK_HYPOXIA | 3623 | INHA |
| HALLMARK_HYPOXIA | 3669 | ISG20 |
| HALLMARK_HYPOXIA | 23210 | JMJD6 |
| HALLMARK_HYPOXIA | 11015 | KDELR3 |
| HALLMARK_HYPOXIA | 55818 | KDM3A |
| HALLMARK_HYPOXIA | 3798 | KIF5A |
| HALLMARK_HYPOXIA | 8609 | KLF7 |
| HALLMARK_HYPOXIA | 54800 | KLHL24 |
| HALLMARK_HYPOXIA | 3906 | LALBA |
| HALLMARK_HYPOXIA | 9215 | LARGE1 |
| HALLMARK_HYPOXIA | 3939 | LDHA |
| HALLMARK_HYPOXIA | 3948 | LDHC |
| HALLMARK_HYPOXIA | 4015 | LOX |
| HALLMARK_HYPOXIA | 56925 | LXN |
| HALLMARK_HYPOXIA | 4282 | MIF |
| HALLMARK_HYPOXIA | 4493 | MT1E |
| HALLMARK_HYPOXIA | 4502 | MT2A |
| HALLMARK_HYPOXIA | 4601 | MXI1 |
| HALLMARK_HYPOXIA | 4627 | MYH9 |
| HALLMARK_HYPOXIA | 55577 | NAGK |
| HALLMARK_HYPOXIA | 1463 | NCAN |
| HALLMARK_HYPOXIA | 10397 | NDRG1 |
| HALLMARK_HYPOXIA | 3340 | NDST1 |
| HALLMARK_HYPOXIA | 8509 | NDST2 |
| HALLMARK_HYPOXIA | 23327 | NEDD4L |
| HALLMARK_HYPOXIA | 2908 | NR3C1 |
| HALLMARK_HYPOXIA | 5033 | P4HA1 |
| HALLMARK_HYPOXIA | 8974 | P4HA2 |
| HALLMARK_HYPOXIA | 5066 | PAM |
| HALLMARK_HYPOXIA | 5105 | PCK1 |
| HALLMARK_HYPOXIA | 5155 | PDGFB |
| HALLMARK_HYPOXIA | 5165 | PDK3 |
| HALLMARK_HYPOXIA | 5211 | PFKL |
| HALLMARK_HYPOXIA | 5214 | PFKP |
| HALLMARK_HYPOXIA | 5224 | PGAM2 |
| HALLMARK_HYPOXIA | 5228 | PGF |
| HALLMARK_HYPOXIA | 5230 | PGK1 |
| HALLMARK_HYPOXIA | 5236 | PGM1 |
| HALLMARK_HYPOXIA | 55276 | PGM2 |
| HALLMARK_HYPOXIA | 5260 | PHKG1 |
| HALLMARK_HYPOXIA | 5292 | PIM1 |
| HALLMARK_HYPOXIA | 5317 | PKP1 |
| HALLMARK_HYPOXIA | 51316 | PLAC8 |
| HALLMARK_HYPOXIA | 123 | PLIN2 |
| HALLMARK_HYPOXIA | 10891 | PPARGC1A |
| HALLMARK_HYPOXIA | 8497 | PPFIA4 |
| HALLMARK_HYPOXIA | 5507 | PPP1R3C |
| HALLMARK_HYPOXIA | 25824 | PRDX5 |
| HALLMARK_HYPOXIA | 5578 | PRKCA |
| HALLMARK_HYPOXIA | 112464 | CAVIN3 |
| HALLMARK_HYPOXIA | 284119 | CAVIN1 |
| HALLMARK_HYPOXIA | 5837 | PYGM |
| HALLMARK_HYPOXIA | 3516 | RBPJ |
| HALLMARK_HYPOXIA | 6095 | RORA |
| HALLMARK_HYPOXIA | 58528 | RRAGD |
| HALLMARK_HYPOXIA | 6275 | S100A4 |
| HALLMARK_HYPOXIA | 8819 | SAP30 |
| HALLMARK_HYPOXIA | 949 | SCARB1 |
| HALLMARK_HYPOXIA | 6383 | SDC2 |
| HALLMARK_HYPOXIA | 9672 | SDC3 |
| HALLMARK_HYPOXIA | 8991 | SELENBP1 |
| HALLMARK_HYPOXIA | 6478 | SIAH2 |
| HALLMARK_HYPOXIA | 6576 | SLC25A1 |
| HALLMARK_HYPOXIA | 6518 | SLC2A5 |
| HALLMARK_HYPOXIA | 2542 | SLC37A4 |
| HALLMARK_HYPOXIA | 6533 | SLC6A6 |
| HALLMARK_HYPOXIA | 8406 | SRPX |
| HALLMARK_HYPOXIA | 8987 | STBD1 |
| HALLMARK_HYPOXIA | 6781 | STC1 |
| HALLMARK_HYPOXIA | 8614 | STC2 |
| HALLMARK_HYPOXIA | 6820 | SULT2B1 |
| HALLMARK_HYPOXIA | 26136 | TES |
| HALLMARK_HYPOXIA | 7043 | TGFB3 |
| HALLMARK_HYPOXIA | 7045 | TGFBI |
| HALLMARK_HYPOXIA | 7052 | TGM2 |
| HALLMARK_HYPOXIA | 8277 | TKTL1 |
| HALLMARK_HYPOXIA | 55076 | TMEM45A |
| HALLMARK_HYPOXIA | 7163 | TPD52 |
| HALLMARK_HYPOXIA | 7167 | TPI1 |
| HALLMARK_HYPOXIA | 8459 | TPST2 |
| HALLMARK_HYPOXIA | 7360 | UGP2 |
| HALLMARK_HYPOXIA | 7428 | VHL |
| HALLMARK_HYPOXIA | 7436 | VLDLR |
| HALLMARK_HYPOXIA | 8839 | CCN5 |
| HALLMARK_HYPOXIA | 26118 | WSB1 |
| HALLMARK_HYPOXIA | 7511 | XPNPEP1 |
| HALLMARK_HYPOXIA | 23036 | ZNF292 |
| HALLMARK_HYPOXIA | 133 | ADM |
| HALLMARK_{—}HYPOXIA; | 136 | ADORA2B |
| HALLMARK_{—}INFLAMMATORY_{—}RESPONSE | | |
| HALLMARK_{—}HYPOXIA; | 9435 | CHST2 |
| HALLMARK_{—}INFLAMMATORY_{—}RESPONSE | | |
| HALLMARK_{—}HYPOXIA; | 2719 | GPC3 |
| HALLMARK_{—}INFLAMMATORY_{—}RESPONSE | | |
| HALLMARK_{—}HYPOXIA; | 7162 | TPBG |
| HALLMARK_{—}INFLAMMATORY_{—}RESPONSE | | |
| HALLMARK_{—}HYPOXIA; | 9590 | AKAP12 |
| HALLMARK_{—}INFLAMMATORY_{—}RESPONSE | | |
| HALLMARK_{—}HYPOXIA; | 51129 | ANGPTL4 |
| HALLMARK_{—}KRAS_{—}SIGNALING_{—}UP | | |
| HALLMARK_{—}HYPOXIA; | 30001 | ERO1A |
| HALLMARK_{—}KRAS_{—}SIGNALING_{—}UP | | |
| HALLMARK_{—}HYPOXIA; | 2113 | ETS1 |
| HALLMARK_{—}KRAS_{—}SIGNALING_{—}UP | | |
| HALLMARK_{—}HYPOXIA; | 2745 | GLRX |
| HALLMARK_{—}KRAS_{—}SIGNALING_{—}UP | | |
| HALLMARK_{—}HYPOXIA; | 3486 | IGFBP3 |
| HALLMARK_{—}KRAS_{—}SIGNALING_{—}UP | | |
| HALLMARK_{—}HYPOXIA; | 4214 | MAP3K1 |
| HALLMARK_{—}KRAS_{—}SIGNALING_{—}UP | | |
| HALLMARK_{—}HYPOXIA; | 2645 | GCK |
| HALLMARK_{—}KRAS_{—}SIGNALING_{—}UP | | |
| HALLMARK_{—}HYPOXIA; | 5313 | PKLR |
| HALLMARK_{—}PANCREAS_{—}BETA_{—}CELLS | | |
| HALLMARK_{—}HYPOXIA; | 1027 | CDKN1B |
| HALLMARK_{—}PANCREAS_{—}BETA_{—}CELLS | | |
| HALLMARK_{—}HYPOXIA; | 1649 | DDIT3 |
| HALLMARK_{—}PI3K_{—}AKT_{—}MTOR_{—}SIGNALING | | |
| HALLMARK_{—}HYPOXIA; | 1956 | EGFR |
| HALLMARK_{—}PI3K_{—}AKT_{—}MTOR_{—}SIGNALING | | |
| HALLMARK_{—}HYPOXIA; | 5163 | PDK1 |
| HALLMARK_{—}PI3K_{—}AKT_{—}MTOR_{—}SIGNALING | | |
| HALLMARK_{—}HYPOXIA; | 6513 | SLC2A1 |
| HALLMARK_{—}PI3K_{—}AKT_{—}MTOR_{—}SIGNALING | | |
| HALLMARK_{—}HYPOXIA; | 7852 | CXCR4 |
| HALLMARK_{—}PI3K_{—}AKT_{—}MTOR_{—}SIGNALING | | |
| HALLMARK_{—}HYPOXIA; | 374 | AREG |
| HALLMARK_{—}PI3K_{—}AKT_{—}MTOR_{—}SIGNALING; | | |
| HALLMARK_{—}KRAS_{—}SIGNALING_{—}UP | | |
| HALLMARK_TNFA_SIGNALING_VIA_NFKB | 2683 | B4GALT1 |
| HALLMARK_TNFA_SIGNALING_VIA_NFKB | 9334 | B4GALT5 |
| HALLMARK_TNFA_SIGNALING_VIA_NFKB | 597 | BCL2A1 |
| HALLMARK_TNFA_SIGNALING_VIA_NFKB | 602 | BCL3 |
| HALLMARK_TNFA_SIGNALING_VIA_NFKB | 604 | BCL6 |
| HALLMARK_TNFA_SIGNALING_VIA_NFKB | 329 | BIRC2 |
| HALLMARK_TNFA_SIGNALING_VIA_NFKB | 10950 | BTG3 |
| HALLMARK_TNFA_SIGNALING_VIA_NFKB | 6351 | CCL4 |
| HALLMARK_TNFA_SIGNALING_VIA_NFKB | 595 | CCND1 |
| HALLMARK_TNFA_SIGNALING_VIA_NFKB | 57018 | CCNL1 |
| HALLMARK_TNFA_SIGNALING_VIA_NFKB | 960 | CD44 |
| HALLMARK_TNFA_SIGNALING_VIA_NFKB | 941 | CD80 |
| HALLMARK_TNFA_SIGNALING_VIA_NFKB | 9308 | CD83 |
| HALLMARK_TNFA_SIGNALING_VIA_NFKB | 1051 | CEBPB |
| HALLMARK_TNFA_SIGNALING_VIA_NFKB | 1052 | CEBPD |
| HALLMARK_TNFA_SIGNALING_VIA_NFKB | 8837 | CFLAR |
| HALLMARK_TNFA_SIGNALING_VIA_NFKB | 23529 | CLCF1 |
| HALLMARK_TNFA_SIGNALING_VIA_NFKB | 2919 | CXCL1 |
| HALLMARK_TNFA_SIGNALING_VIA_NFKB | 2920 | CXCL2 |
| HALLMARK_TNFA_SIGNALING_VIA_NFKB | 2921 | CXCL3 |
| HALLMARK_TNFA_SIGNALING_VIA_NFKB | 23586 | DDX58 |
| HALLMARK_TNFA_SIGNALING_VIA_NFKB | 23258 | DENND5A |
| HALLMARK_TNFA_SIGNALING_VIA_NFKB | 11080 | DNAJB4 |
| HALLMARK_TNFA_SIGNALING_VIA_NFKB | 55332 | DRAM1 |
| HALLMARK_TNFA_SIGNALING_VIA_NFKB | 1844 | DUSP2 |
| HALLMARK_TNFA_SIGNALING_VIA_NFKB | 1846 | DUSP4 |
| HALLMARK_TNFA_SIGNALING_VIA_NFKB | 1847 | DUSP5 |
| HALLMARK_TNFA_SIGNALING_VIA_NFKB | 1958 | EGR1 |
| HALLMARK_TNFA_SIGNALING_VIA_NFKB | 1959 | EGR2 |
| HALLMARK_TNFA_SIGNALING_VIA_NFKB | 1960 | EGR3 |
| HALLMARK_TNFA_SIGNALING_VIA_NFKB | 10938 | EHD1 |
| HALLMARK_TNFA_SIGNALING_VIA_NFKB | 10209 | EIF1 |
| HALLMARK_TNFA_SIGNALING_VIA_NFKB | 2114 | ETS2 |
| HALLMARK_TNFA_SIGNALING_VIA_NFKB | 24147 | FJX1 |
| HALLMARK_TNFA_SIGNALING_VIA_NFKB | 2354 | FOSB |
| HALLMARK_TNFA_SIGNALING_VIA_NFKB | 8061 | FOSL1 |
| HALLMARK_TNFA_SIGNALING_VIA_NFKB | 2526 | FUT4 |
| HALLMARK_TNFA_SIGNALING_VIA_NFKB | 1647 | GADD45A |
| HALLMARK_TNFA_SIGNALING_VIA_NFKB | 4616 | GADD45B |
| HALLMARK_TNFA_SIGNALING_VIA_NFKB | 2669 | GEM |
| HALLMARK_TNFA_SIGNALING_VIA_NFKB | 3280 | HES1 |
| HALLMARK_TNFA_SIGNALING_VIA_NFKB | 9592 | IER2 |
| HALLMARK_TNFA_SIGNALING_VIA_NFKB | 51278 | IER5 |
| HALLMARK_TNFA_SIGNALING_VIA_NFKB | 64135 | IFIH1 |
| HALLMARK_TNFA_SIGNALING_VIA_NFKB | 3433 | IFIT2 |
| HALLMARK_TNFA_SIGNALING_VIA_NFKB | 51561 | IL23A |
| HALLMARK_TNFA_SIGNALING_VIA_NFKB | 3572 | IL6ST |
| HALLMARK_TNFA_SIGNALING_VIA_NFKB | 182 | JAG1 |
| HALLMARK_TNFA_SIGNALING_VIA_NFKB | 3726 | JUNB |
| HALLMARK_TNFA_SIGNALING_VIA_NFKB | 23135 | KDM6B |
| HALLMARK_TNFA_SIGNALING_VIA_NFKB | 7071 | KLF10 |
| HALLMARK_TNFA_SIGNALING_VIA_NFKB | 10365 | KLF2 |
| HALLMARK_TNFA_SIGNALING_VIA_NFKB | 687 | KLF9 |
| HALLMARK_TNFA_SIGNALING_VIA_NFKB | 8942 | KYNU |
| HALLMARK_TNFA_SIGNALING_VIA_NFKB | 3914 | LAMB3 |
| HALLMARK_TNFA_SIGNALING_VIA_NFKB | 9516 | LITAF |
| HALLMARK_TNFA_SIGNALING_VIA_NFKB | 1326 | MAP3K8 |
| HALLMARK_TNFA_SIGNALING_VIA_NFKB | 4082 | MARCKS |
| HALLMARK_TNFA_SIGNALING_VIA_NFKB | 4170 | MCL1 |
| HALLMARK_TNFA_SIGNALING_VIA_NFKB | 9242 | MSC |
| HALLMARK_TNFA_SIGNALING_VIA_NFKB | 10725 | NFAT5 |
| HALLMARK_TNFA_SIGNALING_VIA_NFKB | 4780 | NFE2L2 |
| HALLMARK_TNFA_SIGNALING_VIA_NFKB | 4791 | NFKB2 |
| HALLMARK_TNFA_SIGNALING_VIA_NFKB | 4794 | NFKBIE |
| HALLMARK_TNFA_SIGNALING_VIA_NFKB | 4814 | NINJ1 |
| HALLMARK_TNFA_SIGNALING_VIA_NFKB | 3164 | NR4A1 |
| HALLMARK_TNFA_SIGNALING_VIA_NFKB | 4929 | NR4A2 |
| HALLMARK_TNFA_SIGNALING_VIA_NFKB | 8013 | NR4A3 |
| HALLMARK_TNFA_SIGNALING_VIA_NFKB | 24145 | PANX1 |
| HALLMARK_TNFA_SIGNALING_VIA_NFKB | 10611 | PDLIM5 |
| HALLMARK_TNFA_SIGNALING_VIA_NFKB | 5187 | PER1 |
| HALLMARK_TNFA_SIGNALING_VIA_NFKB | 22822 | PHLDA1 |
| HALLMARK_TNFA_SIGNALING_VIA_NFKB | 7262 | PHLDA2 |
| HALLMARK_TNFA_SIGNALING_VIA_NFKB | 5341 | PLEK |
| HALLMARK_TNFA_SIGNALING_VIA_NFKB | 10769 | PLK2 |
| HALLMARK_TNFA_SIGNALING_VIA_NFKB | 56937 | PMEPA1 |
| HALLMARK_TNFA_SIGNALING_VIA_NFKB | 8613 | PLPP3 |
| HALLMARK_TNFA_SIGNALING_VIA_NFKB | 5806 | PTX3 |
| HALLMARK_TNFA_SIGNALING_VIA_NFKB | 1827 | RCAN1 |
| HALLMARK_TNFA_SIGNALING_VIA_NFKB | 5966 | REL |
| HALLMARK_TNFA_SIGNALING_VIA_NFKB | 5971 | RELB |
| HALLMARK_TNFA_SIGNALING_VIA_NFKB | 388 | RHOB |
| HALLMARK_TNFA_SIGNALING_VIA_NFKB | 127544 | RNF19B |
| HALLMARK_TNFA_SIGNALING_VIA_NFKB | 6303 | SAT1 |
| HALLMARK_TNFA_SIGNALING_VIA_NFKB | 5055 | SERPINB2 |
| HALLMARK_TNFA_SIGNALING_VIA_NFKB | 5271 | SERPINB8 |
| HALLMARK_TNFA_SIGNALING_VIA_NFKB | 6446 | SGK1 |
| HALLMARK_TNFA_SIGNALING_VIA_NFKB | 150094 | SIK1 |
| HALLMARK_TNFA_SIGNALING_VIA_NFKB | 9120 | SLC16A6 |
| HALLMARK_TNFA_SIGNALING_VIA_NFKB | 11182 | SLC2A6 |
| HALLMARK_TNFA_SIGNALING_VIA_NFKB | 4088 | SMAD3 |
| HALLMARK_TNFA_SIGNALING_VIA_NFKB | 8303 | SNN |
| HALLMARK_TNFA_SIGNALING_VIA_NFKB | 9021 | SOCS3 |
| HALLMARK_TNFA_SIGNALING_VIA_NFKB | 6648 | SOD2 |
| HALLMARK_TNFA_SIGNALING_VIA_NFKB | 80176 | SPSB1 |
| HALLMARK_TNFA_SIGNALING_VIA_NFKB | 6776 | STAT5A |
| HALLMARK_TNFA_SIGNALING_VIA_NFKB | 10010 | TANK |
| HALLMARK_TNFA_SIGNALING_VIA_NFKB | 6890 | TAP1 |
| HALLMARK_TNFA_SIGNALING_VIA_NFKB | 7050 | TGIF1 |
| HALLMARK_TNFA_SIGNALING_VIA_NFKB | 3371 | TNC |
| HALLMARK_TNFA_SIGNALING_VIA_NFKB | 7124 | TNF |
| HALLMARK_TNFA_SIGNALING_VIA_NFKB | 7127 | TNFAIP2 |
| HALLMARK_TNFA_SIGNALING_VIA_NFKB | 25816 | TNFAIP8 |
| HALLMARK_TNFA_SIGNALING_VIA_NFKB | 10318 | TNIP1 |
| HALLMARK_TNFA_SIGNALING_VIA_NFKB | 79155 | TNIP2 |
| HALLMARK_TNFA_SIGNALING_VIA_NFKB | 9322 | TRIP10 |
| HALLMARK_TNFA_SIGNALING_VIA_NFKB | 8848 | TSC22D1 |
| HALLMARK_TNFA_SIGNALING_VIA_NFKB | 7280 | TUBB2A |
| HALLMARK_TNFA_SIGNALING_VIA_NFKB | 65986 | ZBTB10 |
| HALLMARK_TNFA_SIGNALING_VIA_NFKB | 80149 | ZC3H12A |
| HALLMARK_TNFA_SIGNALING_VIA_NFKB | 467 | ATF3 |
| HALLMARK_{—}TNFA_{—}SIGNALING_{—}VIA_{—}NFKB; | 8553 | BHLHE40 |
| HALLMARK_{—}HYPOXIA | | |
| HALLMARK_{—}TNFA_{—}SIGNALING_{—}VIA_{—}NFKB; | 694 | BTG1 |
| HALLMARK_{—}HYPOXIA | | |
| HALLMARK_{—}TNFA_{—}SIGNALING_{—}VIA_{—}NFKB; | 57007 | ACKR3 |
| HALLMARK_{—}HYPOXIA | | |
| HALLMARK_{—}TNFA_{—}SIGNALING_{—}VIA_{—}NFKB; | 3491 | CCN1 |
| HALLMARK_{—}HYPOXIA | | |
| HALLMARK_{—}TNFA_{—}SIGNALING_{—}VIA_{—}NFKB; | 1843 | DUSP1 |
| HALLMARK_{—}HYPOXIA | | |
| HALLMARK_{—}TNFA_{—}SIGNALING_{—}VIA_{—}NFKB; | 1942 | EFNA1 |
| HALLMARK_{—}HYPOXIA | | |
| HALLMARK_{—}TNFA_{—}SIGNALING_{—}VIA_{—}NFKB; | 2353 | FOS |
| HALLMARK_{—}HYPOXIA | | |
| HALLMARK_{—}TNFA_{—}SIGNALING_{—}VIA_{—}NFKB; | 2355 | FOSL2 |
| HALLMARK_{—}HYPOXIA | | |
| HALLMARK_{—}TNFA_{—}SIGNALING_{—}VIA_{—}NFKB; | 8870 | IER3 |
| HALLMARK_{—}HYPOXIA | | |
| HALLMARK_{—}TNFA_{—}SIGNALING_{—}VIA_{—}NFKB; | 8660 | IRS2 |
| HALLMARK_{—}HYPOXIA | | |
| HALLMARK_{—}TNFA_{—}SIGNALING_{—}VIA_{—}NFKB; | 3725 | JUN |
| HALLMARK_{—}HYPOXIA | | |
| HALLMARK_{—}TNFA_{—}SIGNALING_{—}VIA_{—}NFKB; | 23764 | MAFF |
| HALLMARK_{—}HYPOXIA | | |
| HALLMARK_{—}TNFA_{—}SIGNALING_{—}VIA_{—}NFKB; | 4783 | NFIL3 |
| HALLMARK_{—}HYPOXIA | | |
| HALLMARK_{—}TNFA_{—}SIGNALING_{—}VIA_{—}NFKB; | 5209 | PFKFB3 |
| HALLMARK_{—}HYPOXIA | | |
| HALLMARK_{—}TNFA_{—}SIGNALING_{—}VIA_{—}NFKB; | 10957 | PNRC1 |
| HALLMARK_{—}HYPOXIA | | |
| HALLMARK_{—}TNFA_{—}SIGNALING_{—}VIA_{—}NFKB; | 6385 | SDC4 |
| HALLMARK_{—}HYPOXIA | | |
| HALLMARK_{—}TNFA_{—}SIGNALING_{—}VIA_{—}NFKB; | 6515 | SLC2A3 |
| HALLMARK_{—}HYPOXIA | | |
| HALLMARK_{—}TNFA_{—}SIGNALING_{—}VIA_{—}NFKB; | 25976 | TIPARP |
| HALLMARK_{—}HYPOXIA | | |
| HALLMARK_{—}TNFA_{—}SIGNALING_{—}VIA_{—}NFKB; | 7422 | VEGFA |
| HALLMARK_{—}HYPOXIA | | |
| HALLMARK_{—}TNFA_{—}SIGNALING_{—}VIA_{—}NFKB; | 7538 | ZFP36 |
| HALLMARK_{—}HYPOXIA | | |
| HALLMARK_{—}TNFA_{—}SIGNALING_{—}VIA_{—}NFKB; | 2152 | F3 |
| HALLMARK_{—}HYPOXIA | | |
| HALLMARK_{—}TNFA_{—}SIGNALING_{—}VIA_{—}NFKB; | 3569 | IL6 |
| HALLMARK_{—}HYPOXIA | | |
| HALLMARK_{—}INFLAMMATORY_{—}RESPONSE | | |
| HALLMARK_{—}TNFA_{—}SIGNALING_{—}VIA_{—}NFKB; | 1316 | KLF6 |
| HALLMARK_{—}HYPOXIA | | |
| HALLMARK_{—}INFLAMMATORY_{—}RESPONSE | | |
| HALLMARK_{—}TNFA_{—}SIGNALING_{—}VIA_{—}NFKB; | 5054 | SERPINE1 |
| HALLMARK_{—}HYPOXIA | | |
| HALLMARK_{—}INFLAMMATORY_{—}RESPONSE | | |
| HALLMARK_{—}TNFA_{—}SIGNALING_{—}VIA_{—}NFKB; | 5329 | PLAUR |
| HALLMARK_{—}HYPOXIA | | |
| HALLMARK_{—}INFLAMMATORY_{—}RESPONSE | | |
| HALLMARK_{—}TNFA_{—}SIGNALING_{—}VIA_{—}NFKB; | 23645 | PPP1R15A |
| HALLMARK_{—}HYPOXIA | | |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 7128 | TNFAIP3 |
| HALLMARK_{—}HYPOXIA; | | |
| HALLMARK_{—}KRAS_{—}SIGNALING_{—}UP | | |
| HALLMARK_{—}TNFA_{—}SIGNALING_{—}VIA_{—}NFKB; | 1026 | CDKN1A |
| HALLMARK_{—}HYPOXIA; | | |
| HALLMARK_{—}KRAS_{—}SIGNALING_{—}UP | | |
| HALLMARK_{—}TNFA_{—}SIGNALING_{—}VIA_{—}NFKB; | 19 | ABCA1 |
| HALLMARK_{—}HYPOXIA; | | |
| HALLMARK_{—}PI3K_{—}AKT_{—}MTOR_{—}SIGNALING; | | |
| HALLMARK_{—}INFLAMMATORY_{—}RESPONSE | | |
| HALLMARK_{—}TNFA_{—}SIGNALING_{—}VIA_{—}NFKB; | 490 | ATP2B1 |
| HALLMARK_{—}INFLAMMATORY_{—}RESPONSE | | |
| HALLMARK_{—}TNFA_{—}SIGNALING_{—}VIA_{—}NFKB; | 7832 | BTG2 |
| HALLMARK_{—}INFLAMMATORY_{—}RESPONSE | | |
| HALLMARK_{—}TNFA_{—}SIGNALING_{—}VIA_{—}NFKB; | 6347 | CCL2 |
| HALLMARK_{—}INFLAMMATORY_{—}RESPONSE | | |
| HALLMARK_{—}TNFA_{—}SIGNALING_{—}VIA_{—}NFKB; | 6352 | CCL5 |
| HALLMARK_{—}INFLAMMATORY_{—}RESPONSE | | |
| HALLMARK_{—}TNFA_{—}SIGNALING_{—}VIA_{—}NFKB; | 9034 | CCRL2 |
| HALLMARK_{—}INFLAMMATORY_{—}RESPONSE | | |
| HALLMARK_{—}TNFA_{—}SIGNALING_{—}VIA_{—}NFKB; | 969 | CD69 |
| HALLMARK_{—}INFLAMMATORY_{—}RESPONSE | | |
| HALLMARK_{—}TNFA_{—}SIGNALING_{—}VIA_{—}NFKB; | 1435 | CSF1 |
| HALLMARK_{—}INFLAMMATORY_{—}RESPONSE | | |
| HALLMARK_{—}TNFA_{—}SIGNALING_{—}VIA_{—}NFKB; | 6373 | CXCL11 |
| HALLMARK_{—}INFLAMMATORY_{—}RESPONSE | | |
| HALLMARK_{—}TNFA_{—}SIGNALING_{—}VIA_{—}NFKB; | 6372 | CXCL6 |
| HALLMARK_{—}INFLAMMATORY_{—}RESPONSE | | |
| HALLMARK_{—}TNFA_{—}SIGNALING_{—}VIA_{—}NFKB; | 1906 | EDN1 |
| HALLMARK_{—}INFLAMMATORY_{—}RESPONSE | | |
| HALLMARK_{—}TNFA_{—}SIGNALING_{—}VIA_{—}NFKB; | 2643 | GCH1 |
| HALLMARK_{—}INFLAMMATORY_{—}RESPONSE | | |
| HALLMARK_{—}TNFA_{—}SIGNALING_{—}VIA_{—}NFKB; | 1880 | GPR183 |
| HALLMARK_{—}INFLAMMATORY_{—}RESPONSE | | |
| HALLMARK_{—}TNFA_{—}SIGNALING_{—}VIA_{—}NFKB; | 3383 | ICAM1 |
| HALLMARK_{—}INFLAMMATORY_{—}RESPONSE | | |
| HALLMARK_{—}TNFA_{—}SIGNALING_{—}VIA_{—}NFKB; | 23308 | ICOSLG |
| HALLMARK_{—}INFLAMMATORY_{—}RESPONSE | | |
| HALLMARK_{—}TNFA_{—}SIGNALING_{—}VIA_{—}NFKB; | 3460 | IFNGR2 |
| HALLMARK_{—}INFLAMMATORY_{—}RESPONSE | | |
| HALLMARK_{—}TNFA_{—}SIGNALING_{—}VIA_{—}NFKB; | 3593 | IL12B |
| HALLMARK_{—}INFLAMMATORY_{—}RESPONSE | | |
| HALLMARK_{—}TNFA_{—}SIGNALING_{—}VIA_{—}NFKB; | 3601 | IL15RA |
| HALLMARK_{—}INFLAMMATORY_{—}RESPONSE | | |
| HALLMARK_{—}TNFA_{—}SIGNALING_{—}VIA_{—}NFKB; | 3606 | IL18 |
| HALLMARK_{—}INFLAMMATORY_{—}RESPONSE | | |
| HALLMARK_{—}TNFA_{—}SIGNALING_{—}VIA_{—}NFKB; | 3552 | IL1A |
| HALLMARK_{—}INFLAMMATORY_{—}RESPONSE | | |
| HALLMARK_{—}TNFA_{—}SIGNALING_{—}VIA_{—}NFKB; | 3659 | IRF1 |
| HALLMARK_{—}INFLAMMATORY_{—}RESPONSE | | |
| HALLMARK_{—}TNFA_{—}SIGNALING_{—}VIA_{—}NFKB; | 3949 | LDLR |
| HALLMARK_{—}INFLAMMATORY_{—}RESPONSE | | |
| HALLMARK_{—}TNFA_{—}SIGNALING_{—}VIA_{—}NFKB; | 4084 | MXD1 |
| HALLMARK_{—}INFLAMMATORY_{—}RESPONSE | | |
| HALLMARK_{—}TNFA_{—}SIGNALING_{—}VIA_{—}NFKB; | 4609 | MYC |
| HALLMARK_{—}INFLAMMATORY_{—}RESPONSE | | |
| HALLMARK_{—}TNFA_{—}SIGNALING_{—}VIA_{—}NFKB; | 10135 | NAMPT |
| HALLMARK_{—}INFLAMMATORY_{—}RESPONSE | | |
| HALLMARK_{—}TNFA_{—}SIGNALING_{—}VIA_{—}NFKB; | 4790 | NFKB1 |
| HALLMARK_{—}INFLAMMATORY_{—}RESPONSE | | |
| HALLMARK_{—}TNFA_{—}SIGNALING_{—}VIA_{—}NFKB; | 4792 | NFKBIA |
| HALLMARK_{—}INFLAMMATORY_{—}RESPONSE | | |
| HALLMARK_{—}TNFA_{—}SIGNALING_{—}VIA_{—}NFKB; | 4973 | OLR1 |
| HALLMARK_{—}INFLAMMATORY_{—}RESPONSE | | |
| HALLMARK_{—}TNFA_{—}SIGNALING_{—}VIA_{—}NFKB; | 5142 | PDE4B |
| HALLMARK_{—}INFLAMMATORY_{—}RESPONSE | | |
| HALLMARK_{—}TNFA_{—}SIGNALING_{—}VIA_{—}NFKB; | 5734 | PTGER4 |
| HALLMARK_{—}INFLAMMATORY_{—}RESPONSE | | |
| HALLMARK_{—}TNFA_{—}SIGNALING_{—}VIA_{—}NFKB; | 5791 | PTPRE |
| HALLMARK_{—}INFLAMMATORY_{—}RESPONSE | | |
| HALLMARK_{—}TNFA_{—}SIGNALING_{—}VIA_{—}NFKB; | 5970 | RELA |
| HALLMARK_{—}INFLAMMATORY_{—}RESPONSE | | |
| HALLMARK_{—}TNFA_{—}SIGNALING_{—}VIA_{—}NFKB; | 8767 | RIPK2 |
| HALLMARK_{—}INFLAMMATORY_{—}RESPONSE | | |
| HALLMARK_{—}TNFA_{—}SIGNALING_{—}VIA_{—}NFKB; | 8877 | SPHK1 |
| HALLMARK_{—}INFLAMMATORY_{—}RESPONSE | | |
| HALLMARK_{—}TNFA_{—}SIGNALING_{—}VIA_{—}NFKB; | 7097 | TLR2 |
| HALLMARK_{—}INFLAMMATORY_{—}RESPONSE | | |
| HALLMARK_{—}TNFA_{—}SIGNALING_{—}VIA_{—}NFKB; | 7130 | TNFAIP6 |
| HALLMARK_{—}INFLAMMATORY_{—}RESPONSE | | |
| HALLMARK_{—}TNFA_{—}SIGNALING_{—}VIA_{—}NFKB; | 3604 | TNFRSF9 |
| HALLMARK_{—}INFLAMMATORY_{—}RESPONSE | | |
| HALLMARK_{—}TNFA_{—}SIGNALING_{—}VIA_{—}NFKB; | 8744 | TNFSF9 |
| HALLMARK_{—}INFLAMMATORY_{—}RESPONSE | | |
| HALLMARK_{—}TNFA_{—}SIGNALING_{—}VIA_{—}NFKB; | 6364 | CCL20 |
| HALLMARK_{—}INFLAMMATORY_{—}RESPONSE | | |
| HALLMARK_{—}TNFA_{—}SIGNALING_{—}VIA_{—}NFKB; | 3627 | CXCL10 |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 1839 | HBEGF |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 3553 | IL1B |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 3575 | IL7R |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 3624 | INHBA |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 3976 | LIF |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 330 | BIRC3 |
| HALLMARK_{—}INFLAMMATORY_{—}RESPONSE | | |
| HALLMARK_{—}KRAS_{—}SIGNALING_{—}UP | | |
| HALLMARK_{—}TNFA_{—}SIGNALING_{—}VIA_{—}NFKB; | 650 | BMP2 |
| HALLMARK_{—}KRAS_{—}SIGNALING_{—}UP | | |
| HALLMARK_{—}TNFA_{—}SIGNALING_{—}VIA_{—}NFKB; | 1437 | CSF2 |
| HALLMARK_{—}KRAS_{—}SIGNALING_{—}UP | | |
| HALLMARK_{—}TNFA_{—}SIGNALING_{—}VIA_{—}NFKB; | 2150 | F2RL1 |
| HALLMARK_{—}KRAS_{—}SIGNALING_{—}UP | | |
| HALLMARK_{—}TNFA_{—}SIGNALING_{—}VIA_{—}NFKB; | 50486 | G0S2 |
| HALLMARK_{—}KRAS_{—}SIGNALING_{—}UP | | |
| HALLMARK_{—}TNFA_{—}SIGNALING_{—}VIA_{—}NFKB; | 9945 | GFPT2 |
| HALLMARK_{—}KRAS_{—}SIGNALING_{—}UP | | |
| HALLMARK_{—}TNFA_{—}SIGNALING_{—}VIA_{—}NFKB; | 3398 | ID2 |
| HALLMARK_{—}KRAS_{—}SIGNALING_{—}UP | | |
| HALLMARK_{—}TNFA_{—}SIGNALING_{—}VIA_{—}NFKB; | 9314 | KLF4 |
| HALLMARK_{—}KRAS_{—}SIGNALING_{—}UP | | |
| HALLMARK_{—}TNFA_{—}SIGNALING_{—}VIA_{—}NFKB; | 5328 | PLAU |
| HALLMARK_{—}KRAS_{—}SIGNALING_{—}UP | | |
| HALLMARK_{—}TNFA_{—}SIGNALING_{—}VIA_{—}NFKB; | 5743 | PTGS2 |
| HALLMARK_{—}KRAS_{—}SIGNALING_{—}UP | | |
| HALLMARK_{—}TNFA_{—}SIGNALING_{—}VIA_{—}NFKB; | 7185 | TRAF1 |
| HALLMARK_{—}KRAS_{—}SIGNALING_{—}UP | | |
| HALLMARK_{—}TNFA_{—}SIGNALING_{—}VIA_{—}NFKB; | 10221 | TRIB1 |
| HALLMARK_{—}KRAS_{—}SIGNALING_{—}UP | | |
| HALLMARK_{—}TNFA_{—}SIGNALING_{—}VIA_{—}NFKB; | 79693 | YRDC |
| HALLMARK_{—}KRAS_{—}SIGNALING_{—}UP | | |
| HALLMARK_{—}TNFA_{—}SIGNALING_{—}VIA_{—}NFKB; | 5606 | MAP2K3 |
| HALLMARK_{—}KRAS_{—}SIGNALING_{—}UP | | |
| HALLMARK_{—}TNFA_{—}SIGNALING_{—}VIA_{—}NFKB; | 8878 | SQSTM1 |
| HALLMARK_{—}PI3K_{—}AKT_{—}MTOR_{—}SIGNALING | | |
| HALLMARK_{—}TNFA_{—}SIGNALING_{—}VIA_{—}NFKB; | 6833 | ABCC8 |
| HALLMARK_{—}PI3K_{—}AKT_{—}MTOR_{—}SIGNALING | | |
| HALLMARK_PANCREAS_BETA_CELLS | 10000 | AKT3 |
| HALLMARK_PANCREAS_BETA_CELLS | 1113 | CHGA |
| HALLMARK_PANCREAS_BETA_CELLS | 1641 | DCX |
| HALLMARK_PANCREAS_BETA_CELLS | 1803 | DPP4 |
| HALLMARK_PANCREAS_BETA_CELLS | 26610 | ELP4 |
| HALLMARK_PANCREAS_BETA_CELLS | 3170 | FOXA2 |
| HALLMARK_PANCREAS_BETA_CELLS | 2308 | FOXO1 |
| HALLMARK_PANCREAS_BETA_CELLS | 57818 | G6PC2 |
| HALLMARK_PANCREAS_BETA_CELLS | 2641 | GCG |
| HALLMARK_PANCREAS_BETA_CELLS | 6927 | HNF1A |
| HALLMARK_PANCREAS_BETA_CELLS | 3375 | IAPP |
| HALLMARK_PANCREAS_BETA_CELLS | 3630 | INS |
| HALLMARK_PANCREAS_BETA_CELLS | 3642 | INSM1 |
| HALLMARK_PANCREAS_BETA_CELLS | 3670 | ISL1 |
| HALLMARK_PANCREAS_BETA_CELLS | 4005 | LMO2 |
| HALLMARK_PANCREAS_BETA_CELLS | 4760 | NEUROD1 |
| HALLMARK_PANCREAS_BETA_CELLS | 50674 | NEUROG3 |
| HALLMARK_PANCREAS_BETA_CELLS | 4821 | NKX2-2 |
| HALLMARK_PANCREAS_BETA_CELLS | 4825 | NKX6-1 |
| HALLMARK_PANCREAS_BETA_CELLS | 5063 | PAK3 |
| HALLMARK_PANCREAS_BETA_CELLS | 5078 | PAX4 |
| HALLMARK_PANCREAS_BETA_CELLS | 5080 | PAX6 |
| HALLMARK_PANCREAS_BETA_CELLS | 5122 | PCSK1 |
| HALLMARK_PANCREAS_BETA_CELLS | 5126 | PCSK2 |
| HALLMARK_PANCREAS_BETA_CELLS | 3651 | PDX1 |
| HALLMARK_PANCREAS_BETA_CELLS | 10590 | SCGN |
| HALLMARK_PANCREAS_BETA_CELLS | 23478 | SEC11A |
| HALLMARK_PANCREAS_BETA_CELLS | 6514 | SLC2A2 |
| HALLMARK_PANCREAS_BETA_CELLS | 28972 | SPCS1 |
| HALLMARK_PANCREAS_BETA_CELLS | 6727 | SRP14 |
| HALLMARK_PANCREAS_BETA_CELLS | 6726 | SRP9 |
| HALLMARK_PANCREAS_BETA_CELLS | 58477 | SRPRB |
| HALLMARK_PANCREAS_BETA_CELLS | 6750 | SST |
| HALLMARK_PANCREAS_BETA_CELLS | 6812 | STXBP1 |
| HALLMARK_PANCREAS_BETA_CELLS | 57586 | SYT13 |
| HALLMARK_PANCREAS_BETA_CELLS | 7421 | VDR |
| HALLMARK_PANCREAS_BETA_CELLS | 10006 | ABI1 |
| HALLMARK_INFLAMMATORY_RESPONSE | 91 | ACVR1B |
| HALLMARK_INFLAMMATORY_RESPONSE | 92 | ACVR2A |
| HALLMARK_INFLAMMATORY_RESPONSE | 11047 | ADRM1 |
| HALLMARK_INFLAMMATORY_RESPONSE | 196 | AHR |
| HALLMARK_INFLAMMATORY_RESPONSE | 187 | APLNR |
| HALLMARK_INFLAMMATORY_RESPONSE | 366 | AQP9 |
| HALLMARK_INFLAMMATORY_RESPONSE | 488 | ATP2A2 |
| HALLMARK_INFLAMMATORY_RESPONSE | 27032 | ATP2C1 |
| HALLMARK_INFLAMMATORY_RESPONSE | 558 | AXL |
| HALLMARK_INFLAMMATORY_RESPONSE | 623 | BDKRB1 |
| HALLMARK_INFLAMMATORY_RESPONSE | 7439 | BEST1 |
| HALLMARK_INFLAMMATORY_RESPONSE | 684 | BST2 |
| HALLMARK_INFLAMMATORY_RESPONSE | 728 | C5AR1 |
| HALLMARK_INFLAMMATORY_RESPONSE | 10203 | CALCRL |
| HALLMARK_INFLAMMATORY_RESPONSE | 6361 | CCL17 |
| HALLMARK_INFLAMMATORY_RESPONSE | 6367 | CCL22 |
| HALLMARK_INFLAMMATORY_RESPONSE | 6369 | CCL24 |
| HALLMARK_INFLAMMATORY_RESPONSE | 6354 | CCL7 |
| HALLMARK_INFLAMMATORY_RESPONSE | 1236 | CCR7 |
| HALLMARK_INFLAMMATORY_RESPONSE | 929 | CD14 |
| HALLMARK_INFLAMMATORY_RESPONSE | 958 | CD40 |
| HALLMARK_INFLAMMATORY_RESPONSE | 962 | CD48 |
| HALLMARK_INFLAMMATORY_RESPONSE | 1604 | CD55 |
| HALLMARK_INFLAMMATORY_RESPONSE | 970 | CD70 |
| HALLMARK_INFLAMMATORY_RESPONSE | 3732 | CD82 |
| HALLMARK_INFLAMMATORY_RESPONSE | 23601 | CLEC5A |
| HALLMARK_INFLAMMATORY_RESPONSE | 1440 | CSF3 |
| HALLMARK_INFLAMMATORY_RESPONSE | 1441 | CSF3R |
| HALLMARK_INFLAMMATORY_RESPONSE | 6376 | CX3CL1 |
| HALLMARK_INFLAMMATORY_RESPONSE | 4283 | CXCL9 |
| HALLMARK_INFLAMMATORY_RESPONSE | 10663 | CXCR6 |
| HALLMARK_INFLAMMATORY_RESPONSE | 1536 | CYBB |
| HALLMARK_INFLAMMATORY_RESPONSE | 10148 | EBI3 |
| HALLMARK_INFLAMMATORY_RESPONSE | 5610 | EIF2AK2 |
| HALLMARK_INFLAMMATORY_RESPONSE | 2014 | EMP3 |
| HALLMARK_INFLAMMATORY_RESPONSE | 2015 | ADGRE1 |
| HALLMARK_INFLAMMATORY_RESPONSE | 2867 | FFAR2 |
| HALLMARK_INFLAMMATORY_RESPONSE | 2357 | FPR1 |
| HALLMARK_INFLAMMATORY_RESPONSE | 7855 | FZD5 |
| HALLMARK_INFLAMMATORY_RESPONSE | 2550 | GABBR1 |
| HALLMARK_INFLAMMATORY_RESPONSE | 2769 | GNA15 |
| HALLMARK_INFLAMMATORY_RESPONSE | 2773 | GNAI3 |
| HALLMARK_INFLAMMATORY_RESPONSE | 2811 | GP1BA |
| HALLMARK_INFLAMMATORY_RESPONSE | 29933 | GPR132 |
| HALLMARK_INFLAMMATORY_RESPONSE | 3037 | HAS2 |
| HALLMARK_INFLAMMATORY_RESPONSE | 3091 | HIF1A |
| HALLMARK_INFLAMMATORY_RESPONSE | 3249 | HPN |
| HALLMARK_INFLAMMATORY_RESPONSE | 3269 | HRH1 |
| HALLMARK_INFLAMMATORY_RESPONSE | 3386 | ICAM4 |
| HALLMARK_INFLAMMATORY_RESPONSE | 8519 | IFITM1 |
| HALLMARK_INFLAMMATORY_RESPONSE | 3454 | IFNAR1 |
| HALLMARK_INFLAMMATORY_RESPONSE | 3586 | IL10 |
| HALLMARK_INFLAMMATORY_RESPONSE | 3600 | IL15 |
| HALLMARK_INFLAMMATORY_RESPONSE | 8809 | IL18R1 |
| HALLMARK_INFLAMMATORY_RESPONSE | 8807 | IL18RAP |
| HALLMARK_INFLAMMATORY_RESPONSE | 3554 | IL1R1 |
| HALLMARK_INFLAMMATORY_RESPONSE | 3560 | IL2RB |
| HALLMARK_INFLAMMATORY_RESPONSE | 3566 | IL4R |
| HALLMARK_INFLAMMATORY_RESPONSE | 3576 | CXCL8 |
| HALLMARK_INFLAMMATORY_RESPONSE | 3656 | IRAK2 |
| HALLMARK_INFLAMMATORY_RESPONSE | 3665 | IRF7 |
| HALLMARK_INFLAMMATORY_RESPONSE | 3678 | ITGA5 |
| HALLMARK_INFLAMMATORY_RESPONSE | 3690 | ITGB3 |
| HALLMARK_INFLAMMATORY_RESPONSE | 3696 | ITGB8 |
| HALLMARK_INFLAMMATORY_RESPONSE | 3738 | KCNA3 |
| HALLMARK_INFLAMMATORY_RESPONSE | 3759 | KCNJ2 |
| HALLMARK_INFLAMMATORY_RESPONSE | 10242 | KCNMB2 |
| HALLMARK_INFLAMMATORY_RESPONSE | 23095 | KIF1B |
| HALLMARK_INFLAMMATORY_RESPONSE | 27074 | LAMP3 |
| HALLMARK_INFLAMMATORY_RESPONSE | 3937 | LCP2 |
| HALLMARK_INFLAMMATORY_RESPONSE | 1902 | LPAR1 |
| HALLMARK_INFLAMMATORY_RESPONSE | 4049 | LTA |
| HALLMARK_INFLAMMATORY_RESPONSE | 4061 | LY6E |
| HALLMARK_INFLAMMATORY_RESPONSE | 4067 | LYN |
| HALLMARK_INFLAMMATORY_RESPONSE | 8685 | MARCO |
| HALLMARK_INFLAMMATORY_RESPONSE | 4210 | MEFV |
| HALLMARK_INFLAMMATORY_RESPONSE | 4224 | MEP1A |
| HALLMARK_INFLAMMATORY_RESPONSE | 4233 | MET |
| HALLMARK_INFLAMMATORY_RESPONSE | 4323 | MMP14 |
| HALLMARK_INFLAMMATORY_RESPONSE | 4481 | MSR1 |
| HALLMARK_INFLAMMATORY_RESPONSE | 4693 | NDP |
| HALLMARK_INFLAMMATORY_RESPONSE | 114548 | NLRP3 |
| HALLMARK_INFLAMMATORY_RESPONSE | 9111 | NMI |
| HALLMARK_INFLAMMATORY_RESPONSE | 10316 | NMUR1 |
| HALLMARK_INFLAMMATORY_RESPONSE | 64127 | NOD2 |
| HALLMARK_INFLAMMATORY_RESPONSE | 10886 | NPFFR2 |
| HALLMARK_INFLAMMATORY_RESPONSE | 4986 | OPRK1 |
| HALLMARK_INFLAMMATORY_RESPONSE | 5008 | OSM |
| HALLMARK_INFLAMMATORY_RESPONSE | 9180 | OSMR |
| HALLMARK_INFLAMMATORY_RESPONSE | 5025 | P2RX4 |
| HALLMARK_INFLAMMATORY_RESPONSE | 5027 | P2RX7 |
| HALLMARK_INFLAMMATORY_RESPONSE | 5029 | P2RY2 |
| HALLMARK_INFLAMMATORY_RESPONSE | 5099 | PCDH7 |
| HALLMARK_INFLAMMATORY_RESPONSE | 10630 | PDPN |
| HALLMARK_INFLAMMATORY_RESPONSE | 23533 | PIK3R5 |
| HALLMARK_INFLAMMATORY_RESPONSE | 60675 | PROK2 |
| HALLMARK_INFLAMMATORY_RESPONSE | 5663 | PSEN1 |
| HALLMARK_INFLAMMATORY_RESPONSE | 5724 | PTAFR |
| HALLMARK_INFLAMMATORY_RESPONSE | 5732 | PTGER2 |
| HALLMARK_INFLAMMATORY_RESPONSE | 5739 | PTGIR |
| HALLMARK_INFLAMMATORY_RESPONSE | 5817 | PVR |
| HALLMARK_INFLAMMATORY_RESPONSE | 10125 | RASGRP1 |
| HALLMARK_INFLAMMATORY_RESPONSE | 5996 | RGS1 |
| HALLMARK_INFLAMMATORY_RESPONSE | 391 | RHOG |
| HALLMARK_INFLAMMATORY_RESPONSE | 255488 | RNF144B |
| HALLMARK_INFLAMMATORY_RESPONSE | 6098 | ROS1 |
| HALLMARK_INFLAMMATORY_RESPONSE | 64108 | RTP4 |
| HALLMARK_INFLAMMATORY_RESPONSE | 8578 | SCARF1 |
| HALLMARK_INFLAMMATORY_RESPONSE | 6401 | SELE |
| HALLMARK_INFLAMMATORY_RESPONSE | 6402 | SELL |
| HALLMARK_INFLAMMATORY_RESPONSE | 55829 | SELENOS |
| HALLMARK_INFLAMMATORY_RESPONSE | 10507 | SEMA4D |
| HALLMARK_INFLAMMATORY_RESPONSE | 166929 | SGMS2 |
| HALLMARK_INFLAMMATORY_RESPONSE | 6504 | SLAMF1 |
| HALLMARK_INFLAMMATORY_RESPONSE | 4891 | SLC11A2 |
| HALLMARK_INFLAMMATORY_RESPONSE | 6506 | SLC1A2 |
| HALLMARK_INFLAMMATORY_RESPONSE | 9153 | SLC28A2 |
| HALLMARK_INFLAMMATORY_RESPONSE | 1317 | SLC31A1 |
| HALLMARK_INFLAMMATORY_RESPONSE | 1318 | SLC31A2 |
| HALLMARK_INFLAMMATORY_RESPONSE | 8671 | SLC4A4 |
| HALLMARK_INFLAMMATORY_RESPONSE | 6541 | SLC7A1 |
| HALLMARK_INFLAMMATORY_RESPONSE | 6542 | SLC7A2 |
| HALLMARK_INFLAMMATORY_RESPONSE | 6717 | SRI |
| HALLMARK_INFLAMMATORY_RESPONSE | 23166 | STAB1 |
| HALLMARK_INFLAMMATORY_RESPONSE | 6869 | TACR1 |
| HALLMARK_INFLAMMATORY_RESPONSE | 6870 | TACR3 |
| HALLMARK_INFLAMMATORY_RESPONSE | 6892 | TAPBP |
| HALLMARK_INFLAMMATORY_RESPONSE | 7076 | TIMP1 |
| HALLMARK_INFLAMMATORY_RESPONSE | 7096 | TLR1 |
| HALLMARK_INFLAMMATORY_RESPONSE | 7098 | TLR3 |
| HALLMARK_INFLAMMATORY_RESPONSE | 8743 | TNFSF10 |
| HALLMARK_INFLAMMATORY_RESPONSE | 9966 | TNFSF15 |
| HALLMARK_INFLAMMATORY_RESPONSE | 7432 | VIP |
| HALLMARK_INFLAMMATORY_RESPONSE | 719 | C3AR1 |
| HALLMARK_{—}INFLAMMATORY_{—}RESPONSE; | 1240 | CMKLR1 |
| HALLMARK_{—}KRAS_{—}SIGNALING_{—}UP | | |
| HALLMARK_{—}INFLAMMATORY_{—}RESPONSE; | 131566 | DCBLD2 |
| HALLMARK_{—}KRAS_{—}SIGNALING_{—}UP | | |
| HALLMARK_{—}INFLAMMATORY_{—}RESPONSE; | 2069 | EREG |
| HALLMARK_{—}KRAS_{—}SIGNALING_{—}UP | | |
| HALLMARK_{—}INFLAMMATORY_{—}RESPONSE; | 3587 | IL10RA |
| HALLMARK_{—}KRAS_{—}SIGNALING_{—}UP | | |
| HALLMARK_{—}INFLAMMATORY_{—}RESPONSE; | 6004 | RGS16 |
| HALLMARK_{—}KRAS_{—}SIGNALING_{—}UP | | |
| HALLMARK_{—}INFLAMMATORY_{—}RESPONSE; | 6324 | SCN1B |
| HALLMARK_{—}KRAS_{—}SIGNALING_{—}UP | | |
| HALLMARK_{—}INFLAMMATORY_{—}RESPONSE; | 7133 | TNFRSF1B |
| HALLMARK_{—}KRAS_{—}SIGNALING_{—}UP | | |
| HALLMARK_{—}INFLAMMATORY_{—}RESPONSE; | 9935 | MAFB |
| HALLMARK_{—}KRAS_{—}SIGNALING_{—}UP | | |

As can be seen in Table 1, the genes that may be analysed by methods of the invention may be grouped into gene sets according to the pathways to which the genes belong. The gene sets are available from the Molecular Signatures Database (MSigDB) hallmark gene set collection with each gene set contents available from https://www.gsea-msigdb.org/gsea/msigdb/genesets.jsp?collection=H the contents of which is expressly incorporated herein. The hallmark gene sets are also described in “Liberzon A, Birger C, Thorvaldsdóttir H, Ghandi M, Mesirov J P, Tamayo P. The Molecular Signatures Database (MSigDB) hallmark gene set collection. Cell Syst. 2015 Dec. 23; 1(6):417-425. doi: 10.1016/j.cels.2015.12.004. PMID: 26771021; PMCID: PMC4707969.” The contents of which is expressly incorporated herein.

Thus in some examples, the germline variant may be at least one variant of at least one gene in one or more of Hallmark gene set number:
M5891 (HALLMARK_HYPOXIA);
M5932 (HALLMARK_INFLAMMATORY_RESPONSE);
M5953 (HALLMARK_KRAS_SIGNALING_UP);
M5957 (HALLMARK_PANCREAS_BETA_CELLS);
M5923 (HALLMARK_PI3K_AKT_MTOR_SIGNALING); and/or
M5890 (HALLMARK_TNFA_SIGNALING_VIA_NFKB).

As used herein, the terms “variant”, “variant gene” and “gene variant” refer to any change in nucleotide sequence relative to the native or wild type sequences. These terms are used interchangeably with “mutant”, “mutant gene” and “gene mutation”. Examples include, but are not limited to, single nucleotide polymorphisms (SNPs), deletions, inversions, splice variants, frameshift variants, nonsense variants or haplotypes.

In some examples, the germline variant is an exome variant. For example, the germline variant is within a protein-coding sequence of a gene. In some examples, the germline variant is within a protein-coding transcript sequence. Determination of protein-coding transcript sequences may be done using a genome database such as GENCODE (e.g. Gencode v29). GENCODE gene annotations are accessible via the Ensembl and UCSC Genome Browsers, the Ensembl FTP site, Ensembl Biomart, Ensembl Perl and REST APIs as well as https://www.gencodegenes.org.

In some examples, the germline variant is a rare germline variant. For example, the germline variant may have a minor allele frequency (MAF) of less than 1%. The proportion of the second-most-common of two (or rarely, three) alleles at a genetic locus in a population, ranging from <1 to <50%. In some examples, the MAF of the germline variant is from 0.001% to 0.999%.

In some examples the germline variant is a deleterious variant. In some examples the germline variant is a deleterious variant or mutation. “Deleterious mutation” and “deleterious variant” refer to variants or mutations that compromise or alter the normal function of a gene product for example by decreasing or increasing activity of the gene product or alters expression of the gene product in the subject for example by decreasing or increasing expression of the gene product. In some examples, the germline variant may be a loss of function variant or mutation. The term “loss of function mutation” refers to a mutation that results in a gene product no longer being able to perform its normal function or its normal level of activity, in whole or in part. Loss of function mutations are also referred to as inactivating mutations and typically result in the gene product having less or no function, i.e., being partially or wholly inactivated (e.g., a non-functional protein has less than 50%, 40%, 30%, 20%, 10%, 9%, 8%, 7%, 6%, 5%, 4%, 3%, 2%, 1% or less activity than its native or wild-type counterpart).

In some examples, the germline variant may be a likely deleterious variant. In some examples, the germline variant is a predicted deleterious mutant. Determination of whether a variant is likely to be deleterious or is predicted to be deleterious may be done using any suitable variant annotation tool. For example using, SnpEff, Combined Annotation Dependent Depletion (CADD), ANNOVAR, AnnTools, NGS-SNP, Sequence Variant Analyzer (SVA), SeattleSeq Annotation Server, Variant (VARIANT), Variant Effect Predictor (VEP) or combinations thereof. In some examples, the germline variant is predicted to be likely to be deleterious as determined by Variant Effect Predictor (VEP) see “Variant Effect Predictor,” Genome Biology 17, p. 122, doi: 10.1186/s13059-016-0974-4 each of which is hereby incorporated by reference.

In some examples, the germline variant may be a predicted or likely loss of function variant. Determination of predicted or likely loss of function variants may be done using any suitable annotation and/or prediction tools such as loss-of-function transcript effect estimator (LOFTEE) (available via https://github.com/konradjk/loftee).

In some examples, the germline variant is a protein-truncating variant or mutation. For example a protein-truncating loss of function and/or deleterious or predicted loss of function and/or deleterious mutation. Protein-truncating variants are genetic variants that are predicted to or do shorten the coding sequence of a gene, through for example a stop-gain mutation. Protein-truncating variants are sometimes categorized under the umbrella term frameshift or truncating variants (FTVs), which includes both Protein-truncating variants and DNA variants caused by frameshift mutation.

In some examples, the germline variant is a nonsense variant, frameshift variant or splice site variant. Nonsense mutation or variant refers to a mutation in which a sense codon that corresponds to one of the twenty amino acids specified by the genetic code is changed to a chain-terminating codon (i.e. stop codon). Frameshift mutation or variant refers to a mutation caused by the addition or deletion of a base pair or base pairs in the DNA of a gene resulting in the translation of the genetic code in an unnatural reading frame from the position of the mutation to the end of the gene. Splice site variant or mutation refers to a genetic alteration in the DNA sequence of a gene that occurs at the boundary of an exon and an intron (splice site). This change can disrupt RNA splicing resulting in the loss of exons or the inclusion of introns and an altered protein-coding sequence.

In some examples, the variation or mutation occurs in the first 95% of the protein encoded by the variant gene. For example a protein-truncating loss of function and/or deleterious or predicted loss of function and/or deleterious mutation occurring in the first 95% of a protein encoded by the gene.

In some examples, the germline variant may be a missense variant. A missense mutation or variant lead to a change in a single base pair that causes the substitution of an amino acid for a different amino acid in the resulting protein, in particular, a non-conservative amino acid substitution.

In some examples, the germline variant may be a gain-of-function or activating mutation. “Gain of function mutations” or “activating mutations refer to any mutation in a gene where the gene product (e.g. a protein) encoded and produced by expression of that gene acquires an unrelated function not normally associated with the wild-type gene product (i.e. the wild type protein) and Cause or contribute to a disease or disorder. For example, such mutations change the function of the resulting protein or causes interactions with other proteins. For example, a gain-of-function mutation changes the gene product such that its effect gets stronger (enhanced activation) or even is superseded by a different, abnormal function.

In some examples, the germline variant has a Combined Annotation Dependent Depletion (CADD) score or CADD phred score of greater than 30. The CADD tool scores the predicted deleteriousness of single nucleotide variants and insertion/deletions variants in the human genome by integrating multiple annotations including conservation and functional information into one metric. CADD provides a score that ranks genetic variants, including single nucleotide variants (SNVs) and short inserts and deletions (InDels), throughout the human genome reference assembly. CADD scores are based on diverse genomic features derived from surrounding sequence context, gene model annotations, evolutionary constraint, epigenetic measurements and functional predictions. For any given variant, all of these annotations are integrated into a single CADD score via a machine learning model. For improved interpretability, these are transformed into a PHRED-like (i.e. log 10-derived) rank score based on the genome-wide distribution of scores for all ˜9 billion potential SNVs, the set of all three non-reference alleles at each position of the reference assembly.

In some examples the germline variant is one or more variants of any one or more of the genes listed in table 2.

TABLE 2
Genes of M5923 (HALLMARK_PI3K_AKT_MTOR_SIGNALING).

| Original | NCBI GENE | Gene | |
|---|---|---|---|
| Member | ID | Symbol | Gene Description |
| ACACA | 31 | ACACA | acetyl-CoA carboxylase alpha [Source: HGN . . . |
| ACTR2 | 10097 | ACTR2 | actin related protein 2 [Source: HGNC Sym . . . |
| ACTR3 | 10096 | ACTR3 | actin related protein 3 [Source: HGNC Sym . . . |
| ADCY2 | 108 | ADCY2 | adenylate cyclase 2 [Source: HGNC Symbol; . . . |
| ADRBK1 | 156 | GRK2 | G protein-coupled receptor kinase 2 [Sou . . . |
| AKT1 | 207 | AKT1 | AKT serine/threonine kinase 1 [Source: HG . . . |
| AKT1S1 | 84335 | AKT1S1 | AKT1 substrate 1 [Source: HGNC Symbol; Acc . . . |
| AP2M1 | 1173 | AP2M1 | adaptor related protein complex 2 subuni . . . |
| ARF1 | 375 | ARF1 | ADP ribosylation factor 1 [Source: HGNC S . . . |
| ARHGDIA | 396 | ARHGDIA | Rho GDP dissociation inhibitor alpha [So . . . |
| ARPC3 | 10094 | ARPC3 | actin related protein 2/3 complex subuni . . . |
| ATF1 | 466 | ATF1 | activating transcription factor 1 [Sourc . . . |
| CAB39 | 51719 | CAB39 | calcium binding protein 39 [Source: HGNC . . . |
| CAB39L | 81617 | CAB39L | calcium binding protein 39 like [Source: . . . |
| CALR | 811 | CALR | calreticulin [Source: HGNC Symbol; Acc: HGN . . . |
| CAMK4 | 814 | CAMK4 | calcium/calmodulin dependent protein kin . . . |
| CDK1 | 983 | CDK1 | cyclin dependent kinase 1 [Source: HGNC S . . . |
| CDK2 | 1017 | CDK2 | cyclin dependent kinase 2 [Source: HGNC S . . . |
| CDK4 | 1019 | CDK4 | cyclin dependent kinase 4 [Source: HGNC S . . . |
| CDKN1A | 1026 | CDKN1A | cyclin dependent kinase inhibitor 1A [So . . . |
| CDKN1B | 1027 | CDKN1B | cyclin dependent kinase inhibitor 1B [So . . . |
| CFL1 | 1072 | CFL1 | cofilin 1 [Source: HGNC Symbol; Acc: HGNC: 1 . . . |
| CLTC | 1213 | CLTC | clathrin heavy chain [Source: HGNC Symbol . . . |
| CSNK2B | 1460 | CSNK2B | casein kinase 2 beta [Source: HGNC Symbol . . . |
| CXCR4 | 7852 | CXCR4 | C-X-C motif chemokine receptor 4 [Source . . . |
| DAPP1 | 27071 | DAPP1 | dual adaptor of phosphotyrosine and 3-ph . . . |
| DDIT3 | 1649 | DDIT3 | DNA damage inducible transcript 3 [Sourc . . . |
| DUSP3 | 1845 | DUSP3 | dual specificity phosphatase 3 [Source: H . . . |
| E2F1 | 1869 | E2F1 | E2F transcription factor 1 [Source: HGNC . . . |
| ECSIT | 51295 | ECSIT | ECSIT signaling integrator [Source: HGNC . . . |
| EGFR | 1956 | EGFR | epidermal growth factor receptor [Source . . . |
| EIF4E | 1977 | EIF4E | eukaryotic translation initiation factor . . . |
| FASLG | 356 | FASLG | Fas ligand [Source: HGNC Symbol; Acc: HGNC: . . . |
| FGF17 | 8822 | FGF17 | fibroblast growth factor 17 [Source: HGNC . . . |
| FGF22 | 27006 | FGF22 | fibroblast growth factor 22 [Source: HGNC . . . |
| FGF6 | 2251 | FGF6 | fibroblast growth factor 6 [Source: HGNC . . . |
| GNA14 | 9630 | GNA14 | G protein subunit alpha 14 [Source: HGNC . . . |
| GNGT1 | 2792 | GNGT1 | G protein subunit gamma transducin 1 [So . . . |
| GRB2 | 2885 | GRB2 | growth factor receptor bound protein 2 [ . . . |
| GSK3B | 2932 | GSK3B | glycogen synthase kinase 3 beta [Source: . . . |
| HRAS | 3265 | HRAS | “HRas proto-oncogene, GTPase [Source: HGN . . . |
| HSP90B1 | 7184 | HSP90B1 | heat shock protein 90 beta family member . . . |
| IL2RG | 3561 | IL2RG | interleukin 2 receptor subunit gamma [So . . . |
| IL4 | 3565 | IL4 | interleukin 4 [Source: HGNC Symbol; Acc: HG . . . |
| IRAK4 | 51135 | IRAK4 | interleukin 1 receptor associated kinase . . . |
| ITPR2 | 3709 | ITPR2 | “inositol 1,4,5-trisphosphate receptor t . . . |
| LCK | 3932 | LCK | “LCK proto-oncogene, Src family tyrosine . . . |
| MAP2K3 | 5606 | MAP2K3 | mitogen-activated protein kinase kinase . . . |
| MAP2K6 | 5608 | MAP2K6 | mitogen-activated protein kinase kinase . . . |
| MAP3K7 | 6885 | MAP3K7 | mitogen-activated protein kinase kinase . . . |
| MAPK1 | 5594 | MAPK1 | mitogen-activated protein kinase 1 [Sour . . . |
| MAPK10 | 5602 | MAPK10 | mitogen-activated protein kinase 10 [Sou . . . |
| MAPK8 | 5599 | MAPK8 | mitogen-activated protein kinase 8 [Sour . . . |
| MAPK9 | 5601 | MAPK9 | mitogen-activated protein kinase 9 [Sour . . . |
| MAPKAP1 | 79109 | MAPKAP1 | MAPK associated protein 1 [Source: HGNC S . . . |
| MKNK1 | 8569 | MKNK1 | MAPK interacting serine/threonine kinase . . . |
| MKNK2 | 2872 | MKNK2 | MAPK interacting serine/threonine kinase . . . |
| MYD88 | 4615 | MYD88 | MYD88 innate immune signal transduction . . . |
| NCK1 | 4690 | NCK1 | NCK adaptor protein 1 [Source: HGNC Symbo . . . |
| NFKBIB | 4793 | NFKBIB | NFKB inhibitor beta [Source: HGNC Symbol; . . . |
| NGF | 4803 | NGF | nerve growth factor [Source: HGNC Symbol; . . . |
| NOD1 | 10392 | NOD1 | nucleotide binding oligomerization domai . . . |
| PAK4 | 10298 | PAK4 | p21 (RAC1) activated kinase 4 [Source: HG . . . |
| PDK1 | 5163 | PDK1 | pyruvate dehydrogenase kinase 1 [Source: . . . |
| PFN1 | 5216 | PFN1 | profilin 1 [Source: HGNC Symbol; Acc: HGNC: . . . |
| PIK3R3 | 8503 | PIK3R3 | phosphoinositide-3-kinase regulatory sub . . . |
| PIKFYVE | 200576 | PIKFYVE | “phosphoinositide kinase, FYVE-type zinc . . . |
| PIN1 | 5300 | PIN1 | “peptidylprolyl cis/trans isomerase, NIM . . . |
| PITX2 | 5308 | PITX2 | paired like homeodomain 2 [Source: HGNC S . . . |
| PLA2G12A | 81579 | PLA2G12A | phospholipase A2 group XIIA [Source: HGNC . . . |
| PLCB1 | 23236 | PLCB1 | phospholipase C beta 1 [Source: HGNC Symb . . . |
| PLCG1 | 5335 | PLCG1 | phospholipase C gamma 1 [Source: HGNC Sym . . . |
| PPP1CA | 5499 | PPP1CA | protein phosphatase 1 catalytic subunit . . . |
| PPP2R1B | 5519 | PPP2R1B | protein phosphatase 2 scaffold subunit A . . . |
| PRKAA2 | 5563 | PRKAA2 | protein kinase AMP-activated catalytic s . . . |
| PRKAG1 | 5571 | PRKAG1 | protein kinase AMP-activated non-catalyt . . . |
| PRKAR2A | 5576 | PRKAR2A | protein kinase cAMP-dependent type Il re . . . |
| PRKCB | 5579 | PRKCB | protein kinase C beta [Source: HGNC Symbo . . . |
| PTEN | 5728 | PTEN | phosphatase and tensin homolog [Source: H . . . |
| PTPN11 | 5781 | PTPN11 | protein tyrosine phosphatase non-recepto . . . |
| RAC1 | 5879 | RAC1 | Rac family small GTPase 1 [Source: HGNC S . . . |
| RAF1 | 5894 | RAF1 | “Raf-1 proto-oncogene, serine/threonine . . . |
| RALB | 5899 | RALB | RAS like proto-oncogene B [Source: HGNC S . . . |
| RIPK1 | 8737 | RIPK1 | receptor interacting serine/threonine ki . . . |
| RIT1 | 6016 | RIT1 | Ras like without CAAX 1 [Source: HGNC Sym . . . |
| RPS6KA1 | 6195 | RPS6KA1 | ribosomal protein S6 kinase A1 [Source: H . . . |
| RPS6KA3 | 6197 | RPS6KA3 | ribosomal protein S6 kinase A3 [Source: H . . . |
| RPTOR | 57521 | RPTOR | regulatory associated protein of MTOR co . . . |
| SFN | 2810 | SFN | stratifin [Source: HGNC Symbol; Acc: HGNC: 1 . . . |
| SLA | 6503 | SLA | Src like adaptor [Source: HGNC Symbol; Acc . . . |
| SLC2A1 | 6513 | SLC2A1 | solute carrier family 2 member 1 [Source . . . |
| SMAD2 | 4087 | SMAD2 | SMAD family member 2 [Source: HGNC Symbol . . . |
| SQSTM1 | 8878 | SQSTM1 | sequestosome 1 [Source: HGNC Symbol; Acc: H . . . |
| STAT2 | 6773 | STAT2 | signal transducer and activator of trans . . . |
| TBK1 | 29110 | TBK1 | TANK binding kinase 1 [Source: HGNC Symbo . . . |
| THEM4 | 117145 | THEM4 | thioesterase superfamily member 4 [Sourc . . . |
| TIAM1 | 7074 | TIAM1 | TIAM Rac1 associated GEF 1 [Source: HGNC . . . |
| TNFRSF1A | 7132 | TNFRSF1A | TNF receptor superfamily member 1A [Sour . . . |
| TRAF2 | 7186 | TRAF2 | TNF receptor associated factor 2 [Source . . . |
| TRIB3 | 57761 | TRIB3 | tribbles pseudokinase 3 [Source: HGNC Sym . . . |
| TSC2 | 7249 | TSC2 | TSC complex subunit 2 [Source: HGNC Symbo . . . |
| UBE2D3 | 7323 | UBE2D3 | ubiquitin conjugating enzyme E2 D3 [Sour . . . |
| UBE2N | 7334 | UBE2N | ubiquitin conjugating enzyme E2 N [Sourc . . . |
| VAV3 | 10451 | VAV3 | vav guanine nucleotide exchange factor 3 . . . |
| YWHAB | 7529 | YWHAB | tyrosine 3-monooxygenase/tryptophan 5-mo . . . |

In some examples the germline variant is one or more variants of any one or more of the genes listed in table 3.

TABLE 3
Genes of M5932 (HALLMARK_INFLAMMATORY_RESPONSE).

| Original | NCBI GENE | Gene | |
|---|---|---|---|
| Member | ID | Symbol | Gene Description |
| ABCA1 | 19 | ABCA1 | ATP binding cassette subfamily A member . . . |
| ABI1 | 10006 | ABI1 | abl interactor 1 [Source: HGNC Symbol; Acc . . . |
| ACVR1B | 91 | ACVR1B | activin A receptor type 1B [Source: HGNC . . . |
| ACVR2A | 92 | ACVR2A | activin A receptor type 2A [Source: HGNC . . . |
| ADM | 133 | ADM | adrenomedullin [Source: HGNC Symbol; Acc: H . . . |
| ADORA2B | 136 | ADORA2B | adenosine A2b receptor [Source: HGNC Symb . . . |
| ADRM1 | 11047 | ADRM1 | ADRM1 26S proteasome ubiquitin receptor . . . |
| AHR | 196 | AHR | aryl hydrocarbon receptor [Source: HGNC S . . . |
| APLNR | 187 | APLNR | apelin receptor [Source: HGNC Symbol; Acc: . . . |
| AQP9 | 366 | AQP9 | aquaporin 9 [Source: HGNC Symbol; Acc: HGNC . . . |
| ATP2A2 | 488 | ATP2A2 | ATPase sarcoplasmic/endoplasmic reticulu . . . |
| ATP2B1 | 490 | ATP2B1 | ATPase plasma membrane Ca2+ transporting . . . |
| ATP2C1 | 27032 | ATP2C1 | ATPase secretory pathway Ca2+ transporti . . . |
| AXL | 558 | AXL | AXL receptor tyrosine kinase [Source: HGN . . . |
| BDKRB1 | 623 | BDKRB1 | bradykinin receptor B1 [Source: HGNC Symb . . . |
| BEST1 | 7439 | BEST1 | bestrophin 1 [Source: HGNC Symbol; Acc: HGN . . . |
| BST2 | 684 | BST2 | bone marrow stromal cell antigen 2 [Sour . . . |
| BTG2 | 7832 | BTG2 | BTG anti-proliferation factor 2 [Source: . . . |
| C3AR1 | 719 | C3AR1 | complement C3a receptor 1 [Source: HGNC S . . . |
| C5AR1 | 728 | C5AR1 | complement C5a receptor 1 [Source: HGNC S . . . |
| CALCRL | 10203 | CALCRL | calcitonin receptor like receptor [Sourc . . . |
| CCL17 | 6361 | CCL17 | C-C motif chemokine ligand 17 [Source: HG . . . |
| CCL2 | 6347 | CCL2 | C-C motif chemokine ligand 2 [Source: HGN . . . |
| CCL20 | 6364 | CCL20 | C-C motif chemokine ligand 20 [Source: HG . . . |
| CCL22 | 6367 | CCL22 | C-C motif chemokine ligand 22 [Source: HG . . . |
| CCL24 | 6369 | CCL24 | C-C motif chemokine ligand 24 [Source: HG . . . |
| CCL5 | 6352 | CCL5 | C-C motif chemokine ligand 5 [Source: HGN . . . |
| CCL7 | 6354 | CCL7 | C-C motif chemokine ligand 7 [Source: HGN . . . |
| CCR7 | 1236 | CCR7 | C-C motif chemokine receptor 7 [Source: H . . . |
| CCRL2 | 9034 | CCRL2 | C-C motif chemokine receptor like 2 [Sou . . . |
| CD14 | 929 | CD14 | CD14 molecule [Source: HGNC Symbol; Acc: HG . . . |
| CD40 | 958 | CD40 | CD40 molecule [Source: HGNC Symbol; Acc: HG . . . |
| CD48 | 962 | CD48 | CD48 molecule [Source: HGNC Symbol; Acc: HG . . . |
| CD55 | 1604 | CD55 | CD55 molecule (Cromer blood group) [Sour . . . |
| CD69 | 969 | CD69 | CD69 molecule [Source: HGNC Symbol; Acc: HG . . . |
| CD70 | 970 | CD70 | CD70 molecule [Source: HGNC Symbol; Acc: HG . . . |
| CD82 | 3732 | CD82 | CD82 molecule [Source: HGNC Symbol; Acc: HG . . . |
| CDKN1A | 1026 | CDKN1A | cyclin dependent kinase inhibitor 1A [So . . . |
| CHST2 | 9435 | CHST2 | carbohydrate sulfotransferase 2 [Source: . . . |
| CLEC5A | 23601 | CLEC5A | C-type lectin domain containing 5A [Sour . . . |
| CMKLR1 | 1240 | CMKLR1 | chemerin chemokine-like receptor 1 [Sour . . . |
| CSF1 | 1435 | CSF1 | colony stimulating factor 1 [Source: HGNC . . . |
| CSF3 | 1440 | CSF3 | colony stimulating factor 3 [Source: HGNC . . . |
| CSF3R | 1441 | CSF3R | colony stimulating factor 3 receptor [So . . . |
| CX3CL1 | 6376 | CX3CL1 | C-X3-C motif chemokine ligand 1 [Source: . . . |
| CXCL10 | 3627 | CXCL10 | C-X-C motif chemokine ligand 10 [Source: . . . |
| CXCL11 | 6373 | CXCL11 | C-X-C motif chemokine ligand 11 [Source: . . . |
| CXCL6 | 6372 | CXCL6 | C-X-C motif chemokine ligand 6 [Source: H . . . |
| CXCL9 | 4283 | CXCL9 | C-X-C motif chemokine ligand 9 [Source: H . . . |
| CXCR6 | 10663 | CXCR6 | C-X-C motif chemokine receptor 6 [Source . . . |
| CYBB | 1536 | CYBB | cytochrome b-245 beta chain [Source: HGNC . . . |
| DCBLD2 | 131566 | DCBLD2 | “discoidin, CUB and LCCL domain containi . . . |
| EBI3 | 10148 | EBI3 | Epstein-Barr virus induced 3 [Source: HGN . . . |
| EDN1 | 1906 | EDN1 | endothelin 1 [Source: HGNC Symbol; Acc: HGN . . . |
| EIF2AK2 | 5610 | EIF2AK2 | eukaryotic translation initiation factor . . . |
| EMP3 | 2014 | EMP3 | epithelial membrane protein 3 [Source: HG . . . |
| EMR1 | 2015 | ADGRE1 | adhesion G protein-coupled receptor E1 [ . . . |
| EREG | 2069 | EREG | epiregulin [Source: HGNC Symbol; Acc: HGNC: . . . |
| F3 | 2152 | F3 | “coagulation factor III, tissue factor [ . . . |
| FFAR2 | 2867 | FFAR2 | free fatty acid receptor 2 [Source: HGNC . . . |
| FPR1 | 2357 | FPR1 | formyl peptide receptor 1 [Source: HGNC S . . . |
| FZD5 | 7855 | FZD5 | frizzled class receptor 5 [Source: HGNC S . . . |
| GABBR1 | 2550 | GABBR1 | gamma-aminobutyric acid type B receptor . . . |
| GCH1 | 2643 | GCH1 | GTP cyclohydrolase 1 [Source: HGNC Symbol . . . |
| GNA15 | 2769 | GNA15 | G protein subunit alpha 15 [Source: HGNC . . . |
| GNAI3 | 2773 | GNAI3 | G protein subunit alpha i3 [Source: HGNC . . . |
| GP1BA | 2811 | GP1BA | glycoprotein lb platelet subunit alpha [ . . . |
| GPC3 | 2719 | GPC3 | glypican 3 [Source: HGNC Symbol; Acc: HGNC: . . . |
| GPR132 | 29933 | GPR132 | G protein-coupled receptor 132 [Source: H . . . |
| GPR183 | 1880 | GPR183 | G protein-coupled receptor 183 [Source: H . . . |
| HAS2 | 3037 | HAS2 | hyaluronan synthase 2 [Source: HGNC Symbo . . . |
| HBEGF | 1839 | HBEGF | heparin binding EGF like growth factor [ . . . |
| HIF1A | 3091 | HIF1A | hypoxia inducible factor 1 subunit alpha . . . |
| HPN | 3249 | HPN | hepsin [Source: HGNC Symbol; Acc: HGNC: 5155] |
| HRH1 | 3269 | HRH1 | histamine receptor H1 [Source: HGNC Symbo . . . |
| ICAM1 | 3383 | ICAM1 | intercellular adhesion molecule 1 [Sourc . . . |
| ICAM4 | 3386 | ICAM4 | intercellular adhesion molecule 4 (Lands . . . |
| ICOSLG | 23308 | ICOSLG | inducible T cell costimulator ligand [So . . . |
| IFITM1 | 8519 | IFITM1 | interferon induced transmembrane protein . . . |
| IFNAR1 | 3454 | IFNAR1 | interferon alpha and beta receptor subun . . . |
| IFNGR2 | 3460 | IFNGR2 | interferon gamma receptor 2 [Source: HGNC . . . |
| IL10 | 3586 | IL10 | interleukin 10 [Source: HGNC Symbol; Acc: H . . . |
| IL10RA | 3587 | IL10RA | interleukin 10 receptor subunit alpha [S . . . |
| IL12B | 3593 | IL12B | interleukin 12B [Source: HGNC Symbol; Acc: . . . |
| IL15 | 3600 | IL15 | interleukin 15 [Source: HGNC Symbol; Acc: H . . . |
| IL15RA | 3601 | IL15RA | interleukin 15 receptor subunit alpha [S . . . |
| IL18 | 3606 | IL18 | interleukin 18 [Source: HGNC Symbol; Acc: H . . . |
| IL18R1 | 8809 | IL18R1 | interleukin 18 receptor 1 [Source: HGNC S . . . |
| IL18RAP | 8807 | IL18RAP | interleukin 18 receptor accessory protei . . . |
| IL1A | 3552 | IL1A | interleukin 1 alpha [Source: HGNC Symbol; ... |
| IL1B | 3553 | IL1B | interleukin 1 beta [Source: HGNC Symbol; A . . . |
| IL1R1 | 3554 | IL1R1 | interleukin 1 receptor type 1 [Source: HG . . . |
| IL2RB | 3560 | IL2RB | interleukin 2 receptor subunit beta [Sou . . . |
| IL4R | 3566 | IL4R | interleukin 4 receptor [Source: HGNC Symb . . . |
| IL6 | 3569 | IL6 | interleukin 6 [Source: HGNC Symbol; Acc: HG . . . |
| IL7R | 3575 | IL7R | interleukin 7 receptor [Source: HGNC Symb . . . |
| IL8 | 3576 | CXCL8 | C-X-C motif chemokine ligand 8 [Source: H . . . |
| INHBA | 3624 | INHBA | inhibin subunit beta A [Source: HGNC Symb . . . |
| IRAK2 | 3656 | IRAK2 | interleukin 1 receptor associated kinase . . . |
| IRF1 | 3659 | IRF1 | interferon regulatory factor 1 [Source: H . . . |
| IRF7 | 3665 | IRF7 | interferon regulatory factor 7 [Source: H . . . |
| ITGA5 | 3678 | ITGA5 | integrin subunit alpha 5 [Source: HGNC Sy . . . |
| ITGB3 | 3690 | ITGB3 | integrin subunit beta 3 [Source: HGNC Sym . . . |
| ITGB8 | 3696 | ITGB8 | integrin subunit beta 8 [Source: HGNC Sym . . . |
| KCNA3 | 3738 | KCNA3 | potassium voltage-gated channel subfamil . . . |
| KCNJ2 | 3759 | KCNJ2 | potassium inwardly rectifying channel su . . . |
| KCNMB2 | 10242 | KCNMB2 | potassium calcium-activated channel subf . . . |
| KIF1B | 23095 | KIF1B | kinesin family member 1B [Source: HGNC Sy . . . |
| KLF6 | 1316 | KLF6 | Kruppel like factor 6 [Source: HGNC Symbo . . . |
| LAMP3 | 27074 | LAMP3 | lysosomal associated membrane protein 3 . . . |
| LCK | 3932 | LCK | “LCK proto-oncogene, Src family tyrosine . . . |
| LCP2 | 3937 | LCP2 | lymphocyte cytosolic protein 2 [Source: H . . . |
| LDLR | 3949 | LDLR | low density lipoprotein receptor [Source . . . |
| LIF | 3976 | LIF | LIF interleukin 6 family cytokine [Sourc . . . |
| LPAR1 | 1902 | LPAR1 | lysophosphatidic acid receptor 1 [Source . . . |
| LTA | 4049 | LTA | lymphotoxin alpha [Source: HGNC Symbol; Ac . . . |
| LY6E | 4061 | LY6E | lymphocyte antigen 6 family member E [So . . . |
| LYN | 4067 | LYN | “LYN proto-oncogene, Src family tyrosine . . . |
| MARCO | 8685 | MARCO | macrophage receptor with collagenous str . . . |
| MEFV | 4210 | MEFV | “MEFV innate immuity regulator, pyrin [S . . . |
| MEP1A | 4224 | MEP1A | meprin A subunit alpha [Source: HGNC Symb . . . |
| MET | 4233 | MET | “MET proto-oncogene, receptor tyrosine k . . . |
| MMP14 | 4323 | MMP14 | matrix metallopeptidase 14 [Source: HGNC . . . |
| MSR1 | 4481 | MSR1 | macrophage scavenger receptor 1 [Source: . . . |
| MXD1 | 4084 | MXD1 | MAX dimerization protein 1 [Source: HGNC . . . |
| MYC | 4609 | MYC | “MYC proto-oncogene, bHLH transcription . . . |
| NAMPT | 10135 | NAMPT | nicotinamide phosphoribosyltransferase [ . . . |
| NDP | 4693 | NDP | norrin cystine knot growth factor NDP [S . . . |
| NFKB1 | 4790 | NFKB1 | nuclear factor kappa B subunit 1 [Source . . . |
| NFKBIA | 4792 | NFKBIA | NFKB inhibitor alpha [Source: HGNC Symbol . . . |
| NLRP3 | 114548 | NLRP3 | NLR family pyrin domain containing 3 [So . . . |
| NMI | 9111 | NMI | N-myc and STAT interactor [Source: HGNC S . . . |
| NMUR1 | 10316 | NMUR1 | neuromedin U receptor 1 [Source: HGNC Sym . . . |
| NOD2 | 64127 | NOD2 | nucleotide binding oligomerization domai . . . |
| NPFFR2 | 10886 | NPFFR2 | neuropeptide FF receptor 2 [Source: HGNC . . . |
| OLR1 | 4973 | OLR1 | oxidized low density lipoprotein recepto . . . |
| OPRK1 | 4986 | OPRK1 | opioid receptor kappa 1 [Source: HGNC Sym . . . |
| OSM | 5008 | OSM | oncostatin M [Source: HGNC Symbol; Acc: HGN . . . |
| OSMR | 9180 | OSMR | oncostatin M receptor [Source: HGNC Symbo . . . |
| P2RX4 | 5025 | P2RX4 | purinergic receptor P2X 4 [Source: HGNC S . . . |
| P2RX7 | 5027 | P2RX7 | purinergic receptor P2X 7 [Source: HGNC S . . . |
| P2RY2 | 5029 | P2RY2 | purinergic receptor P2Y2 [Source: HGNC Sy . . . |
| PCDH7 | 5099 | PCDH7 | protocadherin 7 [Source: HGNC Symbol; Acc: . . . |
| PDE4B | 5142 | PDE4B | phosphodiesterase 4B [Source: HGNC Symbol . . . |
| PDPN | 10630 | PDPN | podoplanin [Source: HGNC Symbol; Acc: HGNC: . . . |
| PIK3R5 | 23533 | PIK3R5 | phosphoinositide-3-kinase regulatory sub . . . |
| PLAUR | 5329 | PLAUR | “plasminogen activator, urokinase recept . . . |
| PROK2 | 60675 | PROK2 | prokineticin 2 [Source: HGNC Symbol; Acc: H . . . |
| PSEN1 | 5663 | PSEN1 | presenilin 1 [Source: HGNC Symbol; Acc: HGN . . . |
| PTAFR | 5724 | PTAFR | platelet activating factor receptor [Sou . . . |
| PTGER2 | 5732 | PTGER2 | prostaglandin E receptor 2 [Source: HGNC . . . |
| PTGER4 | 5734 | PTGER4 | prostaglandin E receptor 4 [Source: HGNC . . . |
| PTGIR | 5739 | PTGIR | prostaglandin 12 receptor [Source: HGNC S . . . |
| PTPRE | 5791 | PTPRE | protein tyrosine phosphatase receptor ty . . . |
| PVR | 5817 | PVR | PVR cell adhesion molecule [Source: HGNC . . . |
| RAF1 | 5894 | RAF1 | “Raf-1 proto-oncogene, serine/threonine . . . |
| RASGRP1 | 10125 | RASGRP1 | RAS guanyl releasing protein 1 [Source: H . . . |
| RELA | 5970 | RELA | “RELA proto-oncogene, NF-KB subunit [Sou . . . |
| RGS1 | 5996 | RGS1 | regulator of G protein signaling 1 [Sour . . . |
| RGS16 | 6004 | RGS16 | regulator of G protein signaling 16 [Sou . . . |
| RHOG | 391 | RHOG | ras homolog family member G [Source: HGNC . . . |
| RIPK2 | 8767 | RIPK2 | receptor interacting serine/threonine ki . . . |
| RNF144B | 255488 | RNF144B | ring finger protein 144B [Source: HGNC Sy . . . |
| ROS1 | 6098 | ROS1 | “ROS proto-oncogene 1, receptor tyrosine . . . |
| RTP4 | 64108 | RTP4 | receptor transporter protein 4 [Source: H . . . |
| SCARF1 | 8578 | SCARF1 | scavenger receptor class F member 1 [Sou . . . |
| SCN1B | 6324 | SCN1B | sodium voltage-gated channel beta subuni . . . |
| SELE | 6401 | SELE | selectin E [Source: HGNC Symbol; Acc: HGNC: . . . |
| SELL | 6402 | SELL | selectin L [Source: HGNC Symbol; Acc: HGNC: . . . |
| SELS | 55829 | SELENOS | selenoprotein S [Source: HGNC Symbol; Acc: . . . |
| SEMA4D | 10507 | SEMA4D | semaphorin 4D [Source: HGNC Symbol; Acc: HG . . . |
| SERPINE1 | 5054 | SERPINE1 | serpin family E member 1 [Source: HGNC Sy . . . |
| SGMS2 | 166929 | SGMS2 | sphingomyelin synthase 2 [Source: HGNC Sy . . . |
| SLAMF1 | 6504 | SLAMF1 | signaling lymphocytic activation molecul . . . |
| SLC11A2 | 4891 | SLC11A2 | solute carrier family 11 member 2 [Sourc . . . |
| SLC1A2 | 6506 | SLC1A2 | solute carrier family 1 member 2 [Source . . . |
| SLC28A2 | 9153 | SLC28A2 | solute carrier family 28 member 2 [Sourc . . . |
| SLC31A1 | 1317 | SLC31A1 | solute carrier family 31 member 1 [Sourc . . . |
| SLC31A2 | 1318 | SLC31A2 | solute carrier family 31 member 2 [Sourc . . . |
| SLC4A4 | 8671 | SLC4A4 | solute carrier family 4 member 4 [Source . . . |
| SLC7A1 | 6541 | SLC7A1 | solute carrier family 7 member 1 [Source . . . |
| SLC7A2 | 6542 | SLC7A2 | solute carrier family 7 member 2 [Source . . . |
| SPHK1 | 8877 | SPHK1 | sphingosine kinase 1 [Source: HGNC Symbol . . . |
| SRI | 6717 | SRI | sorcin [Source: HGNC Symbol; Acc: HGNC: 11292] |
| STAB1 | 23166 | STAB1 | stabilin 1 [Source: HGNC Symbol; Acc: HGNC: . . . |
| TACR1 | 6869 | TACR1 | tachykinin receptor 1 [Source: HGNC Symbo . . . |
| TACR3 | 6870 | TACR3 | tachykinin receptor 3 [Source: HGNC Symbo . . . |
| TAPBP | 6892 | TAPBP | TAP binding protein [Source: HGNC Symbol ; . . . |
| TIMP1 | 7076 | TIMP1 | TIMP metallopeptidase inhibitor 1 [Sourc . . . |
| TLR1 | 7096 | TLR1 | toll like receptor 1 [Source: HGNC Symbol . . . |
| TLR2 | 7097 | TLR2 | toll like receptor 2 [Source: HGNC Symbol . . . |
| TLR3 | 7098 | TLR3 | toll like receptor 3 [Source: HGNC Symbol . . . |
| TNFAIP6 | 7130 | TNFAIP6 | TNF alpha induced protein 6 [Source: HGNC . . . |
| TNFRSF1B | 7133 | TNFRSF1B | TNF receptor superfamily member 1B [Sour . . . |
| TNFRSF9 | 3604 | TNFRSF9 | TNF receptor superfamily member 9 [Sourc . . . |
| TNFSF10 | 8743 | TNFSF10 | TNF superfamily member 10 [Source: HGNC S . . . |
| TNFSF15 | 9966 | TNFSF15 | TNF superfamily member 15 [Source: HGNC S . . . |
| TNFSF9 | 8744 | TNFSF9 | TNF superfamily member 9 [Source: HGNC Sy . . . |
| TPBG | 7162 | TPBG | trophoblast glycoprotein [Source: HGNC Sy . . . |
| VIP | 7432 | VIP | vasoactive intestinal peptide [Source: HG . . . |

In some examples the germline variant is one or more variants of any one or more of the genes listed in table 4.

TABLE 4
Genes of M5953 (HALLMARK_KRAS_SIGNALING_UP).

| Original | NCBI GENE | Gene | |
|---|---|---|---|
| Member | ID | Symbol | Gene Description |
| PDCD1LG2 | 80380 | PDCD1LG2 | programmed cell death 1 ligand 2 [Sourc . . . |
| PECAM1 | 5175 | PECAM1 | platelet and endothelial cell adhesion . . . |
| PEG3 | 5178 | PEG3 | paternally expressed 3 [Source: HGNC Sym . . . |
| PIGR | 5284 | PIGR | polymeric immunoglobulin receptor [Sour . . . |
| PLAT | 5327 | PLAT | “plasminogen activator, tissue type [So . . . |
| PLAU | 5328 | PLAU | “plasminogen activator, urokinase [Sour . . . |
| PLAUR | 5329 | PLAUR | “plasminogen activator, urokinase recep . . . |
| PLEK2 | 26499 | PLEK2 | pleckstrin 2 [Source: HGNC Symbol; Acc: HG . . . |
| PLVAP | 83483 | PLVAP | plasmalemma vesicle associated protein . . . |
| PPBP | 5473 | PPBP | pro-platelet basic protein [Source: HGNC . . . |
| PPP1R15A | 23645 | PPP1R15A | protein phosphatase 1 regulatory subuni . . . |
| PRDM1 | 639 | PRDM1 | PR/SET domain 1 [Source: HGNC Symbol; Acc . . . |
| PRKG2 | 5593 | PRKG2 | protein kinase cGMP-dependent 2 [Source . . . |
| PRRX1 | 5396 | PRRX1 | paired related homeobox 1 [Source: HGNC . . . |
| PSMB8 | 5696 | PSMB8 | proteasome 20S subunit beta 8 [Source: H . . . |
| PTBP2 | 58155 | PTBP2 | polypyrimidine tract binding protein 2 . . . |
| PTCD2 | 79810 | PTCD2 | pentatricopeptide repeat domain 2 [Sour . . . |
| PTGS2 | 5743 | PTGS2 | prostaglandin-endoperoxide synthase 2 [ . . . |
| PTPRR | 5801 | PTPRR | protein tyrosine phosphatase receptor t . . . |
| RABGAP1L | 9910 | RABGAP1L | RAB GTPase activating protein 1 like [S . . . |
| RBM4 | 5936 | RBM4 | RNA binding motif protein 4 [Source: HGN . . . |
| RBP4 | 5950 | RBP4 | retinol binding protein 4 [Source: HGNC . . . |
| RELN | 5649 | RELN | reelin [Source: HGNC Symbol; Acc: HGNC: 9957] |
| RETN | 56729 | RETN | resistin [Source: HGNC Symbol; Acc: HGNC: 2 . . . |
| RGS16 | 6004 | RGS16 | regulator of G protein signaling 16 [So . . . |
| SATB1 | 6304 | SATB1 | SATB homeobox 1 [Source: HGNC Symbol; Acc . . . |
| SCG3 | 29106 | SCG3 | secretogranin III [Source: HGNC Symbol; A . . . |
| SCG5 | 6447 | SCG5 | secretogranin V [Source: HGNC Symbol; Acc . . . |
| SCN1B | 6324 | SCN1B | sodium voltage-gated channel beta subun . . . |
| SDCCAG8 | 10806 | SDCCAG8 | SHH signaling and ciliogenesis regulato . . . |
| SEMA3B | 7869 | SEMA3B | semaphorin 3B [Source: HGNC Symbol; Acc: H . . . |
| SERPINA3 | 12 | SERPINA3 | serpin family A member 3 [Source: HGNC S . . . |
| SLMO2 | 51012 | PRELID3B | PRELI domain containing 3B [Source: HGNC . . . |
| SLPI | 6590 | SLPI | secretory leukocyte peptidase inhibitor . . . |
| SNAP25 | 6616 | SNAP25 | synaptosome associated protein 25 [Sour . . . |
| SNAP91 | 9892 | SNAP91 | synaptosome associated protein 91 [Sour . . . |
| SOX9 | 6662 | SOX9 | SRY-box transcription factor 9 [Source: . . . |
| SPARCL1 | 8404 | SPARCL1 | SPARC like 1 [Source: HGNC Symbol; Acc: HG . . . |
| SPON1 | 10418 | SPON1 | spondin 1 [Source: HGNC Symbol; Acc: HGNC: . . . |
| SPP1 | 6696 | SPP1 | secreted phosphoprotein 1 [Source: HGNC . . . |
| SPRY2 | 10253 | SPRY2 | sprouty RTK signaling antagonist 2 [Sou . . . |
| ST6GAL1 | 6480 | ST6GAL1 | “ST6 beta-galactoside alpha-2,6-sialylt . . . |
| STRN | 6801 | STRN | striatin [Source: HGNC Symbol; Acc: HGNC: 1 . . . |
| TFPI | 7035 | TFPI | tissue factor pathway inhibitor [Source . . . |
| TLR8 | 51311 | TLR8 | toll like receptor 8 [Source: HGNC Symbo . . . |
| TMEM100 | 55273 | TMEM100 | transmembrane protein 100 [Source: HGNC . . . |
| TMEM158 | 25907 | TMEM158 | transmembrane protein 158 [Source: HGNC . . . |
| TMEM176A | 55365 | TMEM176A | transmembrane protein 176A [Source: HGNC . . . |
| TMEM176B | 28959 | TMEM176B | transmembrane protein 176B [Source: HGNC . . . |
| TNFAIP3 | 7128 | TNFAIP3 | TNF alpha induced protein 3 [Source: HGN . . . |
| TNFRSF1B | 7133 | TNFRSF1B | TNF receptor superfamily member 1B [Sou . . . |
| TNNT2 | 7139 | TNNT2 | “troponin T2, cardiac type [Source: HGNC . . . |
| TOR1AIP2 | 163590 | TOR1AIP2 | torsin 1A interacting protein 2 [Source . . . |
| TPH1 | 7166 | TPH1 | tryptophan hydroxylase 1 [Source: HGNC S . . . |
| TRAF1 | 7185 | TRAF1 | TNF receptor associated factor 1 [Sourc . . . |
| TRIB1 | 10221 | TRIB1 | tribbles pseudokinase 1 [Source: HGNC Sy . . . |
| TRIB2 | 28951 | TRIB2 | tribbles pseudokinase 2 [Source: HGNC Sy . . . |
| TSPAN1 | 10103 | TSPAN1 | tetraspanin 1 [Source: HGNC Symbol; Acc: H . . . |
| TSPAN13 | 27075 | TSPAN13 | tetraspanin 13 [Source: HGNC Symbol; Acc: . . . |
| TSPAN7 | 7102 | TSPAN7 | tetraspanin 7 [Source: HGNC Symbol; Acc: H . . . |
| USH1C | 10083 | USH1C | USH1 protein network component harmonin . . . |
| USP12 | 219333 | USP12 | ubiquitin specific peptidase 12 [Source . . . |
| VWA5A | 4013 | VWA5A | von Willebrand factor A domain containi . . . |
| WDR33 | 55339 | WDR33 | WD repeat domain 33 [Source: HGNC Symbol . . . |
| WNT7A | 7476 | WNT7A | Wnt family member 7A [Source: HGNC |
| | | | Symbo . . . |
| YRDC | 79693 | YRDC | yrdC N6-threonylcarbamoyltransferase do . . . |
| ZNF277 | 11179 | ZNF277 | zinc finger protein 277 [Source: HGNC Sy . . . |
| ZNF639 | 51193 | ZNF639 | zinc finger protein 639 [Source: HGNC Sy . . . |

In some examples the germline variant is one or more variants of any one or more of the genes listed in table 5.

TABLE 5
Genes of M5957 (HALLMARK_PANCREAS_BETA_CELLS).

| Original | NCBI GENE | | |
|---|---|---|---|
| Member | ID | Gene Symbol | Gene Description |
| ABCC8 | 6833 | ABCC8 | ATP binding cassette subfamily C member 8 [ . . . |
| AKT3 | 10000 | AKT3 | AKT serine/threonine kinase 3 [Source: HGNC . . . |
| CHGA | 1113 | CHGA | chromogranin A [Source: HGNC |
| | | | Symbol; Acc: HGNC . . . |
| DCX | 1641 | DCX | doublecortin [Source: HGNC |
| | | | Symbol; Acc: HGNC: 2 . . . |
| DPP4 | 1803 | DPP4 | dipeptidyl peptidase 4 [Source: HGNC Symbol ; . . . |
| ELP4 | 26610 | ELP4 | elongator acetyltransferase complex subunit . . . |
| FOXA2 | 3170 | FOXA2 | forkhead box A2 [Source: HGNC |
| | | | Symbol; Acc: HGN . . . |
| FOXO1 | 2308 | FOXO1 | forkhead box O1 [Source: HGNC |
| | | | Symbol; Acc: HGN . . . |
| G6PC2 | 57818 | G6PC2 | glucose-6-phosphatase catalytic subunit 2 [ . . . |
| GCG | 2641 | GCG | glucagon [Source: HGNC Symbol; Acc: HGNC: 4191] |
| GCK | 2645 | GCK | glucokinase [Source: HGNC |
| | | | Symbol; Acc: HGNC: 4195] |
| HNF1A | 6927 | HNF1A | HNF1 homeobox A [Source: HGNC |
| | | | Symbol; Acc: HGN . . . |
| IAPP | 3375 | IAPP | islet amyloid polypeptide [Source: HGNC Symb . . . |
| INS | 3630 | INS | insulin [Source: HGNC Symbol; Acc: HGNC: 6081] |
| INSM1 | 3642 | INSM1 | INSM transcriptional repressor 1 [Source: HG . . . |
| ISL1 | 3670 | ISL1 | ISL LIM homeobox 1 [Source: HGNC |
| | | | Symbol; Acc: . . . |
| LMO2 | 4005 | LMO2 | LIM domain only 2 [Source: HGNC |
| | | | Symbol; Acc: H . . . |
| MAFB | 9935 | MAFB | MAF bZIP transcription factor B [Source: HGN . . . |
| NEUROD1 | 4760 | NEUROD1 | neuronal differentiation 1 [Source: HGNC Sym . . . |
| NEUROG3 | 50674 | NEUROG3 | neurogenin 3 [Source: HGNC |
| | | | Symbol; Acc: HGNC: 1 . . . |
| NKX2-2 | 4821 | NKX2-2 | NK2 homeobox 2 [Source: HGNC |
| | | | Symbol; Acc: HGNC . . . |
| NKX6-1 | 4825 | NKX6-1 | NK6 homeobox 1 [Source: HGNC |
| | | | Symbol; Acc: HGNC . . . |
| PAK3 | 5063 | PAK3 | p21 (RAC1) activated kinase 3 [Source: HGNC . . . |
| PAX4 | 5078 | PAX4 | paired box 4 [Source: HGNC |
| | | | Symbol; Acc: HGNC: 8 . . . |
| PAX6 | 5080 | PAX6 | paired box 6 [Source: HGNC |
| | | | Symbol; Acc: HGNC: 8 . . . |
| PCSK1 | 5122 | PCSK1 | proprotein convertase subtilisin/kexin type . . . |
| PCSK2 | 5126 | PCSK2 | proprotein convertase subtilisin/kexin type . . . |
| PDX1 | 3651 | PDX1 | pancreatic and duodenal homeobox 1 [Source: . . . |
| PKLR | 5313 | PKLR | pyruvate kinase L/R [Source: HGNC Symbol; Acc . . . |
| SCGN | 10590 | SCGN | “secretagogin, EF-hand calcium binding prot . . . |
| SEC11A | 23478 | SEC11A | “SEC11 homolog A, signal peptidase complex . . . |
| SLC2A2 | 6514 | SLC2A2 | solute carrier family 2 member 2 [Source: HG . . . |
| SPCS1 | 28972 | SPCS1 | signal peptidase complex subunit 1 [Source: . . . |
| SRP14 | 6727 | SRP14 | signal recognition particle 14 [Source: HGNC . . . |
| SRP9 | 6726 | SRP9 | signal recognition particle 9 [Source: HGNC . . . |
| SRPRB | 58477 | SRPRB | SRP receptor subunit beta [Source: HGNC Symb . . . |
| SST | 6750 | SST | somatostatin [Source: HGNC |
| | | | Symbol; Acc: HGNC: 1 . . . |
| STXBP1 | 6812 | STXBP1 | syntaxin binding protein 1 [Source: HGNC Sym . . . |
| SYT13 | 57586 | SYT13 | synaptotagmin 13 [Source: HGNC |
| | | | Symbol; Acc: HG . . . |
| VDR | 7421 | VDR | vitamin D receptor [Source: HGNC Symbol; Acc: . . . |

In some examples the germline variant is one or more variants of any one or more of the genes listed in table 6.

TABLE 6
genes of M5890 (HALLMARK_TNFA_SIGNALING_VIA_NFKB).

| Original | NCBI | Gene | |
|---|---|---|---|
| Member | GENE ID | Symbol | Gene Description |
| ABCA1 | 19 | ABCA1 | ATP binding cassette subfamily A member . . . |
| AREG | 374 | AREG | amphiregulin [Source: HGNC Symbol; Acc: HGN . . . |
| ATF3 | 467 | ATF3 | activating transcription factor 3 [Sourc . . . |
| ATP2B1 | 490 | ATP2B1 | ATPase plasma membrane Ca2+ transporting . . . |
| B4GALT1 | 2683 | B4GALT1 | “beta-1,4-galactosyltransferase 1 [Sourc . . . |
| B4GALT5 | 9334 | B4GALT5 | “beta-1,4-galactosyltransferase 5 [Sourc . . . |
| BCL2A1 | 597 | BCL2A1 | BCL2 related protein A1 [Source: HGNC Sym . . . |
| BCL3 | 602 | BCL3 | BCL3 transcription coactivator [Source: H . . . |
| BCL6 | 604 | BCL6 | BCL6 transcription repressor [Source: HGN . . . |
| BHLHE40 | 8553 | BHLHE40 | basic helix-loop-helix family member e40 . . . |
| BIRC2 | 329 | BIRC2 | baculoviral IAP repeat containing 2 [Sou . . . |
| BIRC3 | 330 | BIRC3 | baculoviral IAP repeat containing 3 [Sou . . . |
| BMP2 | 650 | BMP2 | bone morphogenetic protein 2 [Source: HGN . . . |
| BTG1 | 694 | BTG1 | BTG anti-proliferation factor 1 [Source: . . . |
| BTG2 | 7832 | BTG2 | BTG anti-proliferation factor 2 [Source: . . . |
| BTG3 | 10950 | BTG3 | BTG anti-proliferation factor 3 [Source: . . . |
| CCL2 | 6347 | CCL2 | C-C motif chemokine ligand 2 [Source: HGN . . . |
| CCL20 | 6364 | CCL20 | C-C motif chemokine ligand 20 [Source: HG . . . |
| CCL4 | 6351 | CCL4 | C-C motif chemokine ligand 4 [Source: HGN . . . |
| CCL5 | 6352 | CCL5 | C-C motif chemokine ligand 5 [Source: HGN . . . |
| CCND1 | 595 | CCND1 | cyclin D1 [Source: HGNC Symbol; Acc: HGNC: 1 . . . |
| CCNL1 | 57018 | CCNL1 | cyclin L1 [Source: HGNC Symbol; Acc: HGNC: 2 . . . |
| CCRL2 | 9034 | CCRL2 | C-C motif chemokine receptor like 2 [Sou . . . |
| CD44 | 960 | CD44 | CD44 molecule (Indian blood group) [Sour . . . |
| CD69 | 969 | CD69 | CD69 molecule [Source: HGNC Symbol; Acc: HG . . . |
| CD80 | 941 | CD80 | CD80 molecule [Source: HGNC Symbol; Acc: HG . . . |
| CD83 | 9308 | CD83 | CD83 molecule [Source: HGNC Symbol; Acc: HG . . . |
| CDKN1A | 1026 | CDKN1A | cyclin dependent kinase inhibitor 1A [So . . . |
| CEBPB | 1051 | CEBPB | CCAAT enhancer binding protein beta [Sou . . . |
| CEBPD | 1052 | CEBPD | CCAAT enhancer binding protein delta [So . . . |
| CFLAR | 8837 | CFLAR | CASP8 and FADD like apoptosis regulator . . . |
| CLCF1 | 23529 | CLCF1 | cardiotrophin like cytokine factor 1 [So . . . |
| CSF1 | 1435 | CSF1 | colony stimulating factor 1 [Source: HGNC . . . |
| CSF2 | 1437 | CSF2 | colony stimulating factor 2 [Source: HGNC . . . |
| CXCL1 | 2919 | CXCL1 | C-X-C motif chemokine ligand 1 [Source: H . . . |
| CXCL10 | 3627 | CXCL10 | C-X-C motif chemokine ligand 10 [Source: . . . |
| CXCL11 | 6373 | CXCL11 | C-X-C motif chemokine ligand 11 [Source: . . . |
| CXCL2 | 2920 | CXCL2 | C-X-C motif chemokine ligand 2 [Source: H . . . |
| CXCL3 | 2921 | CXCL3 | C-X-C motif chemokine ligand 3 [Source: H . . . |
| CXCL6 | 6372 | CXCL6 | C-X-C motif chemokine ligand 6 [Source: H . . . |
| CXCR7 | 57007 | ACKR3 | atypical chemokine receptor 3 [Source: HG . . . |
| CYR61 | 3491 | CCN1 | cellular communication network factor 1 . . . |
| DDX58 | 23586 | DDX58 | DExD/H-box helicase 58 [Source: HGNC Symb . . . |
| DENND5A | 23258 | DENND5A | DENN domain containing 5A [Source: HGNC S . . . |
| DNAJB4 | 11080 | DNAJB4 | DnaJ heat shock protein family (Hsp40) m . . . |
| DRAM1 | 55332 | DRAM1 | DNA damage regulated autophagy modulator . . . |
| DUSP1 | 1843 | DUSP1 | dual specificity phosphatase 1 [Source: H . . . |
| DUSP2 | 1844 | DUSP2 | dual specificity phosphatase 2 [Source: H . . . |
| DUSP4 | 1846 | DUSP4 | dual specificity phosphatase 4 [Source: H . . . |
| DUSP5 | 1847 | DUSP5 | dual specificity phosphatase 5 [Source: H . . . |
| EDN1 | 1906 | EDN1 | endothelin 1 [Source: HGNC Symbol; Acc: HGN . . . |
| EFNA1 | 1942 | EFNA1 | ephrin A1 [Source: HGNC Symbol; Acc: HGNC: 3 . . . |
| EGR1 | 1958 | EGR1 | early growth response 1 [Source: HGNC Sym . . . |
| EGR2 | 1959 | EGR2 | early growth response 2 [Source: HGNC Sym . . . |
| EGR3 | 1960 | EGR3 | early growth response 3 [Source: HGNC Sym . . . |
| EHD1 | 10938 | EHD1 | EH domain containing 1 [Source: HGNC Symb . . . |
| EIF1 | 10209 | EIF1 | eukaryotic translation initiation factor . . . |
| ETS2 | 2114 | ETS2 | “ETS proto-oncogene 2, transcription fac . . . |
| F2RL1 | 2150 | F2RL1 | F2R like trypsin receptor 1 [Source: HGNC . . . |
| F3 | 2152 | F3 | “coagulation factor III, tissue factor [ . . . |
| FJX1 | 24147 | FJX1 | four-jointed box kinase 1 [Source: HGNC S . . . |
| FOS | 2353 | FOS | “Fos proto-oncogene, AP-1 transcription . . . |
| FOSB | 2354 | FOSB | “FosB proto-oncogene, AP-1 transcription . . . |
| FOSL1 | 8061 | FOSL1 | “FOS like 1, AP-1 transcription factor s . . . |
| FOSL2 | 2355 | FOSL2 | “FOS like 2, AP-1 transcription factor s . . . |
| FUT4 | 2526 | FUT4 | fucosyltransferase 4 [Source: HGNC Symbol . . . |
| GOS2 | 50486 | GOS2 | G0/G1 switch 2 [Source: HGNC Symbol; Acc: H . . . |
| GADD45A | 1647 | GADD45A | growth arrest and DNA damage inducible a . . . |
| GADD45B | 4616 | GADD45B | growth arrest and DNA damage inducible b . . . |
| GCH1 | 2643 | GCH1 | GTP cyclohydrolase 1 [Source: HGNC Symbol . . . |
| GEM | 2669 | GEM | GTP binding protein overexpressed in ske . . . |
| GFPT2 | 9945 | GFPT2 | glutamine-fructose-6-phosphate transamin . . . |
| GPR183 | 1880 | GPR183 | G protein-coupled receptor 183 [Source: H . . . |
| HBEGF | 1839 | HBEGF | heparin binding EGF like growth factor [ . . . |
| HES1 | 3280 | HES1 | hes family bHLH transcription factor 1 [ . . . |
| ICAM1 | 3383 | ICAM1 | intercellular adhesion molecule 1 [Sourc . . . |
| ICOSLG | 23308 | ICOSLG | inducible T cell costimulator ligand [So . . . |
| ID2 | 3398 | ID2 | inhibitor of DNA binding 2 [Source: HGNC . . . |
| IER2 | 9592 | IER2 | immediate early response 2 [Source: HGNC . . . |
| IER3 | 8870 | IER3 | immediate early response 3 [Source: HGNC . . . |
| IER5 | 51278 | IER5 | immediate early response 5 [Source: HGNC . . . |
| IFIH1 | 64135 | IFIH1 | interferon induced with helicase C domai . . . |
| IFIT2 | 3433 | IFIT2 | interferon induced protein with tetratri . . . |
| IFNGR2 | 3460 | IFNGR2 | interferon gamma receptor 2 [Source: HGNC . . . |
| IL12B | 3593 | IL12B | interleukin 12B [Source: HGNC Symbol; Acc: . . . |
| IL15RA | 3601 | IL15RA | interleukin 15 receptor subunit alpha [S . . . |
| IL18 | 3606 | IL18 | interleukin 18 [Source: HGNC Symbol; Acc: H . . . |
| IL1A | 3552 | IL1A | interleukin 1 alpha [Source: HGNC Symbol ; . . . |
| IL1B | 3553 | IL1B | interleukin 1 beta [Source: HGNC Symbol; A . . . |
| IL23A | 51561 | IL23A | interleukin 23 subunit alpha [Source: HGN . . . |
| IL6 | 3569 | IL6 | interleukin 6 [Source: HGNC Symbol; Acc: HG . . . |
| IL6ST | 3572 | IL6ST | interleukin 6 cytokine family signal tra . . . |
| IL7R | 3575 | IL7R | interleukin 7 receptor [Source: HGNC Symb . . . |
| INHBA | 3624 | INHBA | inhibin subunit beta A [Source: HGNC Symb . . . |
| IRF1 | 3659 | IRF1 | interferon regulatory factor 1 [Source: H . . . |
| IRS2 | 8660 | IRS2 | insulin receptor substrate 2 [Source: HGN . . . |
| JAG1 | 182 | JAG1 | jagged canonical Notch ligand 1 [Source: . . . |
| JUN | 3725 | JUN | “Jun proto-oncogene, AP-1 transcription . . . |
| JUNB | 3726 | JUNB | “JunB proto-oncogene, AP-1 transcription . . . |
| KDM6B | 23135 | KDM6B | lysine demethylase 6B [Source: HGNC Symbo . . . |
| KLF10 | 7071 | KLF10 | Kruppel like factor 10 [Source: HGNC Symb . . . |
| KLF2 | 10365 | KLF2 | Kruppel like factor 2 [Source: HGNC Symbo . . . |
| KLF4 | 9314 | KLF4 | Kruppel like factor 4 [Source: HGNC Symbo . . . |
| KLF6 | 1316 | KLF6 | Kruppel like factor 6 [Source: HGNC Symbo . . . |
| KLF9 | 687 | KLF9 | Kruppel like factor 9 [Source: HGNC Symbo . . . |
| KYNU | 8942 | KYNU | kynureninase [Source: HGNC Symbol; Acc: HGN . . . |
| LAMB3 | 3914 | LAMB3 | laminin subunit beta 3 [Source: HGNC Symb . . . |
| LDLR | 3949 | LDLR | low density lipoprotein receptor [Source . . . |
| LIF | 3976 | LIF | LIF interleukin 6 family cytokine [Sourc . . . |
| LITAF | 9516 | LITAF | lipopolysaccharide induced TNF factor [S . . . |
| MAFF | 23764 | MAFF | MAF bZIP transcription factor F [Source: . . . |
| MAP2K3 | 5606 | MAP2K3 | mitogen-activated protein kinase kinase . . . |
| MAP3K8 | 1326 | MAP3K8 | mitogen-activated protein kinase kinase . . . |
| MARCKS | 4082 | MARCKS | myristoylated alanine rich protein kinas . . . |
| MCL1 | 4170 | MCL1 | “MCL1 apoptosis regulator, BCL2 family m . . . |
| MSC | 9242 | MSC | musculin [Source: HGNC Symbol; Acc: HGNC: 7321] |
| MXD1 | 4084 | MXD1 | MAX dimerization protein 1 [Source: HGNC . . . |
| MYC | 4609 | MYC | “MYC proto-oncogene, bHLH transcription . . . |
| NAMPT | 10135 | NAMPT | nicotinamide phosphoribosyltransferase [ . . . |
| NFAT5 | 10725 | NFAT5 | nuclear factor of activated T cells 5 [S . . . |
| NFE2L2 | 4780 | NFE2L2 | “nuclear factor, erythroid 2 like 2 [Sou . . . |
| NFIL3 | 4783 | NFIL3 | “nuclear factor, interleukin 3 regulated . . . |
| NFKB1 | 4790 | NFKB1 | nuclear factor kappa B subunit 1 [Source . . . |
| NFKB2 | 4791 | NFKB2 | nuclear factor kappa B subunit 2 [Source . . . |
| NFKBIA | 4792 | NFKBIA | NFKB inhibitor alpha [Source: HGNC Symbol . . . |
| NFKBIE | 4794 | NFKBIE | NFKB inhibitor epsilon [Source: HGNC Symb . . . |
| NINJ1 | 4814 | NINJ1 | ninjurin 1 [Source: HGNC Symbol; Acc: HGNC: . . . |
| NR4A1 | 3164 | NR4A1 | nuclear receptor subfamily 4 group A mem . . . |
| NR4A2 | 4929 | NR4A2 | nuclear receptor subfamily 4 group A mem . . . |
| NR4A3 | 8013 | NR4A3 | nuclear receptor subfamily 4 group A mem . . . |
| OLR1 | 4973 | OLR1 | oxidized low density lipoprotein recepto . . . |
| PANX1 | 24145 | PANX1 | pannexin 1 [Source: HGNC Symbol; Acc: HGNC: . . . |
| PDE4B | 5142 | PDE4B | phosphodiesterase 4B [Source: HGNC Symbol . . . |
| PDLIM5 | 10611 | PDLIM5 | PDZ and LIM domain 5 [Source: HGNC Symbol . . . |
| PER1 | 5187 | PER1 | period circadian regulator 1 [Source: HGN . . . |
| PFKFB3 | 5209 | PFKFB3 | “6-phosphofructo-2-kinase/fructose-2,6-b . . . |
| PHLDA1 | 22822 | PHLDA1 | pleckstrin homology like domain family A . . . |
| PHLDA2 | 7262 | PHLDA2 | pleckstrin homology like domain family A . . . |
| PLAU | 5328 | PLAU | “plasminogen activator, urokinase [Sourc . . . |
| PLAUR | 5329 | PLAUR | “plasminogen activator, urokinase recept . . . |
| PLEK | 5341 | PLEK | pleckstrin [Source: HGNC Symbol; Acc: HGNC: . . . |
| PLK2 | 10769 | PLK2 | polo like kinase 2 [Source: HGNC Symbol; A . . . |
| PMEPA1 | 56937 | PMEPA1 | “prostate transmembrane protein, androge . . . |
| PNRC1 | 10957 | PNRC1 | proline rich nuclear receptor coactivato . . . |
| PPAP2B | 8613 | PLPP3 | phospholipid phosphatase 3 [Source: HGNC . . . |
| PPP1R15A | 23645 | PPP1R15A | protein phosphatase 1 regulatory subunit . . . |
| PTGER4 | 5734 | PTGER4 | prostaglandin E receptor 4 [Source: HGNC . . . |
| PTGS2 | 5743 | PTGS2 | prostaglandin-endoperoxide synthase 2 [S . . . |
| PTPRE | 5791 | PTPRE | protein tyrosine phosphatase receptor ty . . . |
| PTX3 | 5806 | PTX3 | pentraxin 3 [Source: HGNC Symbol; Acc: HGNC . . . |
| RCAN1 | 1827 | RCAN1 | regulator of calcineurin 1 [Source: HGNC . . . |
| REL | 5966 | REI | “REL proto-oncogene, NF-KB subunit [Sour . . . |
| RELA | 5970 | RELA | “RELA proto-oncogene, NF-KB subunit [Sou . . . |
| RELB | 5971 | RELB | “RELB proto-oncogene, NF-KB subunit [Sou . . . |
| RHOB | 388 | RHOB | ras homolog family member B [Source: HGNC . . . |
| RIPK2 | 8767 | RIPK2 | receptor interacting serine/threonine ki . . . |
| RNF19B | 127544 | RNF19B | ring finger protein 19B [Source: HGNC Sym . . . |
| SAT1 | 6303 | SAT1 | spermidine/spermine N1-acetyltransferase . . . |
| SDC4 | 6385 | SDC4 | syndecan 4 [Source: HGNC Symbol; Acc: HGNC: . . . |
| SERPINB2 | 5055 | SERPINB2 | serpin family B member 2 [Source: HGNC Sy . . . |
| SERPINB8 | 5271 | SERPINB8 | serpin family B member 8 [Source: HGNC Sy . . . |
| SERPINE1 | 5054 | SERPINE1 | serpin family E member 1 [Source: HGNC Sy . . . |
| SGK1 | 6446 | SGK1 | serum/glucocorticoid regulated kinase 1 . . . |
| SIK1 | 150094 | SIK1 | salt inducible kinase 1 [Source: HGNC Sym . . . |
| SLC16A6 | 9120 | SLC16A6 | solute carrier family 16 member 6 [Sourc . . . |
| SLC2A3 | 6515 | SLC2A3 | solute carrier family 2 member 3 [Source . . . |
| SLC2A6 | 11182 | SLC2A6 | solute carrier family 2 member 6 [Source . . . |
| SMAD3 | 4088 | SMAD3 | SMAD family member 3 [Source: HGNC Symbol . . . |
| SNN | 8303 | SNN | stannin [Source: HGNC Symbol; Acc: HGNC: 11149] |
| SOCS3 | 9021 | SOCS3 | suppressor of cytokine signaling 3 [Sour . . . |
| SOD2 | 6648 | SOD2 | superoxide dismutase 2 [Source: HGNC Symb . . . |
| SPHK1 | 8877 | SPHK1 | sphingosine kinase 1 [Source: HGNC Symbol . . . |
| SPSB1 | 80176 | SPSB1 | splA/ryanodine receptor domain and SOCS . . . |
| SQSTM1 | 8878 | SQSTM1 | sequestosome 1 [Source: HGNC Symbol; Acc: H . . . |
| STAT5A | 6776 | STAT5A | signal transducer and activator of trans . . . |
| TANK | 10010 | TANK | TRAF family member associated NFKB activ . . . |
| TAP1 | 6890 | TAP1 | “transporter 1, ATP binding cassette sub . . . |
| TGIF1 | 7050 | TGIF1 | TGFB induced factor homeobox 1 [Source: H . . . |
| TIPARP | 25976 | TIPARP | TCDD inducible poly(ADP-ribose) polymera . . . |
| TLR2 | 7097 | TLR2 | toll like receptor 2 [Source: HGNC Symbol . . . |
| TNC | 3371 | TNC | tenascin C [Source: HGNC Symbol; Acc: HGNC: . . . |
| TNF | 7124 | TNF | tumor necrosis factor [Source: HGNC Symbo . . . |
| TNFAIP2 | 7127 | TNFAIP2 | TNF alpha induced protein 2 [Source: HGNC . . . |
| TNFAIP3 | 7128 | TNFAIP3 | TNF alpha induced protein 3 [Source: HGNC . . . |
| TNFAIP6 | 7130 | TNFAIP6 | TNF alpha induced protein 6 [Source: HGNC . . . |
| TNFAIP8 | 25816 | TNFAIP8 | TNF alpha induced protein 8 [Source: HGNC . . . |
| TNFRSF9 | 3604 | TNFRSF9 | TNF receptor superfamily member 9 [Sourc . . . |
| TNFSF9 | 8744 | TNFSF9 | TNF superfamily member 9 [Source: HGNC Sy . . . |
| TNIP1 | 10318 | TNIP1 | TNFAIP3 interacting protein 1 [Source: HG . . . |
| TNIP2 | 79155 | TNIP2 | TNFAIP3 interacting protein 2 [Source: HG . . . |
| TRAF1 | 7185 | TRAF1 | TNF receptor associated factor 1 [Source . . . |
| TRIB1 | 10221 | TRIB1 | tribbles pseudokinase 1 [Source: HGNC Sym . . . |
| TRIP10 | 9322 | TRIP10 | thyroid hormone receptor interactor 10 [ . . . |
| TSC22D1 | 8848 | TSC22D1 | TSC22 domain family member 1 [Source: HGN . . . |
| TUBB2A | 7280 | TUBB2A | tubulin beta 2A class IIa [Source: HGNC S . . . |
| VEGFA | 7422 | VEGFA | vascular endothelial growth factor A [So . . . |
| YRDC | 79693 | YRDC | yrdC N6-threonylcarbamoyltransferase dom . . . |
| ZBTB10 | 65986 | ZBTB10 | zinc finger and BTB domain containing 10 . . . |
| ZC3H12A | 80149 | ZC3H12A | zinc finger CCCH-type containing 12A [So . . . |
| ZFP36 | 7538 | ZFP36 | ZFP36 ring finger protein [Source: HGNC S . . . |

In some examples the germline variant is one or more variants of any one or more of the genes listed in table 7.

TABLE 7
Genes of M5891 (HALLMARK_HYPOXIA)

| Original | NCBI | Gene | Gene |
|---|---|---|---|
| Member | GENE ID | Symbol | Description |
| ADM | 133 | ADM | adrenomedullin [Source: HGNC |
| | | | Symbol; Acc: H . . . |
| ADORA2B | 136 | ADORA2B | adenosine A2b receptor [Source: |
| | | | HGNC Symb . . . |
| AK4 | 205 | AK4 | adenylate kinase 4 [Source: |
| | | | HGNC Symbol; A . . . |
| AKAP12 | 9590 | AKAP12 | A-kinase anchoring protein 12 |
| | | | [Source: HG . . . |
| ALDOA | 226 | ALDOA | “aldolase, fructose-bisphosphate |
| | | | A [Sour . . . |
| ALDOB | 229 | ALDOB | “aldolase, fructose-bisphosphate |
| | | | B [Sour . . . |
| ALDOC | 230 | ALDOC | “aldolase, fructose-bisphosphate |
| | | | C [Sour . . . |
| AMPD3 | 272 | AMPD3 | adenosine monophosphate |
| | | | deaminase 3 [Sou . . . |
| ANGPTL4 | 51129 | ANGPTL4 | angiopoietin like 4 |
| | | | [Source: HGNC Symbol; . . . |
| ANKZF1 | 55139 | ANKZF1 | ankyrin repeat and zinc |
| | | | finger peptidyl . . . |
| ANXA2 | 302 | ANXA2 | annexin A2 [Source: HGNC |
| | | | Symbol; Acc: HGNC: . . . |
| ATF3 | 467 | ATF3 | activating transcription |
| | | | factor 3 [Sourc . . . |
| ATP7A | 538 | ATP7A | ATPase copper transporting |
| | | | alpha [Source . . . |
| B3GALT6 | 126792 | B3GALT6 | “beta-1,3-galactosyltransferase |
| | | | 6 [Sourc . . . |
| B4GALNT2 | 124872 | B4GALNT2 | “beta-1,4-N-acetyl- |
| | | | galactosaminyltransfe . . . |
| BCAN | 63827 | BCAN | brevican [Source: HGNC |
| | | | Symbol; Acc: HGNC: 23 . . . |
| BCL2 | 596 | BCL2 | BCL2 apoptosis regulator |
| | | | [Source: HGNC Sy . . . |
| BGN | 633 | BGN | biglycan [Source: HGNC |
| | | | Symbol; Acc: HGNC: 1044] |
| BHLHE40 | 8553 | BHLHE40 | basic helix-loop-helix |
| | | | family member e40 . . . |
| BNIP3L | 665 | BNIP3L | BCL2 interacting protein |
| | | | 3 like [Source: . . . |
| BRS3 | 680 | BRS3 | bombesin receptor subtype |
| | | | 3 [Source: HGNC . . . |
| BTG1 | 694 | BTG1 | BTG anti-proliferation |
| | | | factor 1 [Source: . . . |
| CA12 | 771 | CA12 | carbonic anhydrase 12 |
| | | | [Source: HGNC Symbo . . . |
| CASP6 | 839 | CASP6 | caspase 6 [Source: HGNC |
| | | | Symbol; Acc: HGNC: 1 . . . |
| CAV1 | 857 | CAV1 | caveolin 1 [Source: HGNC |
| | | | Symbol; Acc: HGNC: . . . |
| CCNG2 | 901 | CCNG2 | cyclin G2 [Source: HGNC |
| | | | Symbol; Acc: HGNC: 1 . . . |
| CCRN4L | 25819 | NOCT | nocturnin [Source: HGNC |
| | | | Symbol; Acc: HGNC: 1 . . . |
| CDKN1A | 1026 | CDKN1A | cyclin dependent kinase |
| | | | inhibitor 1A [So . . . |
| CDKN1B | 1027 | CDKN1B | cyclin dependent kinase |
| | | | inhibitor 1B [So . . . |
| CDKN1C | 1028 | CDKN1C | cyclin dependent kinase |
| | | | inhibitor 1C [So . . . |
| CHST2 | 9435 | CHST2 | carbohydrate sulfotransferase |
| | | | 2 [Source: . . . |
| CHST3 | 9469 | CHST3 | carbohydrate sulfotransferase |
| | | | 3 [Source: . . . |
| CITED2 | 10370 | CITED2 | Cbp/p300 interacting transactivator |
| | | | with . . . |
| COL5A1 | 1289 | COL5A1 | collagen type V alpha 1 chain |
| | | | [Source: HG . . . |
| CP | 1356 | CP | ceruloplasmin [Source: HGNC |
| | | | Symbol; Acc: HG . . . |
| CSRP2 | 1466 | CSRP2 | cysteine and glycine rich |
| | | | protein 2 [Sou . . . |
| CTGF | 1490 | CCN2 | cellular communication network |
| | | | factor 2 . . . |
| CXCR4 | 7852 | CXCR4 | C-X-C motif chemokine receptor |
| | | | 4 [Source . . . |
| CXCR7 | 57007 | ACKR3 | atypical chemokine receptor |
| | | | 3 [Source: HG . . . |
| CYR61 | 3491 | CCN1 | cellular communication network |
| | | | factor 1 . . . |
| DCN | 1634 | DCN | decorin [Source: HGNC |
| | | | Symbol; Acc: HGNC: 2705] |
| DDIT3 | 1649 | DDIT3 | DNA damage inducible transcript |
| | | | 3 [Sourc . . . |
| DDIT4 | 54541 | DDIT4 | DNA damage inducible transcript |
| | | | 4 [Sourc . . . |
| DPYSL4 | 10570 | DPYSL4 | dihydropyrimidinase like 4 |
| | | | [Source: HGNC . . . |
| DTNA | 1837 | DTNA | dystrobrevin alpha [Source: HGNC |
| | | | Symbol; A . . . |
| DUSP1 | 1843 | DUSP1 | dual specificity phosphatase |
| | | | 1 [Source: H . . . |
| EDN2 | 1907 | EDN2 | endothelin 2 [Source: HGNC |
| | | | Symbol; Acc: HGN . . . |
| EFNA1 | 1942 | EFNA1 | ephrin A1 [Source: HGNC |
| | | | Symbol; Acc: HGNC: 3 . . . |
| EFNA3 | 1944 | EFNA3 | ephrin A3 [Source: HGNC |
| | | | Symbol; Acc: HGNC: 3 . . . |
| EGFR | 1956 | EGFR | epidermal growth factor receptor |
| | | | [Source . . . |
| ENO1 | 2023 | ENO1 | enolase 1 [Source: HGNC |
| | | | Symbol; Acc: HGNC: 3 . . . |
| ENO2 | 2026 | ENO2 | enolase 2 [Source: HGNC |
| | | | Symbol; Acc: HGNC: 3 . . . |
| ENO3 | 2027 | ENO3 | enolase 3 [Source: HGNC |
| | | | Symbol; Acc: HGNC: 3 . . . |
| ERO1L | 30001 | ERO1A | endoplasmic reticulum |
| | | | oxidoreductase 1 a . . . |
| ERRFI1 | 54206 | ERRFI1 | ERBB receptor feedback |
| | | | inhibitor 1 [Sour . . . |
| ETS1 | 2113 | ETS1 | “ETS proto-oncogene 1, |
| | | | transcription fac . . . |
| EXT1 | 2131 | EXT1 | exostosin glycosyltransferase |
| | | | 1 [Source: . . . |
| F3 | 2152 | F3 | “coagulation factor III, |
| | | | tissue factor [. . . |
| FAM162A | 26355 | FAM162A | family with sequence similarity |
| | | | 162 memb . . . |
| FBP1 | 2203 | FBP1 | fructose-bisphosphatase 1 |
| | | | [Source: HGNC S . . . |
| FOS | 2353 | FOS | “Fos proto-oncogene, AP-1 |
| | | | transcription . . . |
| FOSL2 | 2355 | FOSL2 | “FOS like 2, AP-1 transcription |
| | | | factor s . . . |
| FOXO3 | 2309 | FOXO3 | forkhead box 03 [Source: HGNC |
| | | | Symbol; Acc: . . . |
| GAA | 2548 | GAA | alpha glucosidase [Source: HGNC |
| | | | Symbol; Ac . . . |
| GALK1 | 2584 | GALK1 | galactokinase 1 [Source: HGNC |
| | | | Symbol; Acc: . . . |
| GAPDH | 2597 | GAPDH | glyceraldehyde-3-phosphate |
| | | | dehydrogenase . . . |
| GAPDHS | 26330 | GAPDHS | “glyceraldehyde-3-phosphate |
| | | | dehydrogenas . . . |
| GBE1 | 2632 | GBE1 | “1,4-alpha-glucan branching |
| | | | enzyme 1 [So . . . |
| GCK | 2645 | GCK | glucokinase [Source: HGNC |
| | | | Symbol; Acc: HGNC . . . |
| GCNT2 | 2651 | GCNT2 | glucosaminyl (N-acetyl) |
| | | | transferase 2 (I . . . |
| GLRX | 2745 | GLRX | glutaredoxin [Source: HGNC |
| | | | Symbol; Acc: HGN . . . |
| GPC1 | 2817 | GPC1 | glypican 1 [Source: HGNC |
| | | | Symbol; Acc: HGNC: . . . |
| GPC3 | 2719 | GPC3 | glypican 3 [Source: HGNC |
| | | | Symbol; Acc: HGNC: . . . |
| GPC4 | 2239 | GPC4 | glypican 4 [Source: HGNC |
| | | | Symbol; Acc: HGNC: . . . |
| GPI | 2821 | GPI | glucose-6-phosphate isomerase |
| | | | [Source: HG . . . |
| GRHPR | 9380 | GRHPR | glyoxylate and hydroxypyruvate |
| | | | reductase . . . |
| GYS1 | 2997 | GYS1 | glycogen synthase 1 |
| | | | [Source: HGNC Symbol; . . . |
| HAS1 | 3036 | HAS1 | hyaluronan synthase 1 |
| | | | [Source: HGNC Symbo . . . |
| HDLBP | 3069 | HDLBP | high density lipoprotein |
| | | | binding protein . . . |
| HEXA | 3073 | HEXA | hexosaminidase subunit alpha |
| | | | [Source: HGN . . . |
| HK1 | 3098 | HK1 | hexokinase 1 [Source: HGNC |
| | | | Symbol; Acc: HGN . . . |
| HK2 | 3099 | HK2 | hexokinase 2 [Source: HGNC |
| | | | Symbol; Acc: HGN . . . |
| HMOX1 | 3162 | HMOX1 | heme oxygenase 1 [Source: HGNC |
| | | | Symbol; Acc . . . |
| HOXB9 | 3219 | HOXB9 | homeobox B9 [Source: HGNC |
| | | | Symbol; Acc: HGNC . . . |
| HS3ST1 | 9957 | HS3ST1 | heparan sulfate-glucosamine |
| | | | 3-sulfotrans . . . |
| HSPA5 | 3309 | HSPA5 | heat shock protein family |
| | | | A (Hsp70) memb . . . |
| IDS | 3423 | IDS | iduronate 2-sulfatase |
| | | | [Source: HGNC Symbo . . . |
| IER3 | 8870 | IER3 | immediate early response |
| | | | 3 [Source: HGNC . . . |
| IGFBP1 | 3484 | IGFBP1 | insulin like growth factor |
| | | | binding prote . . . |
| IGFBP3 | 3486 | IGFBP3 | insulin like growth factor |
| | | | binding prote . . . |
| IL6 | 3569 | IL6 | interleukin 6 [Source: HGNC |
| | | | Symbol; Acc: HG . . . |
| ILVBL | 10994 | ILVBL | ilvB acetolactate synthase |
| | | | like [Source: . . . |
| INHA | 3623 | INHA | inhibin subunit alpha |
| | | | [Source: HGNC Symbo . . . |
| IRS2 | 8660 | IRS2 | insulin receptor substrate |
| | | | 2 [Source: HGN . . . |
| ISG20 | 3669 | ISG20 | interferon stimulated |
| | | | exonuclease gene 2 . . . |
| JMJD6 | 23210 | JMJD6 | “jumonji domain containing |
| | | | 6, arginine d . . . |
| JUN | 3725 | JUN | “Jun proto-oncogene, AP-1 |
| | | | transcription . . . |
| KDELR3 | 11015 | KDELR3 | KDEL endoplasmic reticulum |
| | | | protein reten . . . |
| KDM3A | 55818 | KDM3A | lysine demethylase 3A |
| | | | [Source: HGNC Symbo . . . |
| KIF5A | 3798 | KIF5A | kinesin family member |
| | | | 5A [Source: HGNC Sy . . . |
| KLF6 | 1316 | KLF6 | Kruppel like factor 6 |
| | | | [Source: HGNC Symbo . . . |
| KLF7 | 8609 | KLF7 | Kruppel like factor 7 |
| | | | [Source: HGNC Symbo . . . |
| KLHL24 | 54800 | KLHL24 | kelch like family member |
| | | | 24 [Source: HGNC . . . |
| LALBA | 3906 | LALBA | lactalbumin alpha |
| | | | [Source: HGNC Symbol; Ac . . . |
| LARGE | 9215 | LARGE1 | LARGE xylosyl- and |
| | | | glucuronyltransferase . . . |
| LDHA | 3939 | LDHA | lactate dehydrogenase A |
| | | | [Source: HGNC Sym . . . |
| LDHC | 3948 | LDHC | lactate dehydrogenase C |
| | | | [Source: HGNC Sym . . . |
| LOX | 4015 | LOX | lysyl oxidase [Source: HGNC |
| | | | Symbol; Acc: HG . . . |
| LXN | 56925 | LXN | latexin [Source: HGNC |
| | | | Symbol; Acc: HGNC: 13347] |
| MAFF | 23764 | MAFF | MAF bZIP transcription |
| | | | factor F [Source: . . . |
| MAP3K1 | 4214 | MAP3K1 | mitogen-activated protein |
| | | | kinase kinase . . . |
| MIF | 4282 | MIF | macrophage migration |
| | | | inhibitory factor [. . . |
| MT1E | 4493 | MT1E | metallothionein 1E |
| | | | [Source: HGNC Symbol; A . . . |
| MT2A | 4502 | MT2A | metallothionein 2A |
| | | | [Source: HGNC Symbol; A . . . |
| MX11 | 4601 | MX11 | “MAX interactor 1, |
| | | | dimerization protein . . . |
| MYH9 | 4627 | MYH9 | myosin heavy chain 9 |
| | | | [Source: HGNC Symbol . . . |
| NAGK | 55577 | NAGK | N-acetylglucosamine kinase |
| | | | [Source: HGNC . . . |
| NCAN | 1463 | NCAN | neurocan [Source: HGNC |
| | | | Symbol; Acc: HGNC: 2465] |
| NDRG1 | 10397 | NDRG1 | N-myc downstream regulated 1 |
| | | | [Source: HGN . . . |
| NDST1 | 3340 | NDST1 | N-deacetylase and N- |
| | | | sulfotransferase 1 [. . . |
| NDST2 | 8509 | NDST2 | N-deacetylase and N- |
| | | | sulfotransferase 2 [. . . |
| NEDD4L | 23327 | NEDD4L | NEDD4 like E3 ubiquitin |
| | | | protein ligase [. . . |
| NFIL3 | 4783 | NFIL3 | “nuclear factor, interleukin |
| | | | 3 regulated . . . |
| NR3C1 | 2908 | NR3C1 | nuclear receptor subfamily |
| | | | 3 group C mem . . . |
| P4HA1 | 5033 | P4HA1 | prolyl 4-hydroxylase subunit |
| | | | alpha 1 [So . . . |
| P4HA2 | 8974 | P4HA2 | prolyl 4-hydroxylase subunit |
| | | | alpha 2 [So . . . |
| PAM | 5066 | PAM | peptidylglycine alpha-amidating |
| | | | monooxyg . . . |
| PCK1 | 5105 | PCK1 | phosphoenolpyruvate carboxykinase |
| | | | 1 [Sou . . . |
| PDGFB | 5155 | PDGFB | platelet derived growth factor |
| | | | subunit B . . . |
| PDK1 | 5163 | PDK1 | pyruvate dehydrogenase kinase |
| | | | 1 [Source: . . . |
| PDK3 | 5165 | PDK3 | pyruvate dehydrogenase kinase |
| | | | 3 [Source: . . . |
| PFKFB3 | 5209 | PFKFB3 | “6-phosphofructo-2-kinase/fructose- |
| | | | 2,6-b . . . |
| PFKL | 5211 | PFKL | “phosphofructokinase, liver type |
| | | | [Source . . . |
| PFKP | 5214 | PFKP | “phosphofructokinase, platelet |
| | | | [Source: H . . . |
| PGAM2 | 5224 | PGAM2 | phosphoglycerate mutase 2 |
| | | | [Source: HGNC S . . . |
| PGF | 5228 | PGF | placental growth factor |
| | | | [Source: HGNC Sym . . . |
| PGK1 | 5230 | PGK1 | phosphoglycerate kinase 1 |
| | | | [Source: HGNCS . . . |
| PGM1 | 5236 | PGM1 | phosphoglucomutase 1 |
| | | | [Source: HGNC Symbol . . . |
| PGM2 | 55276 | PGM2 | phosphoglucomutase 2 |
| | | | [Source: HGNC Symbol . . . |
| PHKG1 | 5260 | PHKG1 | phosphorylase kinase catalytic |
| | | | subunit g . . . |
| PIM1 | 5292 | PIM1 | “Pim-1 proto-oncogene, |
| | | | serine/threonine . . . |
| PKLR | 5313 | PKLR | pyruvate kinase L/R |
| | | | [Source: HGNC Symbol; . . . |
| PKP1 | 5317 | PKP1 | plakophilin 1 [Source: HGNC |
| | | | Symbol; Acc: HG . . . |
| PLAC8 | 51316 | PLAC8 | placenta associated 8 |
| | | | [Source: HGNC Symbo . . . |
| PLAUR | 5329 | PLAUR | “plasminogen activator, |
| | | | urokinase recept . . . |
| PLIN2 | 123 | PLIN2 | perilipin 2 [Source: HGNC |
| | | | Symbol; Acc: HGNC . . . |
| PNRC1 | 10957 | PNRC1 | proline rich nuclear receptor |
| | | | coactivato . . . |
| PPARGC1A | 10891 | PPARGC1A | PPARG coactivator 1 alpha |
| | | | [Source: HGNC S . . . |
| PPFIA4 | 8497 | PPFIA4 | PTPRF interacting protein |
| | | | alpha 4 [Sourc . . . |
| PPP1R15A | 23645 | PPP1R15A | protein phosphatase 1 |
| | | | regulatory subunit . . . |
| PPP1R3C | 5507 | PPP1R3C | protein phosphatase 1 |
| | | | regulatory subunit . . . |
| PRDX5 | 25824 | PRDX5 | peroxiredoxin 5 [Source: |
| | | | HGNC Symbol; Acc: . . . |
| PRKCA | 5578 | PRKCA | protein kinase C alpha |
| | | | [Source: HGNC Symb . . . |
| PRKCDBP | 112464 | CAVIN3 | caveolae associated protein |
| | | | 3 [Source: HG . . . |
| PTRF | 284119 | CAVIN1 | caveolae associated protein |
| | | | 1 [Source: HG . . . |
| PYGM | 5837 | PYGM | “glycogen phosphorylase, |
| | | | muscle associat . . . |
| RBPJ | 3516 | RBPJ | recombination signal binding |
| | | | protein for . . . |
| RORA | 6095 | RORA | RAR related orphan receptor A |
| | | | [Source: HG . . . |
| RRAGD | 58528 | RRAGD | Ras related GTP binding D |
| | | | [Source: HGNC S . . . |
| S100A4 | 6275 | S100A4 | S100 calcium binding protein A4 |
| | | | [Source: . . . |
| SAP30 | 8819 | SAP30 | Sin3A associated protein 30 |
| | | | [Source: HGNC . . . |
| SCARB1 | 949 | SCARB1 | scavenger receptor class B |
| | | | member 1 [Sou . . . |
| SDC2 | 6383 | SDC2 | syndecan 2 [Source: HGNC |
| | | | Symbol; Acc: HGNC: . . . |
| SDC3 | 9672 | SDC3 | syndecan 3 [Source: HGNC |
| | | | Symbol; Acc: HGNC: . . . |
| SDC4 | 6385 | SDC4 | syndecan 4 [Source: HGNC |
| | | | Symbol; Acc: HGNC: . . . |
| SELENBP1 | 8991 | SELENBP1 | selenium binding protein 1 |
| | | | [Source: HGNC . . . |
| SERPINE1 | 5054 | SERPINE1 | serpin family E member 1 |
| | | | [Source: HGNC Sy . . . |
| SIAH2 | 6478 | SIAH2 | siah E3 ubiquitin protein |
| | | | ligase 2 [Sour . . . |
| SLC25A1 | 6576 | SLC25A1 | solute carrier family 25 |
| | | | member 1 [Sourc . . . |
| SLC2A1 | 6513 | SLC2A1 | solute carrier family 2 |
| | | | member 1 [Source . . . |
| SLC2A3 | 6515 | SLC2A3 | solute carrier family 2 |
| | | | member 3 [Source . . . |
| SLC2A5 | 6518 | SLC2A5 | solute carrier family 2 |
| | | | member 5 [Source . . . |
| SLC37A4 | 2542 | SLC37A4 | solute carrier family 37 |
| | | | member 4 [Sourc . . . |
| SLC6A6 | 6533 | SLC6A6 | solute carrier family 6 |
| | | | member 6 [Source . . . |
| SRPX | 8406 | SRPX | sushi repeat containing |
| | | | protein X-linked . . . |
| STBD1 | 8987 | STBD1 | starch binding domain 1 |
| | | | [Source: HGNC Sym . . . |
| STC1 | 6781 | STC1 | stanniocalcin 1 [Source: HGNC |
| | | | Symbol; Acc: . . . |
| STC2 | 8614 | STC2 | stanniocalcin 2 [Source: HGNC |
| | | | Symbol; Acc: . . . |
| SULT2B1 | 6820 | SULT2B1 | sulfotransferase family |
| | | | 2B member 1 [Sou . . . |
| TES | 26136 | TES | testin LIM domain protein |
| | | | [Source: HGNC S . . . |
| TGFB3 | 7043 | TGFB3 | transforming growth factor |
| | | | beta 3 [Sourc . . . |
| TGFBI | 7045 | TGFBI | transforming growth factor |
| | | | beta induced . . . |
| TGM2 | 7052 | TGM2 | transglutaminase 2 [Source: |
| | | | HGNC Symbol; A . . . |
| TIPARP | 25976 | TIPARP | TCDD inducible poly(ADP-ribose) |
| | | | polymera . . . |
| TKTL1 | 8277 | TKTL1 | transketolase like 1 [Source: |
| | | | HGNC Symbol . . . |
| TMEM45A | 55076 | TMEM45A | transmembrane protein 45A |
| | | | [Source: HGNC S . . . |
| TNFAIP3 | 7128 | TNFAIP3 | TNF alpha induced protein 3 |
| | | | [Source: HGNC . . . |
| TPBG | 7162 | TPBG | trophoblast glycoprotein |
| | | | [Source: HGNC Sy . . . |
| TPD52 | 7163 | TPD52 | tumor protein D52 [Source: |
| | | | HGNC Symbol; Ac . . . |
| TPI1 | 7167 | TPI1 | triosephosphate isomerase 1 |
| | | | [Source: HGNC . . . |
| TPST2 | 8459 | TPST2 | tyrosylprotein sulfotransferase |
| | | | 2 [Sourc . . . |
| UGP2 | 7360 | UGP2 | UDP-glucose pyrophosphorylase |
| | | | 2 [Source: . . . |
| VEGFA | 7422 | VEGFA | vascular endothelial growth |
| | | | factor A [So ... |
| VHL | 7428 | VHL | von Hippel-Lindau tumor |
| | | | suppressor [Sour . . . |
| VLDLR | 7436 | VLDLR | very low density lipoprotein |
| | | | receptor [S . . . |
| WISP2 | 8839 | CCN5 | cellular communication |
| | | | network factor 5 . . . |
| WSB1 | 26118 | WSB1 | WD repeat and SOCS box |
| | | | containing 1 [Sou . . . |
| XPNPEP1 | 7511 | XPNPEP1 | X-prolyl aminopeptidase |
| | | | 1 [Source: HGNC S . . . |
| ZFP36 | 7538 | ZFP36 | ZFP36 ring finger protein |
| | | | [Source: HGNC S . . . |
| ZNF292 | 23036 | ZNF292 | zinc finger protein 292 |
| | | | [Source: HGNC Sym . . . |

In some examples, a subject may have at least one germline variant in a plurality of gene sets. For example, a subject may have a germline variant in at least one gene from two of the gene sets selected from: M5891 (HALLMARK_HYPOXIA); M5932 (HALLMARK_INFLAMMATORY_RESPONSE); M5953 (HALLMARK_KRAS_SIGNALING_UP); M5957 (HALLMARK_PANCREAS_BETA_CELLS); M5923 (HALLMARK_PI3K_AKT_MTOR_SIGNALING); and/or M5890 (HALLMARK_TNFA_SIGNALING_VIA_NFKB).

For example, a subject may have a germline variant in at least one gene from three of the gene sets selected from: M5891 (HALLMARK_HYPOXIA); M5932 (HALLMARK_INFLAMMATORY_RESPONSE); M5953 (HALLMARK_KRAS_SIGNALING_UP); M5957 (HALLMARK_PANCREAS_BETA_CELLS); M5923 (HALLMARK_PI3K_AKT_MTOR_SIGNALING); and/or M5890 (HALLMARK_TNFA_SIGNALING_VIA_NFKB).

In some examples, a subject may have at least one germline variant in a plurality of gene sets. For example, a subject may have a germline variant in at least one gene from two of the gene sets selected from: M5932 (HALLMARK_INFLAMMATORY_RESPONSE); M5953 (HALLMARK_KRAS_SIGNALING_UP); M5957 (HALLMARK_PANCREAS_BETA_CELLS); M5923 (HALLMARK_PI3K_AKT_MTOR_SIGNALING); and/or M5890 (HALLMARK_TNFA_SIGNALING_VIA_NFKB).

For example, a subject may have a germline variant in at least one gene from three of the gene sets selected from: M5932 (HALLMARK_INFLAMMATORY_RESPONSE); M5953 (HALLMARK_KRAS_SIGNALING_UP); M5957 (HALLMARK_PANCREAS_BETA_CELLS); M5923 (HALLMARK_PI3K_AKT_MTOR_SIGNALING); and/or M5890 (HALLMARK_TNFA_SIGNALING_VIA_NFKB).

In some examples a subject may have a germline variant in at least one gene selected from the genes of Table 1 and Table 2. In some examples a subject may have a germline variant in at least one selected from the genes of Table 1 and Table 3. In some examples a subject may have a germline variant in at least one selected from the genes of Table 1 and Table 4. In some examples a subject may have a germline variant in at least one selected from the genes of Table 1 and Table 5. In some examples a subject may have a germline variant in at least one selected from the genes of Table 1 and Table 6. In some examples a subject may have a germline variant in at least one selected from the genes of Table 1 and Table 7.

In some examples a subject may have a germline variant in at least one selected from the genes of Table 2 and Table 3. In some examples a subject may have a germline variant in at least one selected from the genes of Table 2 and Table 4. In some examples a subject may have a germline variant in at least one selected from the genes of Table 2 and Table 5. In some examples a subject may have a germline variant in at least one selected from the genes of Table 2 and Table 6. In some examples a subject may have a germline variant in at least one selected from the genes of Table 2 and Table 7.

In some examples a subject may have a germline variant in at least one selected from the genes of Table 3 and Table 4. In some examples a subject may have a germline variant in at least one selected from the genes of Table 3 and Table 5. In some examples a subject may have a germline variant in at least one selected from the genes of Table 3 and Table 6. In some examples a subject may have a germline variant in at least one selected from the genes of Table 3 and Table 7.

In some examples a subject may have a germline variant in at least one selected from the genes of Table 4 and Table 5. In some examples a subject may have a germline variant in at least one selected from the genes of Table 4 and Table 6. In some examples a subject may have a germline variant in at least one selected from the genes of Table 4 and Table 7.

In some examples a subject may have a germline variant in at least one selected from the genes of Table 5 and Table 6. In some examples a subject may have a germline variant in at least one selected from the genes of Table 5 and Table 7.

In some examples a subject may have one or more germline variants in at least one gene of the genes up-regulated by activation of the PI3K/AKT/mTOR pathway; genes defining inflammatory response; genes up-regulated by KRAS activation; genes up-regulated in response to low oxygen levels; and genes regulated by NF-kB in response to tumour necrosis factor (TNF). For example a subject may have one more germline variants of one or more genes from Table 2, Table 3, Table 4, Table 6 and Table 7. For example a subject may have one more germline variants of one or more genes from M5932 (HALLMARK_INFLAMMATORY_RESPONSE); M5891 (HALLMARK_HYPOXIA); M5953 (HALLMARK_KRAS_SIGNALING_UP); M5923 (HALLMARK_PI3K_AKT_MTOR_SIGNALING); and M5890 (HALLMARK_TNFA_SIGNALING_VIA_NFKB). In some examples, the subject may have a germline variant in at least 4 genes (e.g. PIKFYVE, MYD88, CAB39, and RPS6KA1) from M5923 (HALLMARK_PI3K_AKT_MTOR_SIGNALING); at least 5 genes (e.g., IRAK2, IL2RB, MSR1, ITGB8, and PIK3R5) from M5932 (HALLMARK_INFLAMMATORY_RESPONSE); at least 3 genes (e.g. MMP10, HKDC1, and RBM4) from M5953 (HALLMARK_KRAS_SIGNALING_UP); at least 6 genes (e.g. GAPDHS, GRHPR, PGM1, SELENBP1, NAGK, and SLC6A6) from M5891 (HALLMARK_HYPOXIA); and 4 genes (e.g. DDX58, KYNU, NR4A1, and DENND5A) from M5890 (HALLMARK_TNFA_SIGNALING_VIA_NFKB).

In some examples, the germline variant may be a variant of at least one of: PIKFYVE, MYD88, CAB39, RPS6KA1, IRAK2, IL2RB, MSR1, ITGB8, PIK3R5, MMP10, HKDC1, RBM4, GAPDHS, GRHPR, PGM1, SELENBP1, NAGK, SLC6A6, DDX58, KYNU, NR4A1, and/or DENND5A.

In some examples a subject may have one or more germline variants in at least one gene of the genes up-regulated by activation of the PI3K/AKT/mTOR pathway; genes defining inflammatory response; and genes up-regulated by KRAS activation. For example a subject may have one more germline variants of one or more genes from Table 2, Table 3, and Table 4. For example a subject may have one more germline variants of one or more genes from M5932 (HALLMARK_INFLAMMATORY_RESPONSE); M5953 (HALLMARK_KRAS_SIGNALING_UP); and M5923 (HALLMARK_PI3K_AKT_MTOR_SIGNALING. In some examples, the subject may have a germline variant in at least 4 genes (e.g. PIKFYVE, MYD88, CAB39, and RPS6KA1) from M5923 (HALLMARK_PI3K_AKT_MTOR_SIGNALING); at least 5 genes (e.g., IRAK2, IL2RB, MSR1, ITGB8, and PIK3R5) from M5932 (HALLMARK_INFLAMMATORY_RESPONSE); and at least 3 genes (e.g. MMP10, HKDC1, and RBM4) from M5953 (HALLMARK_KRAS_SIGNALING_UP.

In some examples, the germline variant may be a variant of at least one of: PIKFYVE, MYD88, CAB39, RPS6KA1, IRAK2, IL2RB, MSR1, ITGB8, PIK3R5, MMP10, HKDC1 and/or RBM4.

PIKFYVE encodes an enzyme (PIKfyve; also known as phosphatidylinositol-3-phosphate 5-kinase type III or PIPKIII) that phosphorylates the D-5 position in PtdIns and phosphatidylinositol-3-phosphate (PtdIns3P) to make PtdIns5P and PtdIns (3,5) biphosphate. PIKfyve preferentially phosphorylates D-3 phosphorylated PtdIns. In addition to being a lipid kinase, PIKfyve also has protein kinase activity. PIKfyve regulates endomembrane homeostasis and plays a role in the biogenesis of endosome carrier vesicles from early endosomes. The protein plays a key role in cell entry of Ebola virus and SARS-CoV-2 by endocytosis Mutations in this gene cause corneal fleck dystrophy (CFD); an autosomal dominant disorder characterized by numerous small white flecks present in all layers of the corneal stroma. Histologically, these flecks appear to be keratocytes distended with lipid and mucopolysaccharide filled intracytoplasmic vacuoles.

MYD88 encodes a cytosolic adapter protein that plays a central role in the innate and adaptive immune response. The protein functions as an essential signal transducer in the interleukin-1 and Toll-like receptor signalling pathways. These pathways regulate the activation of numerous proinflammatory genes. The encoded protein consists of an N-terminal death domain and a C-terminal Toll-interleukin1 receptor domain. Patients with defects in this gene have an increased susceptibility to pyogenic bacterial infections.

CAB39 encodes Calcium Binding Protein 39. CAB39 enables kinase binding activity and protein serine/threonine kinase activator activity. It is involved in intracellular signal transduction; peptidyl-serine phosphorylation; and positive regulation of protein phosphorylation. It is located in the extracellular exosome. It has been implicated in hepatocellular carcinoma and is used as a biomarker of hepatocellular carcinoma and pancreatic cancer.

RPS6KA1 encodes a member of the RSK (ribosomal S6 kinase) family of serine/threonine kinases. This kinase contains 2 nonidentical kinase catalytic domains and phosphorylates various substrates, including members of the mitogen-activated kinase (MAPK) signalling pathway. The activity of this protein has been implicated in controlling cell growth and differentiation.

IRAK2 encodes the interleukin-1 receptor-associated kinase 2, one of two putative serine/threonine kinases that become associated with the interleukin-1 receptor (IL1R) upon stimulation. IRAK2 is reported to participate in the IL1-induced upregulation of NF-kappaB.

IL2RB encodes the interleukin 2 receptor, which is involved in T cell-mediated immune responses, and is present in 3 forms with respect to ability to bind interleukin 2. The low affinity form is a monomer of the alpha subunit and is not involved in signal transduction. The intermediate affinity form consists of an alpha/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from interleukin 2. The protein encoded by this gene represents the beta subunit and is a type I membrane protein. The use of alternative promoters results in multiple transcript variants encoding the same protein. The protein is primarily expressed in the hematopoietic system. The use by some variants of an alternate promoter in an upstream long terminal repeat (LTR) results in placenta-specific expression.

MSR1 encodes the class A macrophage scavenger receptors, which include three different types (1, 2, 3) generated by alternative splicing of this gene. These receptors or isoforms are macrophage-specific trimeric integral membrane glycoproteins and have been implicated in many macrophage-associated physiological and pathological processes including atherosclerosis, Alzheimer's disease, and host defense. The isoforms type 1 and type 2 are functional receptors and are able to mediate the endocytosis of modified low density lipoproteins (LDLs). The isoform type 3 does not internalize modified LDL (acetyl-LDL) despite having the domain shown to mediate this function in the types 1 and 2 isoforms. It has an altered intracellular processing and is trapped within the endoplasmic reticulum, making it unable to perform endocytosis. The isoform type 3 can inhibit the function of isoforms type 1 and type 2 when co-expressed, indicating a dominant negative effect and suggesting a mechanism for regulation of scavenger receptor activity in macrophages.

ITGB8 encodes a member of the integrin beta chain family and encodes a single-pass type I membrane protein with a VWFA domain and four cysteine-rich repeats. This protein noncovalently binds to an alpha subunit to form a heterodimeric integrin complex. In general, integrin complexes mediate cell-cell and cell-extracellular matrix interactions and this complex plays a role in human airway epithelial proliferation. Alternatively spliced variants which encode different protein isoforms have been described; however, not all variants have been fully characterized.

PIK3R5 encodes the 101 kD regulatory subunit of the class I PI3K gamma complex, which is a dimeric enzyme, consisting of a 110 kD catalytic subunit gamma and a regulatory subunit of either 55, 87 or 101 kD. This protein recruits the catalytic subunit from the cytosol to the plasma membrane through high-affinity interaction with G-beta-gamma proteins. Phosphatidylinositol 3-kinases (PI3Ks) phosphorylate the inositol ring of phosphatidylinositol at the 3-prime position, and play important roles in cell growth, proliferation, differentiation, motility, survival and intracellular trafficking. The PI3Ks are divided into three classes: I, II and III, and only the class I PI3Ks are involved in oncogenesis.

MMP10 encodes a member of the peptidase M10 family of matrix metalloproteinases (MMPs). Proteins in this family are involved in the breakdown of extracellular matrix in normal physiological processes, such as embryonic development, reproduction, and tissue remodelling, as well as in disease processes, such as arthritis and metastasis. The encoded preproprotein is proteolytically processed to generate the mature protease. This secreted protease breaks down fibronectin, laminin, elastin, proteoglycan core protein, gelatins, and several types of collagen. The gene is part of a cluster of MMP genes on chromosome 11.

HKDC1 encodes a member of the hexokinase protein family. The encoded protein is involved in glucose metabolism, and reduced expression may be associated with gestational diabetes mellitus. High expression of this gene may also be associated with poor prognosis in hepatocarcinoma.

RBM4 encodes RNA Binding Motif Protein 4. This is an RNA-binding factor involved in multiple aspects of cellular processes like alternative splicing of pre-mRNA and translation regulation. It modulates alternative 5′-splice site and exon selection; acts as a muscle cell differentiation-promoting factor; activates exon skipping of the PTB pre-mRNA during muscle cell differentiation; antagonizes the activity of the splicing factor PTBP1 to modulate muscle cell-specific exon selection of alpha tropomyosin; and binds to intronic pyrimidine-rich sequence of the TPM1 and MAPT pre-mRNAs. It is required for the translational activation of PER1 mRNA in response to circadian clock. It binds directly to the 3′-UTR of the PER1 mRNA and exerts a suppressive activity on Cap-dependent translation via binding to CU-rich responsive elements within the 3′UTR of mRNAs, a process increased under stress conditions or during myocytes differentiation. It also recruits EIF4A1 to stimulate IRES-dependent translation initiation in response to cellular stress and associates to internal ribosome entry segment (IRES) in target mRNA species under stress conditions. It also plays a role for miRNA-guided RNA cleavage and translation suppression by promoting association of AGO2-containing miRNPs with their cognate target mRNAs.

GAPDHS encodes a protein belonging to the glyceraldehyde-3-phosphate dehydrogenase family of enzymes that play an important role in carbohydrate metabolism. Like its somatic cell counterpart, this sperm-specific enzyme functions in a nicotinamide adenine dinucleotide-dependent manner to remove hydrogen and add phosphate to glyceraldehyde 3-phosphate to form 1,3-diphosphoglycerate. During spermiogenesis, this enzyme may play an important role in regulating the switch between different energy-producing pathways, and it is required for sperm motility and male fertility.

GRHPR encodes Glyoxylate And Hydroxypyruvate Reductase, an enzyme with hydroxypyruvate reductase, glyoxylate reductase, and D-glycerate dehydrogenase enzymatic activities. The enzyme has widespread tissue expression and has a role in metabolism.

PGM1 encodes an isozyme of phosphoglucomutase (PGM) and belongs to the phosphohexose mutase family. There are several PGM isozymes, which are encoded by different genes and catalyze the transfer of phosphate between the 1 and 6 positions of glucose. In most cell types, this PGM isozyme is predominant, representing about 90% of total PGM activity. In red cells, PGM2 is a major isozyme. This gene is highly polymorphic. Mutations in this gene cause glycogen storage disease type 14.

SELENBP1 encodes a member of the selenium-binding protein family. Selenium is an essential nutrient that exhibits potent anticarcinogenic properties, and deficiency of selenium may cause certain neurologic diseases. The effects of selenium in preventing cancer and neurologic diseases may be mediated by selenium-binding proteins, and decreased expression of this gene may be associated with several types of cancer. The encoded protein may play a selenium-dependent role in ubiquitination/deubiquitination-mediated protein degradation.

NAGK encodes a member of the N-acetylhexosamine kinase family. The encoded protein catalyzes the conversion of N-acetyl-D-glucosamine to N-acetyl-D-glucosamine 6-phosphate, and is the major mammalian enzyme which recovers amino sugars.

SLC6A6 encodes a multi-pass membrane protein that is a member of a family of sodium and chloride-ion dependent transporters. The encoded protein transports taurine and beta-alanine. There is a pseudogene for this gene on chromosome 21.

DDX58 encodes a protein containing RNA helicase-DEAD box protein motifs and a caspase recruitment domain (CARD). It is involved in viral double-stranded (ds) RNA recognition and the regulation of the antiviral innate immune response. Mutations in this gene are associated with Singleton-Merten syndrome 2.

KYNU encodes kynureninase. Kynureninase is a pyridoxal-5′-phosphate (pyridoxal-P) dependent enzyme that catalyzes the cleavage of L-kynurenine and L-3-hydroxykynurenine into anthranilic and 3-hydroxyanthranilic acids, respectively. Kynureninase is involved in the biosynthesis of NAD cofactors from tryptophan through the kynurenine pathway.

NR4A1 encodes Nuclear Receptor Subfamily 4 Group A Member 1, a member of the steroid-thyroid hormone-retinoid receptor superfamily. Expression is induced by phytohemagglutinin in human lymphocytes and by serum stimulation of arrested fibroblasts. The encoded protein acts as a nuclear transcription factor. Translocation of the protein from the nucleus to mitochondria induces apoptosis.

DENND5A encodes DENN Domain Containing 5A, a DENN-domain-containing protein that functions as a RAB-activating guanine nucleotide exchange factor (GEF). This protein catalyzes the conversion of GDP to GTP and thereby converts inactive GDP-bound Rab proteins into their active GTP-bound form. The encoded protein is recruited by RAB6 onto Golgi membranes and is therefore referred to as RAB6-interacting protein 1. This protein binds with RAB39 as well.

In some examples, the presence of a variant may be at least one germline variant of at least one gene from one or more of the gene sets selected from: M5932 (HALLMARK_INFLAMMATORY_RESPONSE); M5953 (HALLMARK_KRAS_SIGNALING_UP); and/or M5923 (HALLMARK_PI3K_AKT_MTOR_SIGNALING).

In some examples, the at least one germline variant may be predictive of time to biochemical relapse. Variants in any one of M5932 (HALLMARK_INFLAMMATORY_RESPONSE); M5953 (HALLMARK_KRAS_SIGNALING_UP); and/or M5923 (HALLMARK_PI3K_AKT_MTOR_SIGNALING) may in particular be related to time to biochemical recurrence.

In some examples, the subject may have high-grade prostate cancer (e.g. a high-grade tumour) and may have at least one germline variant of at least one gene from at one or more of the gene sets selected from: M5953 (HALLMARK_KRAS_SIGNALING_UP); M5957 (HALLMARK_PANCREAS_BETA_CELLS); M5923 (HALLMARK_PI3K_AKT_MTOR_SIGNALING); and/or M5890 (HALLMARK_TNFA_SIGNALING_VIA_NFKB). These gene sets may be indicative of time to BCR and/or likelihood of BCR particularly in subjects with high-grade tumours.

In some examples a subject may have one or more germline variants in at least one gene of the genes up-regulated by activation of the PI3K/AKT/mTOR pathway; genes up-regulated by KRAS activation; genes up-regulated in response to low oxygen levels; and genes regulated by NF-kB in response to tumour necrosis factor (TNF). For example a subject may have one more germline variants of one or more genes from Table 2, Table 4, Table 6 and Table 7. For example a subject may have one more germline variants of one or more genes from M5891 (HALLMARK_HYPOXIA); M5953 (HALLMARK_KRAS_SIGNALING_UP); M5923 (HALLMARK_PI3K_AKT_MTOR_SIGNALING); and M5890 (HALLMARK_TNFA_SIGNALING_VIA_NFKB). In some examples, the subject may have a germline variant in at least 4 genes (e.g. PIKFYVE, MYD88, CAB39, and RPS6KA1) from M5923 (HALLMARK_PI3K_AKT_MTOR_SIGNALING); at least 3 genes (e.g. MMP10, HKDC1, and RBM4) from M5953 (HALLMARK_KRAS_SIGNALING_UP); at least 6 genes (e.g. GAPDHS, GRHPR, PGM1, SELENBP1, NAGK, and SLC6A6) from M5891 (HALLMARK_HYPOXIA); and 4 genes (e.g. DDX58, KYNU, NR4A1, and DENND5A) from M5890 (HALLMARK_TNFA_SIGNALING_VIA_NFKB)

In some examples, the subject has high-grade prostate cancer and the at least one germline variant includes a germline variant of at least one of GAPDHS, GRHPR, PGM1, SELENBP1, NAGK, SLC6A6, PIKFYVE, MYD88, CAB39, RPS6KA1, DDX58, KYNU, NR4A1, DENND5A, MMP10, HKDC1, and/or RBM4.

### Subject

The term “subject” as used herein refers to, for example, humans, chimpanzees, Rhesus monkeys, dogs, cows, horses, cats, mice, rats, chickens, zebrafish, fruit flies, mosquitoes, C. elegans and frogs provided that they also have a prostate. The subject is preferably a mammal, such as a human. The subject is most commonly male.

The subject may be referred to herein as a patient. The terms “subject”, “individual”, and “patient” are used herein interchangeably. The subject can be symptomatic (e.g., the subject presents symptoms associated with prostate cancer), or the subject can be asymptomatic (e.g., the subject does not present symptoms associated with prostate cancer).

The subject may be diagnosed with, be at risk of developing or present with symptoms of prostate cancer. The subject may have, or be suspected of having (e.g. present with symptoms or a history indicative or suggestive of), prostate cancer.

Accordingly, in some examples, the subject has prostate cancer. In some examples, the subject has early stage prostate cancer. An example of an early stage of disease is when the subject has the initial symptoms of prostate cancer but has not yet developed sufficient symptoms for diagnosis of disease. In such examples, the method may be considered as a method for determining the risk of relapse if the subject does develop prostate cancer. In some examples, the subject does not have prostate cancer.

As used herein, an individual that “does not have prostate cancer” is an individual that has histologically normal-appearing prostate tissue. Methods for histologically testing prostate tissue and identifying whether an individual has histologically normal-appearing prostate tissue are well known in the art, see for example Litwin M S and Tan H J., The Diagnosis and Treatment of Prostate Cancer: A Review. JAMA. 2017 Jun. 27; 317(24):2532-254. A control sample that is obtained from an individual that does not have prostate cancer in this context therefore refers to a biological fluid sample (e.g. a blood or urine sample, as appropriate) that has been obtained from an individual of the same species, where the individual has histologically normal-appearing prostate tissue. Examples of individuals that do not have prostate cancer include individuals with benign prostate hyperplasia, prostatitis and/or an enlarged prostate.

In particular examples, the subject has localised prostate cancer. In other examples, the subject has metastatic prostate cancer.

The terms “cancer” and “cancerous” refer to or describe the physiological condition that is typically characterized by unregulated cell growth. Examples of cancer include cancer of the urogenital tract, such as prostate cancer. As used herein, the term “prostate cancer” refers to all stages and all forms of cancer arising from the tissue of the prostate gland.

Methods of diagnosing and staging prostate cancer are well known in the art. For example, according to the tumor, node, metastasis (TNM) staging system of the American Joint Committee on Cancer (AJCC), AJCC Cancer Staging Manual (7th Ed., 2010), the various stages of prostate cancer are defined as follows: Tumor: T1: clinically inapparent tumor not palpable or visible by imaging, T1a: tumor incidental histological finding in 5% or less of tissue resected, T1b: tumor incidental histological finding in more than 5% of tissue resected, T1c: tumor identified by needle biopsy; T2: tumor confined within prostate, T2a: tumor involves one half of one lobe or less, T2b: tumor involves more than half of one lobe, but not both lobes, T2c: tumor involves both lobes; T3: tumor extends through the prostatic capsule, T3a: extracapsular extension (unilateral or bilateral), T3b: tumor invades seminal vesicle(s); T4: tumor is fixed or invades adjacent structures other than seminal vesicles (bladder neck, external sphincter, rectum, levator muscles, or pelvic wall). Node: NO: no regional lymph node metastasis; N1: metastasis in regional lymph nodes. Metastasis: MO: no distant metastasis; M1: distant metastasis present.

The Gleason Grading system is also commonly used to help evaluate the prognosis of men with prostate cancer. Together with other parameters, it is incorporated into a strategy of prostate cancer staging, which predicts prognosis and helps guide therapy. A Gleason “score” or “grade” is given to prostate cancer based upon its microscopic appearance. Tumors with a low Gleason score typically grow slowly enough that they may not pose a significant threat to the patients in their lifetimes. These patients are monitored (“watchful waiting” or “active surveillance”) over time. Cancers with a higher Gleason score are more aggressive and have a worse prognosis, and these patients are generally treated with surgery (e.g., radical prostatectomy) and, in some cases, therapy (e.g., radiation, hormone, ultrasound, chemotherapy, immunotherapy). Gleason scores (or sums) comprise grades of the two most common tumor patterns. These patterns are referred to as Gleason patterns 1-5, with pattern 1 being the most well-differentiated. Most have a mixture of patterns. To obtain a Gleason score or grade, the dominant pattern is added to the second most prevalent pattern to obtain a number between 2 and 10. The Gleason Grades include: G1: well differentiated (slight anaplasia) (Gleason 2-4); G2: moderately differentiated (moderate anaplasia) (Gleason 5-6); G3-4: poorly differentiated/undifferentiated (marked anaplasia) (Gleason 7-10).

In some examples, the subject may have a high-grade prostate cancer. High-grade prostate cancer refers to a subject having a prostate cancer with a Gleason grade of 3-4. In some examples, a Gleason grade of 3-5. In some examples, high-grade prostate cancer refers to a subject having a prostate cancer with a Gleason score of 4+3 or higher. In some examples, high-grade prostate cancer refers to a subject having a prostate cancer with a Gleason score of 4+3 or higher and/or a Gleason grade of 3-5.

The methods described herein may be used to identify subjects that have an increased risk of relapse if they do develop prostate cancer. In this context, the phrase “increased risk” indicates that the subject has a higher level of risk (or likelihood) that they will experience a particular clinical outcome. A subject may be classified (stratified) into a risk group or classified at a level of risk based on the methods described herein, e.g. high, medium, or low risk. A “risk group” is a group of subjects or individuals with a similar level of risk for a particular clinical outcome.

In some examples, the subject suffers from or has previously suffered from prostate cancer and undergone radical therapy. Radical therapy refers to vigorous treatment that aims at the complete cure of a disease rather than the mere relief of symptoms. This is in comparison to conservative treatment or therapy. Radical therapies in the case of prostate cancer may include surgery, radiation therapy, cryotherapy, hormone therapy, and/or chemotherapy.

In particular, the subject may have previously undergone radical surgery such as a radical proctectomy and/or radical radiotherapy. Radical prostatectomy refers to removal of the entire prostate gland, the seminal vesicles and the vas deferens.

In some examples the methods described herein are carried out before a radical prostatectomy is conducted while in some examples the methods are carried out after a radical prostatectomy is conducted.

### Treatments

The methods described herein can further comprise selecting, and optionally administering, a treatment regimen for the subject based on the prognosis or stratification (i.e., based on the presence of the variations as described herein). Treatment can include, for example, surgery (e.g., radical proctectomy) and, in some cases, therapy (e.g., radiation, hormone, ultrasound, chemotherapy, immunotherapy), or combinations thereof. However, in some cases, immediate treatment may not be required, and the subject may be selected for active surveillance. The selection of a treatment or further treatment can be based on the detection of one or more of the germline variants described herein. For example, the treatment may be selected depending on whether a subject is stratified as having an increased likelihood of BCR and/or a reduced time to BCR.

For example, when one or more of the germline variants as described herein are detected a subject who does not have prostate cancer, has prostate cancer but not undergone treatment, is suspected of having prostate cancer but has not undergone treatment and/or is at risk of developing prostate cancer a radical therapy may be administered as an initial therapy. In some examples, the radical therapy may include a radical proctectomy and/or radical radiotherapy. In some examples, the radical therapy may be administered early to a subject having one or more of the germline variants as described herein than a subject who does not have one or more of the germline variants as described herein. In some examples, more radical therapy may be administered to a subject having one or more of the germline variants as described herein than a subject who does not have one or more of the germline variants as described herein.

In some examples, when one or more of the germline variants as described herein are detected in a subject who does not have prostate cancer, has prostate cancer but not undergone treatment, is suspected of having prostate cancer but has not undergone treatment and/or is at risk of developing prostate cancer active surveillance is initiated or increased.

In some examples, wherein when one or more of the germline variants as described herein are not detected in a subject who has prostate cancer but not undergone treatment, is suspected of having prostate cancer but has not undergone treatment and/or is at risk of developing prostate cancer an alternative to radical therapy may be administered. For example, any suitable therapy other than a radical therapy may be administered.

In some examples, when one or more of the germline variants as described herein are detected in a subject who has or has had prostate cancer and undergone radical therapy the subject may be administered a further therapy. For example, a further radical therapy. It will be understood, that if a subject has undergone a radical prostatectomy that the further treatment may be any radical therapy other than a further radical proctectomy. For example, wherein the subject has undergone radical radiotherapy the subject may undergo a radical proctectomy. For example, wherein the subject has undergone radical proctectomy the subject may undergo a radical radiotherapy.

In some examples, the further therapy may be administered early to a subject having one or more of the germline variants as described herein than a subject who does not have one or more of the germline variants as described herein. In some examples, more further therapy may be administered a subject having one or more of the germline variants as described herein than a subject who does not have one or more of the germline variants as described herein.

In some examples, when one or more of the germline variants as described herein are detected in a subject who has or has had prostate cancer and undergone radical therapy active surveillance is initiated or increased.

As such, there is provided herein a method of determining a treatment regimen for a prostate cancer patient based up the detection of one or more germline variants as described herein.

As used herein, the terms “active surveillance”, “monitoring” and “watchful waiting” are used interchangeably herein to mean closely monitoring a patient's condition without giving any treatment until symptoms appear or change. For example, in prostate cancer, watchful waiting is usually used in older men with other medical problems and early-stage disease.

As used herein, the terms “treat”, “treating” and “treatment” are taken to include an intervention performed with the intention of preventing the development or altering the pathology of a condition, disorder or symptom (i.e. in this case prostate cancer). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the symptoms of prostate cancer, for example of at least 5%, 10%, 20%, 30%, 40%, 50%, 60%, 70%, 80%, 90% or 100% when compared to the symptoms before treatment. In the context of prostate cancer, appropriate treatment may include surgery and/or therapy.

As used herein, the term “surgery” applies to surgical methods undertaken for removal of cancerous tissue, including pelvic lymphadenectomy, radical prostatectomy, transurethral resection of the prostate (TURP), excision, dissection, and tumor biopsy/removal.

As used herein, the term “therapy” includes radiation, hormonal therapy, cryosurgery, chemotherapy, immunotherapy, biologic therapy, and high-intensity focused ultrasound.

The type of treatment will vary depending on the particular form of prostate cancer that the subject has, is suspected of having, is at risk of developing, or is suspected of being at risk of developing.

For example, if the subject has, is suspected of having, is at risk of having, or is suspected of being at risk of having, metastatic prostate cancer, the subject may benefit from treatment with for example androgen deprivation therapy, radiotherapy, and/or immunotherapy. Accordingly, the method may include the step of administering one or more of these treatments to the subject. Other suitable treatments are well known to a person of skill in the art and depend on the specific symptoms of the subject. Prostate cancer treatments include prostatectomy, radiotherapy, hormonal therapy (e.g., using GnRH antagonists, GnRH agonists, antiandrogens), chemotherapy, and high intensity focused ultrasound. For example, when a subject is identified herein as having (i.e. diagnosed with) high grade and/or metastatic prostate cancer and/or castrate resistant prostate cancer and has an increased likelihood of BCR and/or reduced time to BCR determined by the methods described herein, they may be treated with a treatment selected from the group consisting of: (i) hormone therapy (e.g. LHRH agonists/GnRH antagonists/Tablets such as Goserelin (Zoladex®), Leuprorelin acetate (Prostap® or Lutrate®), Triptorelin (Decapeptyl® or Gonapeptyl Depot®), Buserelin acetate (Suprefact®), Histrelin (Vantas®), Degarelix (Firmagon®), Bicalutamide (Casodex®), Cyproterone acetate (Cyprostat®), Flutamide (Drogenil®), Abiraterone acetate (Zytiga®), or Nilutamide (Nilandron®))
(ii) Chemotherapy (e.g. Docetaxel (Taxotere®), Cabazitaxel (Jevtana®), Strontium-89 (Metastron®), Samarium-153 (Quadramet®), Enzalutamide (Xtandi®), Radium-223 dichloride (Xofigo®), or Apalutamide (Erleada®))
(iii) Steroids (e.g. Prednisolone, Dexamethasone, Hydrocortisone);
(iv) Sipuleucel-T (Provenge®) (to treat advanced, recurrent prostate cancer), or Ketoconazole, optionally in combination with a treatment selected from the group consisting of: radical prostatectomy, external beam radiotherapy/Brachytherapy (with or without hormone therapy), High Intensity Focused Ultrasound (HIFU), Cryotherapy and Trans-urethral resection of the prostate (TURP); and
(v) Monoclonal antibody therapies (e.g. Pembrolizumab (keytruda), Avastin (bevacizumab), Erbitux (cetuximab), Rituxan (rituximab) and Herceptin (trastuzumab)).

It will be understood that if the subject has already undergone a radical prostatectomy, the treatment may any treatment other than radical proctectomy.

Androgens are also closely linked to prostate cancer treatment, with androgen deprivation therapy (ADT) being the principal pharmacological strategy for locally advanced and metastatic disease. ADT utilises drugs to inhibit gonadal and extra-gonadal androgen biosynthesis and competitive AR antagonists to block androgen binding and abrogate AR function. Accordingly, if the subject has, is suspected of having, is at risk of having, or is suspected of being at risk of having, metastatic prostate cancer, a preferred method may include the step of administering androgen deprivation therapy to the subject.

As a further example, if the subject has, is suspected of having, is at risk of having, or is suspected of being at risk of having, non-metastatic, localised, prostate cancer, the subject may benefit from active surveillance or surgery. Accordingly, the method may include the step of administering one or more of these treatments to the subject. Other suitable treatments are well known to a person of skill in the art and depend on the specific symptoms of the subject. For example, when a subject is identified herein as having (i.e. diagnosed with) low grade prostate cancer and has an increased likelihood of BCR and/or reduced time to BCR determined by the methods described herein, they may be placed under active surveillance or be treated with a treatment selected from the group consisting of: radical prostatectomy, external beam radiotherapy/Brachytherapy (with or without hormone therapy), High Intensity Focused Ultrasound (HIFU), Cryotherapy and Trans-urethral resection of the prostate (TURP).

When a therapeutic agent or other treatment is administered, it is administered in an amount and/or for a duration that is effective to treat the prostate cancer or to reduce the likelihood (or risk) of prostate cancer developing in the future. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain analytes that are indicative of prostate cancer. It should be understood that the therapeutic agents described herein are used to treat and/or prevent prostate cancer. Thus, in some cases, they may be used prophylactically in subjects at risk of developing prostate cancer or who are at risk of relapse of prostate cancer. Thus, in some cases, an effective amount is that amount which can lower the risk of, slow or perhaps prevent altogether the development of prostate cancer. It will be recognized when the therapeutic agent is used in acute circumstances, it is used to prevent one or more medically undesirable results that typically flow from such adverse events. Methods for selecting a suitable treatment, an appropriate dose thereof and modes of administration will be apparent to one of ordinary skill in the art.

The medications or treatments described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration. The medications may also be given in e.g. tablet form or in solution. Several appropriate medications and means for administration of the same are well known for treatment of prostate cancer.

### Biomarker Panel

Also provided herein is a signature biomarker panel that may be used for determining the prognosis of a subject. For example, the panel may be characteristic of a subject's likelihood of BCR and/or of time to BCR. A biomarker panel refers to more than one biomarker (i.e. germline variant described herein) that can be detected from a subject sample that together, are associated with prognosis of the subject. The presence of the biomarkers may not be individually quantified as an absolute value, but the measured values may be normalized and the normalized value is aggregated (e.g., summed or weighted and summed, etc.) for inclusion within a biomarker composite score.

The signature biomarker panel may include all or a fragment of one or more of the genes found in Table 1. The polynucleotides can be attached to a substrate, such as a glass slide or microarray chip. In some examples, detection of at least one germline variant may be by detecting hybridization (or a lack thereof) of fragments of a subject's genetic material corresponding to each gene in the panel.

The signature biomarker panel may include at least one germline variant s described herein. In some examples, the signature panel may include all of the germline variants described herein. In particular the signature panel that includes a germline variant of at least one of PIKFYVE, MYD88, CAB39, RPS6KA1, IRAK2, IL2RB, MSR1, ITGB8, PIK3R5, MMP10, HKDC1, RBM4, GAPDHS, GRHPR, PGM1, SELENBP1, NAGK, SLC6A6, DDX58, KYNU, NR4A1, and/or DENND5A.

In particular, the signature biomarker panel may include at least one germline variant of at least one of PIKFYVE, MYD88, CAB39, RPS6KA1, IRAK2, IL2RB, MSR1, ITGB8, PIK3R5, MMP10, HKDC1 and/or RBM4.

In some examples, the patient or subject suffers from high-grade prostate cancer and the signature panel includes at least one germline variant of at least one of: GAPDHS, GRHPR, PGM1, SELENBP1, NAGK, SLC6A6, PIKFYVE, MYD88, CAB39, RPS6KA1, DDX58, KYNU, NR4A1, DENND5A, MMP10, HKDC1, and/or RBM4.

The germline variants may be detected in a sample from a subject using any known methods in the art, for example using immunodetection, PCR (realtime PCR, RT-PCR, qPCR, TaqMan PCR).

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Abstract

Background: Germline variants explain more than a third of prostate cancer (PrCa) risk, but very few associations have been identified between heritable factors and clinical progression.

Objective: To find rare germline variants that predict time to biochemical recurrence (BCR) after radical treatment in men with PrCa, and understand the genetic factors associated with such progression.

Design, Setting and Participants: Whole-genome sequencing data from blood DNA were analysed for 850 PrCa patients with radical treatment from the Pan Prostate Cancer Group (PPCG consortium) from UK, Canada, Germany, Australia and France. Findings were validated using 383 patients from The Cancer Genome Atlas (TCGA).

Outcome Measurements and Statistical analysis: 15,822 rare (MAF<1%) predicted-deleterious coding germline mutations were identified. Optimal multifactor and univariate Cox regression models were built to predict time to BCR after radical treatment, using germline variants grouped by functionally annotated gene-sets. Models were tested for robustness using bootstrap resampling.

Results: optimal Cox regression multifactor models showed that rare predicted-deleterious germline variants in “Hallmark” gene-sets were consistently associated with altered time to BCR. Three gene-sets had a statistically significant association with risk-elevated outcome when modelling all samples: PI3K/AKT/mTOR, Inflammatory response and KRAS signalling (up). PI3K/AKT/mTOR and KRAS signalling (up) were also associated among patients with higher grade cancer, as were Pancreas-beta cells, TNFA signalling via NKFB and Hypoxia, the latter of which was validated in the independent TCGA dataset.

### Materials and Methods

Sequencing of DNA from PrCa Patients

Whole-genome sequencing (WGS) data derived from whole blood samples were collated for PrCa patients from member countries of the Pan Prostate Cancer Group (PPCG, http://panprostate.orq; Australia n=133, Canada n=288, France n=15, Germany n=230, UK n=184; Table 8, further characteristics in Table 9). The study presented here combines data from patients following RP, and a small subset of samples with radical radiotherapy (RT; 8%) from the Canadian study group. Samples are collectively referred to as having radica00001 treatment.

Samples were collected according to criteria outlined in the method below. Collection was subject to the International Cancer Genome Consortium (ICGC) standards of ethical consent. Collection and analysis of the Australian samples received institutional review board approval (Epworth Health 34506; Melbourne Health 2019.058). WGS was performed using Illumina technology to ≥30× depth.

TABLE 8
Number of samples, genes and variants contributed, by study, also showing the number of samples
with high-Gleason score (>3 + 4; Gleason grade group 3-5), the numbers of samples in
each set with biochemical recurrence (BCR), numbers associated with mutations that are predicted-
deleterious, and how many of those are known deleterious/loss-of-function (LoF) mutations.

| | | | | | Number of |
|---|---|---|---|---|---|
| | | | | Number of | predicted- |
| | | Samples | Samples | genes with | deleterious |
| | European | used in | with high- | predicted- | mutations |
| | Genome- | study after | Gleason | deleterious | included in |
| | phenome | QC (with | score (with | mutations | analysis |
| Study | Archive ID | BCR) | BCR) | (LoF) | (LoF) |
| Melbourne, | EGAD00001004182 | 133 (79) | 110 (70) | 2,917 (1,884) | 3,728 (2,473) |
| Australian | | | | | |
| research | | | | | |
| group | | | | | |
| Canadian | EGAD00001004170 | 288 (92) | 63 (22) | 4,579 (2,637) | 5,900 (3,154) |
| Prostate | | | | | |
| Cancer | | | | | |
| Genome | | | | | |
| Network | | | | | |
| French | EGAD00001003835 | 15 (10) | 15 (10) | 409 (255) | 393 (243) |
| ICGC | | | | | |
| Prostate | | | | | |
| Cancer | | | | | |
| Group | | | | | |
| Germany | EGAD00001005997 | 230 (68) | 85 (45) | 3,787 (2,160) | 4,761 (2,404) |
| ICGC | | | | | |
| Prostate | | | | | |
| Cancer | | | | | |
| Group - | | | | | |
| Early Onset | | | | | |
| CRUK-ICGC | EGAC00001000852 | 184 (36) | 63 (22) | 3,365 (2,073) | 4,071 (2,401) |
| Prostate | | | | | |
| Group, UK | | | | | |
| Total | | 850 (285) | 336 (169) | 8,455 (5,792) | 15,822 (9,006) |

TABLE 9
Patient characteristics distributed by study, with
The Cancer Genome Atlas (TCGA) set appended.

| Study | Clinical variable | Class | # patients |
|---|---|---|---|
| Australia | Relapsed | 0 | 54 |
| | | 1 | 79 |
| | Gleason Score | <4 + 3 | 23 |
| | | ≥4 + 3 | 110 |
| | PSA | median | 8.90 |
| | Pathological T | T stage < 3 | 37 |
| | | T stage ≥ 3 | 96 |
| | Age at diagnosis | median | 66 |
| | | <65 | 61 |
| | | ≥65 | 72 |
| | Follow-up (days) | median | 259 |
| Canada | Relapsed | 0 | 196 |
| | | 1 | 92 |
| | Gleason Score | <4 + 3 | 225 |
| | | ≥4 + 3 | 63 |
| | PSA | median | 6.90 |
| | Pathological T | T stage < 3 | 187 |
| | | T stage ≥ 3 | 101 |
| | Age at diagnosis | median | 64 |
| | | <65 | 151 |
| | | ≥65 | 137 |
| | Follow-up (days) | median | 229 |
| France | Relapsed | 0 | 5 |
| | | 1 | 10 |
| | Gleason Score | <4 + 3 | 0 |
| | | ≥4 + 3 | 15 |
| | PSA | median | 7.90 |
| | Pathological T | T stage < 3 | 1 |
| | | T stage ≥ 3 | 14 |
| | Age at diagnosis | median | 64 |
| | | <65 | 8 |
| | | ≥65 | 7 |
| | Follow-up (days) | median | 210 |
| Germany | Relapsed | 0 | 162 |
| | | 1 | 68 |
| | Gleason Score | <4 + 3 | 145 |
| | | ≥4 + 3 | 85 |
| | PSA | median | 9.20 |
| | Pathological T | T stage < 3 | 136 |
| | | T stage ≥ 3 | 94 |
| | Age at diagnosis | median | 48 |
| | | <65 | 204 |
| | | ≥65 | 256 |
| | Follow-up (days) | median | 753 |
| UK | Relapsed | 0 | 148 |
| | | 1 | 36 |
| | Gleason Score | <4 + 3 | 121 |
| | | ≥4 + 3 | 63 |
| | PSA | median | 8.00 |
| | Pathological T | T stage < 3 | 61 |
| | | T stage ≥ 3 | 123 |
| | Age at diagnosis | median | 62 |
| | | <65 | 113 |
| | | ≥65 | 71 |
| | Follow-up (days) | median | 991 |
| TCGA | Relapsed | 0 | 339 |
| | | 1 | 44 |
| | Gleason Score | <4 + 3 | 150 |
| | | ≥4 + 3 | 233 |
| | Pathological T | T stage < 3 | 142 |
| | | T stage ≥ 3 | 241 |
| | Follow-up (days) | median | 417 |

Burrows-Wheeler Aligner (BWA, [6]) was used to align sequencing data to the GRCh37 human genome (human_g1k_v37) with PCR duplicates removed [7]. Sequencing data have been deposited at the European Genome-phenome Archive (https://eqa-archive.org, study IDs in Table 8) and is available upon request.

### Sample Collection and Criteria

Unless otherwise stated, all patients underwent radical prostatectomy (RadP), and biochemical recurrence (BCR) was defined as two consecutive post-RadP PSA measurements of more than 0.2 ng/ml (backdated to the date of the first increase). If a patient had successful salvage radiation therapy, this was not considered BCR. If PSA continued to rise after radiation therapy, BCR was backdated to first PSA>0.2 ng/ml. If a patient received other salvage treatment (such as hormones or chemotherapy), this was considered BCR.

### Melbourne, Australian Research Group

All patients were hormone-naïve at the time of treatment. Patients were retrospectively selected from our tissue biorepositry enriching for patients with high grade disease.

DNA and RNA were simultaneously extracted using the Allprep Micro Kit (Qiagen, CA) following manufacturer instructions and including on column DNAse digestion of the RNA. Genomic DNA was extracted from fresh frozen samples of whole blood with the DNeasy Blood & Tissue Kit (Qiagen, Maryland) following manufacturer instructions.

### Canadian Prostate Cancer Genome Network

All patients underwent either image-guided radiotherapy (IGRT) or radical prostatectomy (RadP), with curative intent, for pathologically confirmed prostate cancer. All patients were hormone-naïve at the time of definitive local therapy. In the IGRT cohort, a single ultrasound-guided needle biopsy was obtained before the start of therapy. Fresh-frozen RadP specimens were obtained from the University Health Network (UHN) Pathology BioBank or from the Genito-Urinary BioBank of the Centre Hospitalier Universitaire de Quebec (CHUQ).

For IGRT patients, BCR was defined as a rise in PSA concentration of more than 2.0 ng/ml above the nadir (after radiotherapy, PSA levels drop and stabilize at the nadir).

Whole blood was collected and informed consent, consistent with local Research Ethics Board (REB) and International Cancer Genome Consortium (ICGC) guidelines, was obtained at the time of clinical follow-up. All patients were NOMO as an entry criterion for the study.

Fraser M, Sabelnykova V Y, Yamaguchi T N, et al. Genomic hallmarks of localized, non-indolent prostate cancer. Nature. 2017; 541:359-64. https://doi.org/10.1038/nature20788

### French ICGC Prostate Cancer Group

The French cohort is comprised of Caucasian patients with aggressive prostate cancer characterized by a clinical-pathological aggressive pattern (D'Amico 3 with primary Gleason grade 4). All patients were treatment-naïve at the time of surgery.

They provided written informed consent, consistent with local Research Ethics Board (REB) and the International Cancer Genome Consortium (ICGC) guidelines. For germline DNA extraction, saliva was collected using the Oragene DNA collection kit (DNA Genotek Inc) at the time of consent.

### Germany ICGC Prostate Cancer Group—Early Onset (EO)

The EO cohort is composed of patients diagnosed with PC<=55 years of age. Except for two patients (PCA125 and PCA176) who received pre-operation hormone therapy with LH-RH, the patients did not receive any neo-adjuvant radiotherapy, androgen deprivation therapy, or chemotherapy prior to the surgical removal of tumour tissue.

DNA and RNA were extracted as described previously: Weischenfeldt J, Simon R, Feuerbach L, et al. Integrative genomic analyses reveal an androgen-driven somatic alteration landscape in early-onset prostate cancer. Cancer Cell. 2013; 23:159-70. https://doi.org/10.1016/j.ccr.2013.01.002

### CRUK-ICGC Prostate Group, UK

Fresh frozen tumour and matching whole blood samples were collected from radical prostatectomy patients treated at The Royal Marsden NHS Foundation Trust, London, at the Addenbrooke's Hospital, Cambridge, or at Oxford University Hospitals NHS Trust. Consequently those samples with >40% tumour content and their matching blood samples were whole genome sequenced. All patients were treatment naïve at the time of surgery.

This data was collected as part of the CRUK-ICGC prostate project within the framework of ICGC and more information can be found in previous publications: Cooper C S, Eeles R, Wedge D C, et al. Analysis of the genetic phylogeny of multifocal prostate cancer identifies multiple independent clonal expansions in neoplastic and morphologically normal prostate tissue. Nat Genet. 2015; 47:367-72. https://doi.org/10.1038/ng.3221; and Wedge D C, Gundem G, Mitchell T, et al. Sequencing of prostate cancers identifies new cancer genes, routes of progression and drug targets. Nat Genet. 2018; 50:682-92. https://doi.org/10.1038/s41588-018-0086-z.

### Variant Calling

Variant calling was performed with The Genome Analysis Toolkit pipeline (GATK v4.0) [8] following GATK best practice recommendations for germline SNV and indel calling [9, 10], apart from for the German samples which were called using FreeBayes v1.1.0 [11] and processed as described by Gerhauser et al. [12], normalised with vt v0.5 [13](Supplementary Method 3). This analysis was restricted to variants within protein-coding transcript sequences according to GENCODE v29 [14].

In brief, after read alignment and duplicate removal, Base Quality Score Recalibration (BSQR) was performed to detect errors introduced by the sequencer and correct the quality scores assigned to each base call. Variants were called using GATK HaplotypeCaller via local de-novo assembly of haplotypes in a region, producing one gvcf file per sample. Joint-genotyping was performed on the whole cohort, producing one multi-sample VCF file. Variant Quality Score Recalibration (VQSR) was performed to remove false positive variants by comparing them against a high quality set. Genotype posteriors were calculated using 1000 Genomes phase 3 VCF. Indels were left-aligned, and multi-allelic variants were decomposed into bi-allelic components.

### Quality Control, Variant Annotation and Prioritization

Low-quality variants and samples were removed based on established QC protocols [15-17]. Samples from related individuals were excluded (using R package SNPRelate method identity-by-descent [18]), or with non-European ancestry (using Principal Component Analysis relative to 2,504 samples from the 1000 Genomes Project [19]). Picard tools v2.23.8 [20] was used to remove samples with a mean insert size <250 bp, AT or GC dropout >5%, <95% aligned reads, >5% mismatch rate, <80% with ≥20× coverage or >5% missing call rate. Using verifyBam/D v1.1.2 [21] samples with >3% sample contamination were removed. Variants with a missing call rate in >5% of the samples were excluded, monomorphic loci, those in repetitive regions (simple repeats, segmental duplications and centromeric regions) and where the ExAC major allele frequency in any population was >1%. 3% of the submitted samples were excluded based on ancestry, while 2% were removed because of sequencing quality. One sample was removed due to relatedness.

Post-QC variants were annotated using the germline variant Effect Predictor (VEP v101) and loss-of-function transcript effect estimator (LOFTEE) package [22]. For downstream analyses only variants categorised as deleterious/loss-of-function were retained, comprising those with protein-truncating mutations (nonsense, frameshift and splice site variants) occurring in the first 95% of the protein, as well as predicted-deleterious missense variants with a CADD PHRED score >30 [23](Table 8).

### Pathways and Gene-Sets

For pathway level analysis, all 50 “Hallmark” gene-sets from GSEA MsigDB were considered [24 (Downloaded April 2017)], along with the BROCA extended panel of 66 genes and 175 curated DNA repair genes (DRG) [16, 17](Tables 10, 11 and 12)

TABLE 10
list of gene sets studied. The “hallmark” gene sets are available via https://www.gsea-msigdb.org/gsea/msigdb/genesets.jsp?collection=H which
provides a list of all genes included in each set and which are expressly incorporated herein.

| | | |
|---|---|---|
| HALLMARK_ADIPOGENESIS | HALLMARK_G2M_CHECKPOINT | HALLMARK_NOTCH_SIGNALING |
| HALLMARK_ALLOGRAFT_REJECTION | HALLMARK_GLYCOLYSIS | HALLMARK_OXIDATIVE_{—} |
| | | PHOSPHORYLATION |
| HALLMARK_ANDROGEN_RESPONSE | HALLMARK_HEDGEHOG_SIGNALING | HALLMARK_P53_PATHWAY |
| HALLMARK_ANGIOGENESIS | HALLMARK_HEME_METABOLISM | HALLMARK_PANCREAS_BETA_CELLS |
| HALLMARK_APICAL_JUNCTION | HALLMARK_HYPOXIA | HALLMARK_PEROXISOME |
| HALLMARK_APICAL_SURFACE | HALLMARK_IL2_STAT5_SIGNALING | HALLMARK_PI3K_AKT_MTOR_SIGNALING |
| HALLMARK_APOPTOSIS | HALLMARK_IL6_JAK_{—} | HALLMARK_PROTEIN_SECRETION |
| | STAT3_SIGNALING | |
| HALLMARK_BILE_ACID_METABOLISM | HALLMARK_INFLAMMATORY_{—} | HALLMARK_REACTIVE_OXYGEN_{—} |
| | RESPONSE | SPECIES_PATHWAY |
| HALLMARK_CHOLESTEROL_{—} | HALLMARK_INTERFERON_{—} | HALLMARK_SPERMATOGENESIS |
| HOMEOSTASIS | ALPHA_RESPONSE | |
| HALLMARK_COAGULATION | HALLMARK_INTERFERON_{—} | HALLMARK_TGF_BETA_SIGNALING |
| | GAMMA_RESPONSE | |
| HALLMARK_COMPLEMENT | HALLMARK_KRAS_SIGNALING_DN | HALLMARK_TNFA_SIGNALING_VIA_NFKB |
| HALLMARK_DNA_REPAIR | HALLMARK_KRAS_SIGNALING_UP | HALLMARK_UNFOLDED_{—} |
| | | PROTEIN_RESPONSE |
| HALLMARK_E2F_TARGETS | HALLMARK_MITOTIC_SPINDLE | HALLMARK_UV_RESPONSE_DN |
| HALLMARK_EPITHELIAL_{—} | HALLMARK_MTORC1_SIGNALING | HALLMARK_UV_RESPONSE_UP |
| MESENCHYMAL_TRANSITION | | |
| HALLMARK_ESTROGEN_{—} | HALLMARK_MYC_TARGETS_V1 | HALLMARK_WNT_BETA_{—} |
| RESPONSE_EARLY | | CATENIN_SIGNALING |
| HALLMARK_ESTROGEN_{—} | HALLMARK_MYC_TARGETS_V2 | HALLMARK_XENOBIOTIC_METABOLISM |
| RESPONSE_LATE | | |
| HALLMARK_FATTY_ACID_METABOLISM | HALLMARK_MYOGENESIS | BROCA |
| DRG | | |

TABLE 11
Genes in the BROCA extended panel gene set.

| | | | | | | | |
|---|---|---|---|---|---|---|---|
| AKT1 | MBD4 | CETN2 | POLG | BARD1 | POLD1 | ATRIP | POLI |
| APC | MDC1 | CHAF1A | TOP2A | BLM | RNF4 | POLE | POLK |
| ATM | MDM2 | CHEK1 | TOPBP1 | BRCA1 | RNF8 | POT1 | POLL |
| ATR | MDM4 | CHEK2 | TP53 | BRCA2 | RPA1 | PRKAR1A | POLM |
| AXIN2 | MGMT | CLK2 | TP53BP1 | BRIP1 | RPA2 | PRSS1 | POLN |
| BAP1 | MLH1 | CLSPN | TREX1 | C17orf70 | RPA3 | PTCH1 | POLQ |
| BARD1 | MLH3 | DCLRE1A | TREX2 | C19orf40 | RPA4 | PTEN | PRKDC |
| BMPR1A | MMS19 | DCLRE1B | UBE2A | CCNB1 | RRM2B | RAD51B | PRPF19 |
| BRCA1 | MNAT1 | DCLRE1C | UBE2B | CCND1 | SETMAR | RAD51C | PTEN |
| BRCA2 | MPG | DDB1 | UBE2I | CCNE1 | SHFM1 | RAD51D | RAD1 |
| BRIP1 | MRE11A | DDB2 | UBE2N | CCNH | SHPRH | RB1 | RAD17 |
| CDH1 | MSH2 | DMC1 | UBE2V2 | CDC25A | SLX1A | RECQL | RAD18 |
| CDK4 | MSH3 | DUT | UNG | CDC25C | SLX1B | RET | RAD23A |
| CDKN2A | MSH4 | E2F1 | USP1 | CDH1 | SLX4 | RINT1 | RAD23B |
| CHEK1 | MSH5 | EME1 | UVSSA | CDK1 | SMUG1 | RPS20 | RAD50 |
| CHEK2 | MSH6 | EME2 | WDR48 | CDK2 | SPO11 | SDHB | RAD51 |
| PIK3CA | POLE | PMS2 | WRN | PDGFRA | SPRTN | SDHC | RNF168 |

| | | | | | | | |
|---|---|---|---|---|---|---|---|
| ATR | MUS81 | APTX | GTF2H1 | ALKBH2 | GEN1 | AKT1 | PCNA |
| CTNNA1 | MUTYH | ATM | GTF2H2 | ALKBH3 | HELQ | APLF | PER1 |
| EPCAM | NABP2 | ENDOV | GTF2H3 | APEX1 | HLTF | MSH2 | PER2 |
| FAM175A | NBN | ERCC1 | GTF2H4 | APEX2 | HUS1 | MSH6 | PMS1 |
| FANCM | NEIL1 | ERCC2 | GTF2H5 | CDK4 | LIG1 | MUTYH | PMS2 |
| FH | NEIL2 | ERCC3 | H2AFX | CDK7 | LIG3 | NBN | PMS2P3 |
| FLCN | NEIL3 | ERCC4 | REV1 | CDKN1A | LIG4 | NF1 | PNKP |
| GALNT12 | NHEJ1 | ERCC5 | REV3L | CDKN1B | MAD2L2 | NTHL1 | RAD51B |
| GEN1 | NTHL1 | ERCC6 | XAB2 | FANCF | POLB | PALB2 | RAD51C |
| GREM1 | NUDT1 | ERCC8 | XPA | FANCG | RBBP8 | SDHD | RAD51D |
| HOXB13 | OGG1 | EXO1 | XPC | FANCI | RDM1 | SLX4 | RAD52 |
| MEN1 | PALB2 | FAN1 | XRCC1 | FANCL | RECQL | SMAD4 | RAD54B |
| MET | PARP1 | FANCA | XRCC2 | FANCM | RECQL4 | SMARCA4 | RAD54L |
| MITF | PARP2 | FANCB | XRCC3 | FBXO18 | STK11 | TP53 | RAD9A |
| MLH1 | PARP3 | FANCC | XRCC4 | FEN1 | TDG | VHL | RB1 |
| MRE11A | POLH | FANCD2 | XRCC5 | GADD45A | TDP1 | XRCC2 | RECQL5 |
| POLD1 | RIF1 | FANCE | XRCC6 | PALLD | TDP2 | | |

TABLE 12
Genes in the DNA repair gene (DRG) panel.

| | | | | | | |
|---|---|---|---|---|---|---|
| AKT1 | CETN2 | FANCE | MMS19 | PNKP | RBBP8 | TOPBP1 |
| ALKBH2 | CHAF1A | FANCF | MNAT1 | POLB | RDM1 | TP53 |
| ALKBH3 | CHEK1 | FANCG | MPG | POLD1 | RECQL | TP53BP1 |
| APEX1 | CHEK2 | FANCI | MRE11A | POLE | RECQL4 | TREX1 |
| APEX2 | CLK2 | FANCL | MSH2 | POLG | RECQL5 | TREX2 |
| APLF | CLSPN | FANCM | MSH3 | POLH | REV1 | UBE2A |
| APTX | DCLRE1A | FBXO18 | MSH4 | POLI | REV3L | UBE2B |
| ATM | DCLRE1B | FEN1 | MSH5 | POLK | RIF1 | UBE2I |
| ATR | DCLRE1C | GADD45A | MSH6 | POLL | RNF168 | UBE2N |
| ATRIP | DDB1 | GEN1 | MUS81 | POLM | RNF4 | UBE2V2 |
| BARD1 | DDB2 | GTF2H1 | MUTYH | POLN | RNF8 | UNG |
| BLM | DMC1 | GTF2H2 | NABP2 | POLQ | RPA1 | USP1 |
| BRCA1 | DUT | GTF2H3 | NBN | PRKDC | RPA2 | UVSSA |
| BRCA2 | E2F1 | GTF2H4 | NEIL1 | PRPF19 | RPA3 | WDR48 |
| BRIP1 | EME1 | GTF2H5 | NEIL2 | PTEN | RPA4 | WRN |
| C17orf70 | EME2 | H2AFX | NEIL3 | RAD1 | RRM2B | XAB2 |
| C19orf40 | ENDOV | HELQ | NHEJ1 | RAD17 | SETMAR | XPA |
| CCNB1 | ERCC1 | HLTF | NTHL1 | RAD18 | SHFM1 | XPC |
| CCND1 | ERCC2 | HUS1 | NUDT1 | RAD23A | SHPRH | XRCC1 |
| CCNE1 | ERCC3 | LIG1 | OGG1 | RAD23B | SLX1A | XRCC2 |
| CCNH | ERCC4 | LIG3 | PALB2 | RAD50 | SLX1B | XRCC3 |
| CDC25A | ERCC5 | LIG4 | PARP1 | RAD51 | SLX4 | XRCC4 |
| CDC25C | ERCC6 | MAD2L2 | PARP2 | RAD51B | SMUG1 | XRCC5 |
| CDH1 | ERCC8 | MBD4 | PARP3 | RAD51C | SPO11 | XRCC6 |
| CDK1 | EXO1 | MDC1 | PCNA | RAD51D | SPRTN | |
| CDK2 | FAN1 | MDM2 | PER1 | RAD52 | STK11 | |
| CDK4 | FANCA | MDM4 | PER2 | RAD54B | TDG | |
| CDK7 | FANCB | MGMT | PMS1 | RAD54L | TDP1 | |
| CDKN1A | FANCC | MLH1 | PMS2 | RAD9A | TDP2 | |
| CDKN1B | FANCD2 | MLH3 | PMS2P3 | RB1 | TOP2A | |

### Statistical Analysis

### Software and Libraries

All statistical analyses were applied using Python v3.8 [25]. Data in VCF format was converted using PyVCF v0.6.7 [26] and processed using pandas v1.3.0 [27], SciPy v1.4.1 [28], NumPy v1.18.3 [29], IPython v7.14 [30] and Scikits.bootstrap v1.1 [31]. Survival analysis for Cox's proportional hazard (PH) model and Kaplan-Meier estimates were performed using the Lifelines v0.25 package [32]. Tables and graphs were output using Matplolib v3.3.4 [33], to_precision [34] and Maftools v2.6.5 [35].

### Multifactor Cox Regression

Analyses were performed on the combined post-QC dataset (Table 13) and a subset of patients with high Gleason score tumours, with models stratified by study to compensate for differing baseline hazards. Gene-set predictors of the Cox Proportional Hazard (PH) model were generated by recording the presence of any gene with predicted-deleterious mutations in the selected gene-sets across all samples. Pathologic T-stage had a baseline of stage 1-2, and a second group for stage 3-4. Clinical T-stage was used for patients receiving radiotherapy (RT). Pre-operative PSA and age at time of surgery were continuous variables. Gleason score had a baseline of ≤3+4 (Gleason grade groups 1-2), and a group for ≥4+3 (Gleason grade group 3-5). Time was measured from radical treatment until BCR, which for samples with radical prostatectomy (RP) was defined as two consecutive post-RP PSA measurements of >0.2 ng/ml on the last known follow-up date [36]. For the 72 Canadian samples with RT, BCR was defined as a rise in PSA concentration of more than 2.0 ng/ml above the nadir, backdated to first PSA>0.2 ng/ml if PSA continues to rise [37]. A sensitivity analysis on a subset that excluded RT samples was performed, which did not affect the significant risk-elevating gene-sets observed (Table 13).

TABLE 13
Multifactor Cox model results for predicted-deleterious mutations
in 778 out of 850 germline samples (excluding patients treated
with radiotherapy), grouped into 52 gene-sets. Shown are p-
values and hazard ratios of all gene-sets as well as clinical
variables reported at time of biochemical recurrence (BCR)
or last check-up, impacting the predicted time until BCR

| | HR | p- |
|---|---|---|
| | (95% CI) | value |
| Gleason (≥4 + 3:<4 + 3) | 2.38 (1.72-3.31) | 2.13e−7 |
| Stage (T3-T4:T1-T2) | 1.99 (1.46-2.70) | 1.18e−5 |
| PI3K/AKT/mTOR | 1.60 (1.09-2.36) | 0.0177 |
| signalling | | |
| Inflammatory response | 1.41 (1.03-1.93) | 0.0321 |
| KRAS signalling (up) | 1.39 (1.02-1.88) | 0.0346 |
| Myc targets v2 | 1.28 (0.862-1.91) | 0.219 |
| p53 pathway | 1.26 (0.907-1.76) | 0.166 |
| DRG | 1.26 (0.946-1.67) | 0.115 |
| Age | 1.22 (0.929-1.60) | 0.152 |
| Fatty acid metabolism | 1.22 (0.896-1.65) | 0.209 |
| G2-M checkpoint | 1.18 (0.864-1.61) | 0.298 |
| IL-6/JAK/STAT3 | 1.11 (0.708-1.73) | 0.657 |
| signalling | | |
| Mitotic spindle | 1.10 (0.831-1.45) | 0.514 |
| Preop_PSA | 1.03 (1.01-1.06) | 0.0117 |
| UV response (dn) | 0.713 (0.499-1.02) | 0.0635 |
| Cholesterol homeostasis | 0.502 (0.276-0.915) | 0.0244 |

Variables included in the final models were selected by performing Cox regression with penalization based on the least absolute shrinkage and selection operator (LASSO) [38]. The optimal penalty factor (lambda) was determined as within 1 standard error of the optimum from the mean of 100 ten-fold cross-validation models. Only features with a non-zero coefficient were retained. The final prediction models were then built using Cox regression without penalization.

### Univariate Cox Regression

Each gene-set was modelled individually along with clinical covariates (pre-op PSA, pathologic T-stage, Gleason score, age), and p-values were adjusted for multiple testing using False Discovery Rate (FDR).

### Validation

Harmonised variant filtering was performed for predicted-deleterious mutations on germline PrCa samples from The Cancer Genome Atlas (TCGA) PRAD project. From the original 500 TCGA PRAD samples, any samples from contributing institutions with <15 samples were excluded, and models were stratified by institution, resulting in 383 samples used in the analysis. Of those, 233 were included in the high-Gleason subset analysis. The germline variants were applied to the predictors selected from the Cox model built using the combined PPCG samples, to compare the hazard ratios (HR) in both sets.

### Kaplan-Meier Analysis

A KM-plot measuring time to BCR in the event of relapse was used to visualise the impact of mutations within significant gene-sets on risk of BCR. This was applied separately to the whole dataset and high-Gleason subset, and reported alongside log-rank test p-values.

A combined analysis was performed, considering mutations in any of the gene-sets significant for the corresponding analyses, and subdivided to ascertain potential additive effects upon a patient's time to relapse.

### Bootstrapping Calidation

To test model robustness, new datasets were produced of the same sample size by randomly choosing samples with replacement, without stratification, and building a Cox regression model from the resulting dataset. This was repeated 1000 times to derive a distribution of coefficients. p-values were computed for each predictor as a percentage of the iterations where the coefficient was in a different direction than expected.

### Results

Germline WGS data was analyzed from 850 patients across five studies in the PPCG consortium (Table 8 and Table 9) for germline predictors of PrCa progression measured by BCR after radical treatment. This analysis was restricted to variants within protein-coding transcript sequences, resulting in 15,822 rare variants identified as deleterious or likely deleterious, jointly categorized as predicted-deleterious (PD). No individual variants or genes demonstrated significant association with time to BCR (Cox regression analysis; p-values >0.05), although the available sample size of 850 cases is underpowered for such analysis. Therefore, focus was on finding gene-sets or pathways with significant associations, to identify potential biological mechanisms linked with progression. To this end, whether there was at least one predicted-deleterious germline alteration in 52 gene-sets was determined, including 50 “Hallmark” gene-sets from the MsigDB database [24], containing over 4000 genes with sets varying in size from 30-200, the DRG panel containing 175 DNA repair genes [16], and the extended BROCA gene panel containing 65 genes [17].

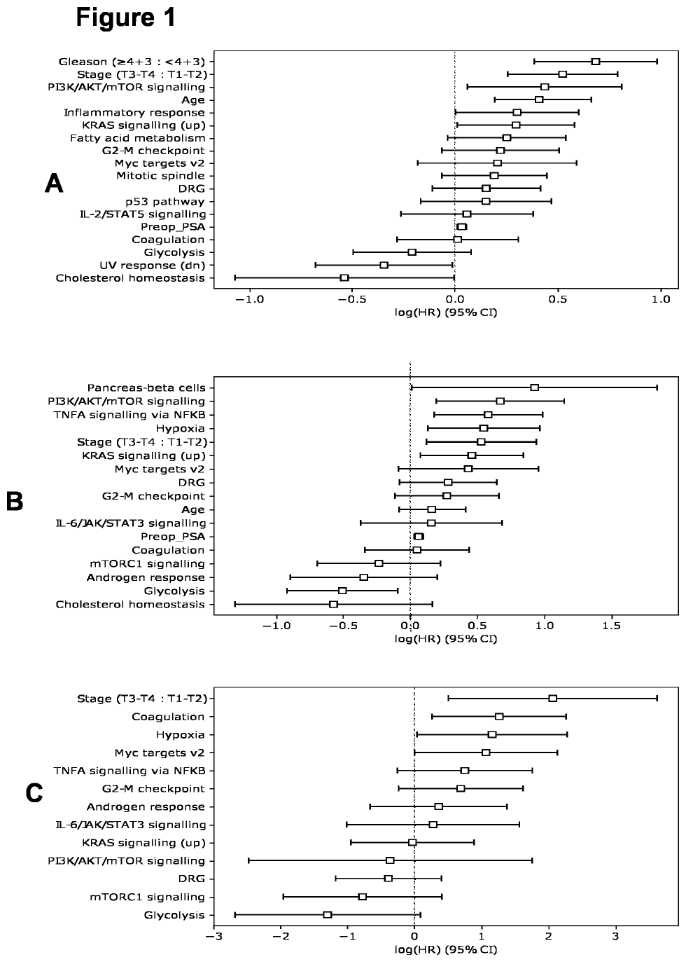

After variable selection by LASSO, the optimal model for predicting time to BCR contained fourteen gene-sets, three of which were significantly associated with time to BCR (Cox PH model for all samples; p-value threshold <0.05; Table 14 and FIG. 1a).

TABLE 14
Multifactor Cox model results for predicted-deleterious mutations in 850 germline
samples, grouped into 52 gene-sets. Shown are p-values and hazard ratios of
all gene-sets as well as clinical variables reported at time of biochemical
recurrence (BCR) or last check-up, impacting the predicted time until BCR.

| | HR | | Bootstrap HR | Bootstrap |
|---|---|---|---|---|
| | (95% CI) | p-value | (95% CI) | p-value |
| Gleason (≥4 + 3:< 4 + 3) | 1.98 (1.47-2.67) | 7.03 × 10⁻⁶ | 2.01 (1.99-2.04) | 0.00 |
| Stage (T3-T4:T1-T2) | 1.69 (1.29-2.21) | 1.32 × 10⁻⁴ | 1.75 (1.74-1.77) | 0.00 |
| PI3K/AKT/mTOR | 1.55 (1.06-2.25) | 0.0226 | 1.58 (1.56-1.60) | 0.0120 |
| signalling | | | | |
| Age | 1.53 (1.20-1.96) | 7.16 × 10⁻⁴ | 1.03 (1.03-1.03) | 1.00 × 10⁻³ |
| Inflammatory response | 1.35 (1.00-1.82) | 0.0483 | 1.37 (1.35-1.38) | 0.0280 |
| KRAS signalling (up) | 1.35 (1.01-1.79) | 0.0413 | 1.37 (1.36-1.38) | 0.0200 |
| Fatty acid metabolism | 1.29 (0.96-1.71) | 0.0868 | 1.32 (1.30-1.33) | 0.0400 |
| G2-M checkpoint | 1.25 (0.94-1.66) | 0.130 | 1.27 (1.26-1.28) | 0.0740 |
| Myc targets v2 | 1.23 (0.84-1.81) | 0.30 | 1.26 (1.24-1.27) | 0.155 |
| Mitotic spindle | 1.21 (0.94-1.56) | 0.142 | 1.22 (1.21-1.23) | 0.100 |
| DRG | 1.16 (0.90-1.51) | 0.26 | 1.18 (1.17-1.19) | 0.151 |
| p53 pathway | 1.16 (0.85-1.60) | 0.35 | 1.18 (1.16-1.19) | 0.21 |
| IL-2/STAT5 signalling | 1.06 (0.77-1.46) | 0.72 | 1.07 (1.06-1.09) | 0.38 |
| Preop_PSA | 1.04 (1.01-1.06) | 5.91 × 10⁻³ | 1.01 (1.01-1.01) | 4.00 × 10⁻³ |
| Coagulation | 1.01 (0.76-1.36) | 0.928 | 1.01 (1.00-1.02) | 0.51 |
| Glycolysis | 0.81 (0.61-1.08) | 0.155 | 0.82 (0.81-0.82) | 0.0800 |
| UV response (dn) | 0.71 (0.51-0.99) | 0.0418 | 0.72 (0.71-0.73) | 0.0380 |
| Cholesterol | 0.58 (0.34-1.00) | 0.0483 | 0.59 (0.58-0.60) | 0.0130 |
| homeostasis | | | | |

Clinical variables at the time of radical treatment (pre-op PSA, pathological T-stage, age and Gleason score) were added to the model as covariates. The significant risk-elevating Hallmarks were PI3K/AKT/mTOR (HR=1.55; 1.06-2.25 95% Cl; p=0.0226), Inflammatory response (HR=1.35; 1.00-1.82 95% Cl; p=0.0483) and KRAS signalling (up) (HR=1.35; 1.01-1.79 95% Cl; p=0.0413). These gene-sets are associated with shortened average time to BCR. The UV response (dn) (HR=0.71; 0.51-0.99 95% Cl; p=0.0418) and Cholesterol homeostasis (HR=0.58; 0.34-1.00 95% Cl; p=0.0483) gene-sets were borderline significantly protective. Applying this model to multiple bootstrap re-samplings showed that these results are robust, with all risk-elevating gene-sets HR>1 in >97% of resamples and p-values indicating the same coefficient direction.

The clinical covariates-only model built using all the samples determined that Gleason score, preop-PSA, age and pathological T-stage significantly associate with time to BCR (Cox PH; p-value threshold <0.05; Table 15). This model is significantly improved by the addition of the selected gene-sets (likelihood ratio test p=0.0477; c-index 0.68 vs 0.66).

TABLE 15
Multifactor Cox model results for clinical variables in
850 germline samples, impacting the predicted time until
biochemical recurrence. Gleason and T-stage were reported
at time of biochemical recurrence or last follow-up, while
age and PSA were reported at time of surgery.

| | HR (95% CI) | p-value |
|---|---|---|
| Gleason (≥4 + 3:<4 + 3) | 1.99 (1.49-2.66) | 2.81 × 10⁻⁶ |
| Stage (T3-T4:T1-T2) | 1.71 (1.31-2.24) | 7.70 × 10⁻⁵ |
| Age | 1.47 (1.15-1.87) | 2.01 × 10⁻³ |
| Preop_PSA | 1.04 (1.01-1.06) | 7.81 × 10⁻³ |

Within the PPCG set, patients presenting with higher-grade localised PrCa (a subset of 336 patients where Gleason score was 4+3 or higher; Gleason grade group 3-5) had a higher proportion of BCR events (50.2% compared to 33.5% for all samples; Table 8). An optimal multifactor Cox regression model was developed (Cox PH; p-value threshold <0.05; Table 16 and FIG. 1b) for this subset of high-Gleason samples with poorer prognosis disease. After feature selection by LASSO, we identified five significant risk-elevating gene-sets: Pancreas-beta cells (HR=2.52; 1.01-6.29 95% Cl; p=0.0470), PI3K/AKT/mTOR signalling (HR=1.95; 1.21-3.15 95% Cl; p=5.91×0⁻³), TNFA signalling via NFKB (HR=1.79; 1.19-2.6895% Cl; p=4.85×10⁻³), Hypoxia (HR=1.73; 1.14-2.6395% Cl; p=0.0101) and KRAS signalling (up) (HR=1.58; 1.08-2.32 95% Cl; p=0.0189). P3K/AKT/mTOR has a higher HR and lower p-value than in the all samples model. The Glycolysis gene-set shows here as significantly protective (HR=0.60; 0.40-0.91 95% Cl; p=0.0166). The bootstrap re-samplings for the significant gene-sets have the same coefficient direction in >96% of resamples.

TABLE 16
Multifactor Cox model results for predicted-deleterious mutations in 336 high-Gleason
germline samples, grouped into 52 gene-sets. Shown are p-values and hazard ratios
of all gene-sets impacting the predicted time until biochemical recurrence.

| | HR | | Bootstrap HR | Bootstrap |
|---|---|---|---|---|
| | (95% CI) | p-value | (95% CI) | p-value |
| Pancreas-beta cells | 2.52 (1.01-6.29) | 0.0470 | 3.58 (3.43-3.73) | 0.0340 |
| PI3K/AKT/mTOR | 1.95 (1.21-3.15) | 5.91 × 10⁻³ | 2.13 (2.09-2.17) | 7.00 × 10⁻³ |
| signalling | | | | |
| TNFA signalling | 1.79 (1.19-2.68) | 4.85 × 10⁻³ | 1.86 (1.83-1.89) | 5.00 × 10⁻³ |
| via NFKB | | | | |
| Hypoxia | 1.73 (1.14-2.63) | 0.0101 | 1.82 (1.79-1.85) | 0.0110 |
| Stage (T3-T4:T1-T2) | 1.70 (1.13-2.56) | 0.0115 | 1.86 (1.84-1.89) | 3.00 × 10⁻³ |
| KRAS signalling (up) | 1.58 (1.08-2.32) | 0.0189 | 1.65 (1.63-1.67) | 0.0160 |
| Myc targets v2 | 1.54 (0.92-2.60) | 0.104 | 1.60 (1.57-1.63) | 0.0810 |
| DRG | 1.33 (0.92-1.91) | 0.128 | 1.38 (1.36-1.39) | 0.0710 |
| G2-M checkpoint | 1.31 (0.89-1.93) | 0.167 | 1.41 (1.39-1.43) | 0.0920 |
| Age | 1.17 (0.90-1.52) | 0.24 | 1.01 (1.01-1.01) | 0.170 |
| IL-6/JAK/STAT3 | 1.17 (0.69-1.98) | 0.56 | 1.22 (1.19-1.24) | 0.31 |
| signalling | | | | |
| Preop_PSA | 1.06 (1.00-1.11) | 0.0391 | 1.00 (1.00-1.00) | 0.0280 |
| Coagulation | 1.05 (0.71-1.55) | 0.80 | 1.08 (1.06-1.09) | 0.41 |
| mTORC1 signalling | 0.79 (0.50-1.25) | 0.32 | 0.80 (0.79-0.82) | 0.172 |
| Androgen response | 0.71 (0.41-1.22) | 0.22 | 0.73 (0.72-0.74) | 0.121 |
| Glycolysis | 0.60 (0.40-0.91) | 0.0166 | 0.61 (0.60-0.62) | 0.0120 |
| Cholesterol | 0.564 (0.270-1.18) | 0.128 | 0.586 (0.571-0.602) | 0.0630 |
| homeostasis | | | | |

Examining each gene-set in individual univariate models with all samples, none had a significant association with outcome after multiple testing correction (FOR; p-value threshold <0.1; Table 17).

TABLE 17
Multifactor Cox model results for predicted-deleterious mutations in 383 The Cancer
Genome Atlas (TCGA) germline samples, stratified by location and grouped into 52 gene-
sets. Shown are p-values and hazard ratios of the same predictors identified by the
Pan Prostate Cancer Group (PPCG) Cox model (cholesterol homeostasis was removed as
samples have no mutations in this gene-set, which caused convergence errors).

| | HR | | Bootstrap HR | Bootstrap |
|---|---|---|---|---|
| | (95% CI) | p-value | (95% CI) | p-value |
| Myc targets v2 | 4.46 (1.73-11.5) | 1.99 × 10⁻³ | 6.43 | 6.00 × 10⁻³ |
| | | | (6.17-6.72) | |
| Coagulation | 3.49 (1.47-8.30) | 4.64 × 10⁻³ | 5.42 | 0.0110 |
| | | | (5.15-5.72) | |
| Gleason (≥4 + 3:<4 + 3) | 2.98 (1.06-8.33) | 0.0377 | 1.07 × 10⁶ | 6.00 × 10⁻³ |
| | | | (4.91 × 10⁵-2.08 × 10⁶) | |
| Stage (T3-T4:T1-T2) | 2.89 (1.01-8.28) | 0.0484 | 7.22 × 10¹² | 0.0400 |
| | | | (1.41 × 10⁶-4.33 × 10¹³) | |
| G2-M checkpoint | 2.19 (0.910-5.25) | 0.0805 | 3.08 | 0.0540 |
| | | | (2.94-3.26) | |
| Inflammatory response | 1.71 (0.615-4.77) | 0.303 | 2.12 | 0.212 |
| | | | (2.03-2.24) | |
| Fatty acid metabolism | 1.44 (0.496-4.18) | 0.503 | 1.77 | 0.265 |
| | | | (1.69-1.84) | |
| KRAS signalling (up) | 1.16 (0.531-2.54) | 0.706 | 1.29 | 0.402 |
| | | | (1.25-1.34) | |
| p53 pathway | 0.833 (0.306-2.26) | 0.720 | 0.912 | 0.355 |
| | | | (0.878-0.949) | |
| DRG | 0.814 (0.410-1.62) | 0.557 | 0.878 | 0.295 |
| | | | (0.853-0.905) | |
| Mitotic spindle | 0.727 (0.297-1.78) | 0.486 | 0.762 | 0.224 |
| | | | (0.736-0.791) | |
| PI3K/AKT/mTOR | 0.704 (0.0902-5.49) | 0.737 | 0.924 | 0.354 |
| signalling | | | (0.849-1.01) | |
| IL-2/STAT5 | 0.654 (0.263-1.62) | 0.360 | 0.806 | 0.254 |
| signalling | | | (0.772-0.845) | |
| UV response (dn) | 0.391 (0.104-1.47) | 0.165 | 0.518 | 0.131 |
| | | | (0.487-0.561) | |
| Glycolysis | 0.366 (0.104-1.29) | 0.117 | 0.464 | 0.0870 |
| | | | (0.441-0.491) | |

PI3KAKT/mTOR signalling (q=2.143), KRAS signalling (up) (q=0.203) and TNFA signalling via NKFB (q=0.157) had p-values close to the significance threshold, and achieve the threshold of significance in the high-Gleason subset (Table 17). In the high-Gleason subset, performing a log-rank test on each gene-set revealed four gene-sets that had a significant association with time to BCR: TNFA signalling via NFKB (p=0.0272), P3KAKT/mTOR signalling (p=0.0248), KRAS signalling (up) (p=0.0132) and Pancreas-beta cells (p=0.0233). In the multifactor high-Gleason Cox model these four gene-sets are also statistically significant (Table 17), alongside Hypoxia. Applying the all sample Cox multifactormodel to the TOGA validation set results in two significant gene-set predictors that are not reflected in the PPCG data: Myc targets v2 (HR=4.46; 1.73-11.5 95% Cl; p=1.99×10⁻³) and Coagulation (HR=3.49; 1.47-8.30 95% Cl; p=4.64×10⁻³) (Cox PH; p-value threshold <0.05;

Performing the same high-Gleason filtering on TOGA samples and applying that set to the high-Gleason PPCG model identifies three significant risk-elevating predictors: Myc targets v2 (HR=2.90; 1.00-8.40 95% Cl; p=0.0492) and Coagulation (HR=3.53; 1.30-9.59 95% Cl; p=0.0135), and additionally Hypoxia (HR=3.18; 1.04-9.74 95% Cl; p=0.0425) (Cox PH; p-value threshold <0.05; Table 18 and FIG. 1c).

TABLE 18
Multifactor Cox model results for predicted-deleterious mutations in 233 high-Gleason
The Cancer Genome Atlas (TCGA) germline samples, stratified by location and grouped
into 52 gene-sets. Shown are p-values and hazard ratios of the same predictors
identified by the Pan Prostate Cancer Group (PPCG) Cox model (pancreas-beta cells
and cholesterol homeostasis were removed as most samples had a mutation or had
no mutation in the gene-set respectively, which caused convergence errors).

| | HR | | Bootstrap HR | Bootstrap |
|---|---|---|---|---|
| | (95% CI) | p-value | (95% CI) | p-value |
| Stage (T3-T4:T1-T2) | 7.85 (1.65-37.3) | 9.55 × 10⁻³ | 6.24 × 10¹² | 1.00 × 10⁻³ |
| | | | (1.73 × 10⁸-3.73 × 10¹³) | |
| Coagulation | 3.53 (1.30-9.59) | 0.0135 | 11.3 (7.47-28.5) | 0.0220 |
| Hypoxia | 3.18 (1.04-9.74) | 0.0425 | 7.88 × 10⁶ | 0.0970 |
| | | | (1.14 × 10⁶-3.40 × 10⁷) | |
| Myc targets v2 | 2.90 (1.00-8.40) | 0.0492 | 5.63 (5.29-6.07) | 0.0440 |
| TNFA signalling | 2.12 (0.78-5.79) | 0.143 | 3.95 (3.51-4.97) | 0.110 |
| via NFKB | | | | |
| G2-M checkpoint | 2.00 (0.79-5.05) | 0.144 | 2.89 (2.75-3.11) | 0.102 |
| Androgen response | 1.43 (0.52-3.97) | 0.49 | 1.81 (1.70-2.00) | 0.32 |
| IL-6/JAK/STAT3 | 1.32 (0.36-4.77) | 0.68 | 2.86 × 10⁸ | 0.33 |
| signalling | | | (5.67-1.71 × 10⁹) | |
| KRAS signalling (up) | 0.97 (0.39-2.43) | 0.95 | 1.36 (1.29-1.46) | 0.51 |
| PI3K/AKT/mTOR | 0.70 (0.08-5.77) | 0.74 | 1.52 × 10⁶ | 0.31 |
| signalling | | | (0.972-7.60 × 10⁶) | |
| DRG | 0.68 (0.31-1.49) | 0.34 | 0.72 (0.70-0.75) | 0.181 |
| mTORC1 signalling | 0.46 (0.14-1.50) | 0.199 | 0.46 (0.43-0.49) | 0.0750 |
| Glycolysis | 0.27 (0.07-1.09) | 0.0667 | 0.34 (0.31-0.36) | 0.0470 |

The consistent significance, and same direction of coefficient of Hypoxia in patients with more advanced disease, is compelling evidence that germline variations in genes within this pathway contributes to clinical progression.

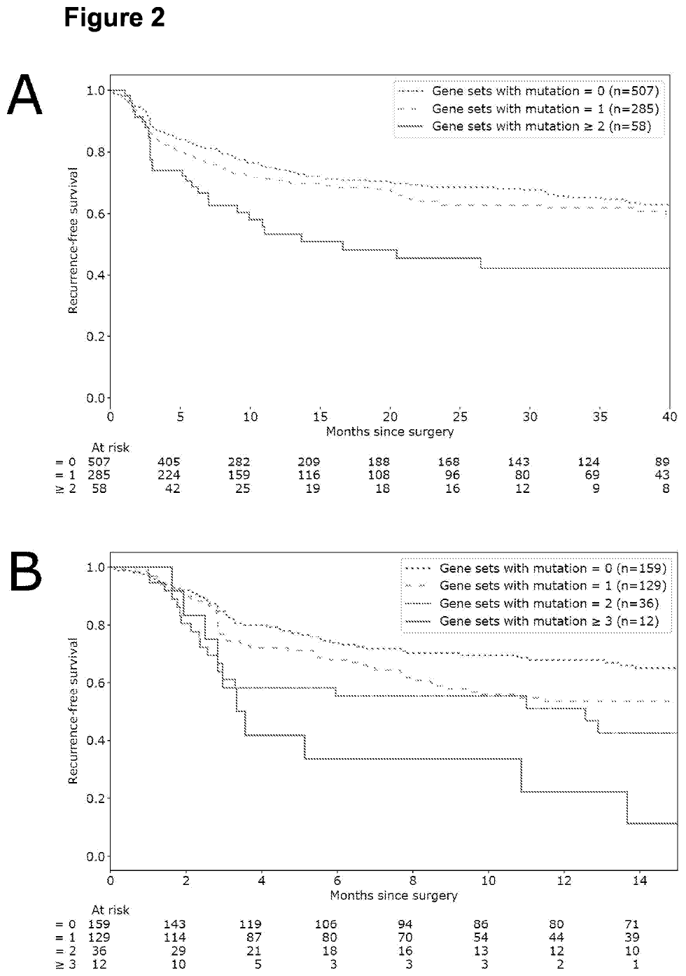

Kaplan-Meier plots were used to visualise the additive effect of mutations in the corresponding risk-elevating gene-sets for the all samples and high-Gleason sets (FIG. 2). In both plots there is shown a significant difference in survival when multiple gene-sets carry predicted-deleterious mutations. In the all samples analysis 285 of 850 patients had a mutation in one significant gene-set and 58 patients had mutations in two or more gene-sets, whilst in the high-Gleason subset analysis, 129 of 336 patients had a mutation in one significant gene-set, 36 patients had mutations in two gene-sets and 12 had mutations in three or more gene-sets, which was the clearest detrimental impact (FIG. 2B).

To search for individual genes mutated more frequently in patients with BCR, the odds ratio (OR) between the BCR positive and negative groups was calculated.

12 genes within the significant gene-sets for all samples (PIKFYVE, MYD88, CAB39, RPS6KA1, IRAK2, IL2RB, MSR1, ITGB8, PIK3R5, MMP10, HKDC1, RBM4) and 17 genes within the significant gene-sets in the high-Gleason subset (GAPDHS, GRHPR, PGM1, SELENBP1, NAGK, SLC6A6, PIKFYVE, MYD88, CAB39, RPS6KA1, DDX58, KYNU, NR4A1, DENND5A, MMP10, HKDC1, RBM4) had an OR at least 2-fold higher and a mutation count difference of 2 or more between samples with a mutation and BCR and those with a mutation and no BCR (Table 19).

TABLE 19
Odds Ratio results for the event of biochemical recurrence given predicted-
deleterious mutations in 850 germline samples. Results are filtered to include
only genes with OR > 2 and a difference between Has Mutation +
Has BCR vs Has Mutation + No BCR of at least two within the significant
all sample gene-sets: PI3K/AKT/mTOR signalling, KRAS signalling (up) and
Inflammatory response, and high-Gleason gene-sets: Hypoxia, PI3K/AKT/mTOR
signalling, TNFA signalling via NFKB and KRAS signalling (up). Pancreas-beta
cells is a significant high-Gleason gene-set, but has no genes with OR > 2.

| | | Has Mutation | No Mutation | |
|---|---|---|---|---|
| Gene-set | Gene | (Has BCR) | (Has BCR) | p |
| Hypoxia | GAPDHS | 8 (6) | 842 (279) | 0.0198 |
| | GRHPR | 2 (2) | 848 (283) | 0.112 |
| | PGM1 | 2 (2) | 848 (283) | 0.112 |
| | SELENBP1 | 2 (2) | 848 (283) | 0.112 |
| | NAGK | 2 (2) | 848 (283) | 0.112 |
| | SLC6A6 | 2 (2) | 848 (283) | 0.112 |
| PI3K/AKT/mTOR | PIKFYVE | 6 (5) | 844 (280) | 0.0180 |
| signalling | | | | |
| | MYD88 | 2 (2) | 848 (283) | 0.112 |
| | CAB39 | 2 (2) | 848 (283) | 0.112 |
| | RPS6KA1 | 2 (2) | 848 (283) | 0.112 |
| TNFA signalling | DDX58 | 3 (3) | 847 (282) | 0.0374 |
| via NFKB | | | | |
| | KYNU | 2 (2) | 848 (283) | 0.112 |
| | NR4A1 | 2 (2) | 848 (283) | 0.112 |
| | DENND5A | 2 (2) | 848 (283) | 0.112 |
| KRAS signalling (up) | MMP10 | 7 (5) | 843 (280) | 0.0457 |
| | HKDC1 | 6 (4) | 844 (281) | 0.0938 |
| | RBM4 | 2 (2) | 848 (283) | 0.112 |
| Inflammatory response | IRAK2 | 4(4) | 846 (281) | 0.0125 |
| | IL2RB | 3(3) | 847 (282) | 0.0374 |
| | MSR1 | 2(2) | 848 (283) | 0.112 |
| | ITGB8 | 2(2) | 848 (283) | 0.112 |
| | PIK3R5 | 2(2) | 848 (283) | 0.112 |

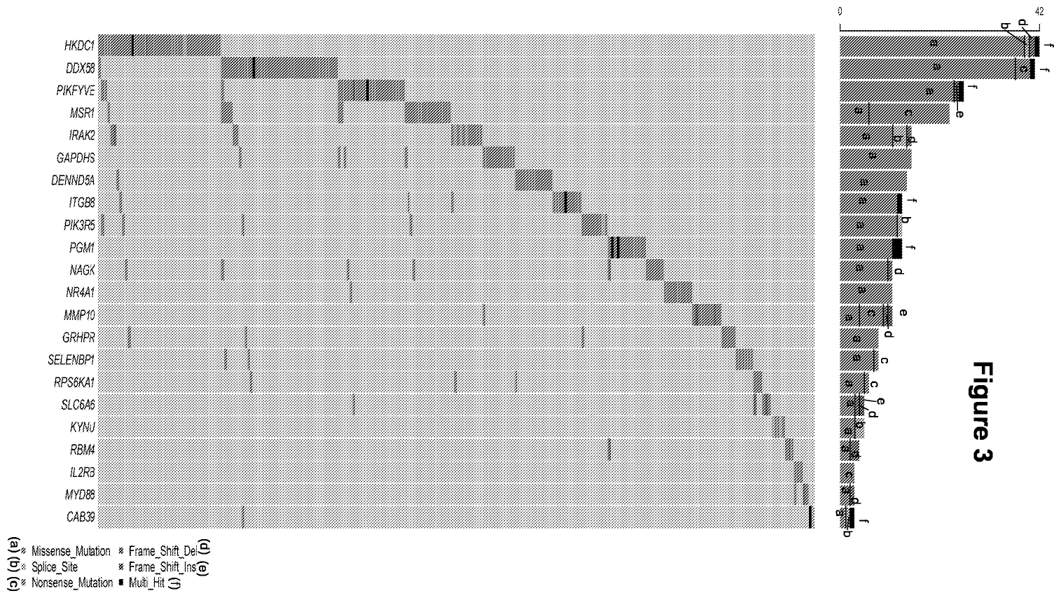

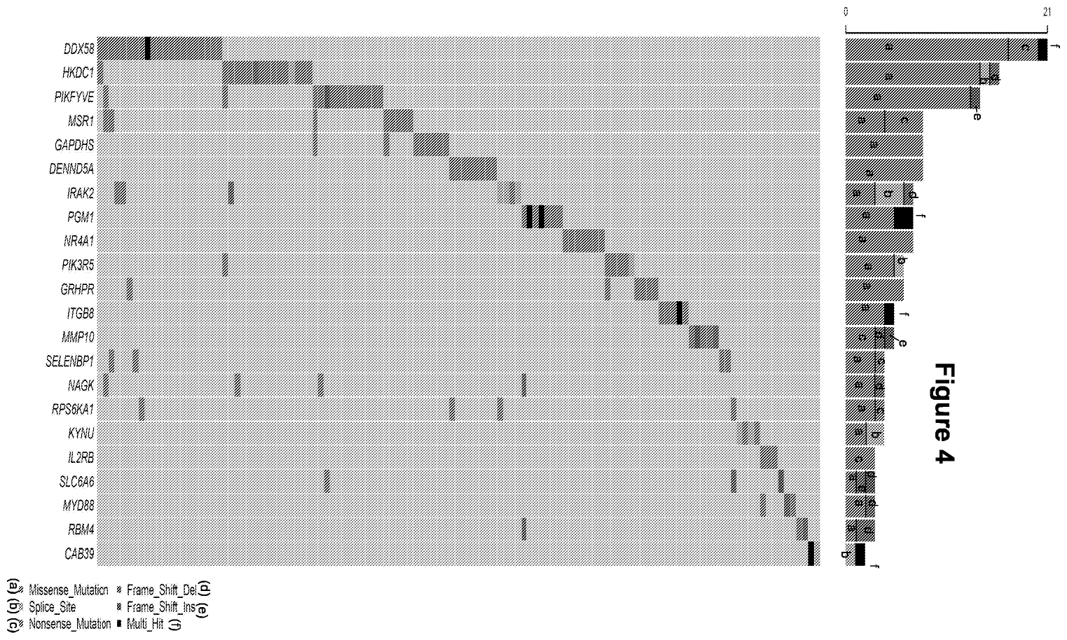

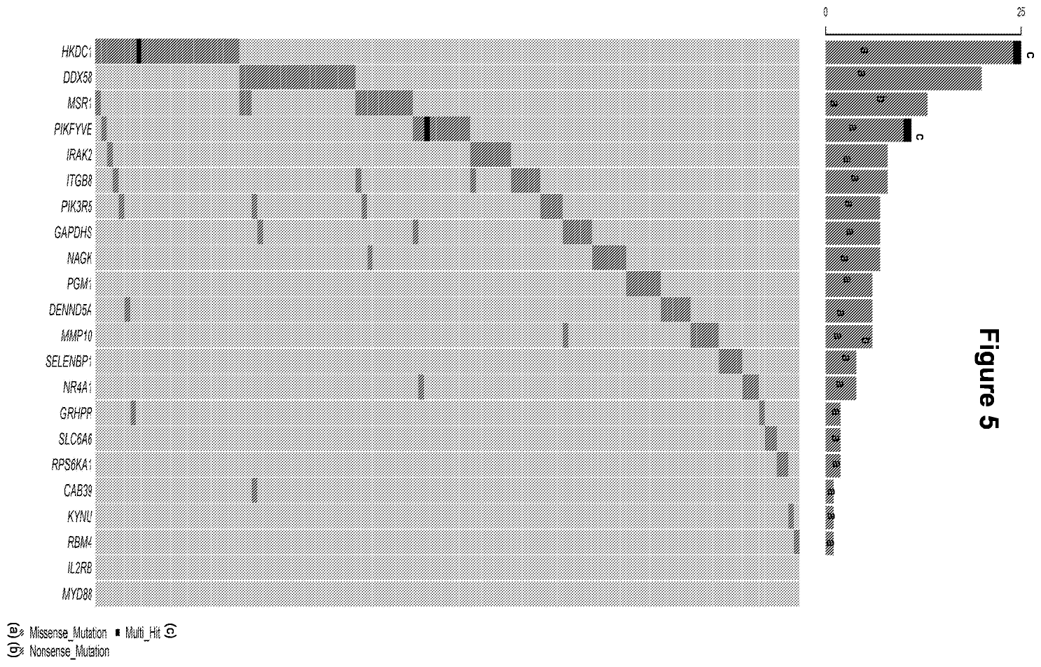

The overwhelming majority (92.7%) of the PD mutations identified in these combined 22 risk-elevating genes are missense variants (FIG. 3), although patients with BCR exhibited more non-missense variants (FIG. 4) compared with those without BCR (FIG. 5).

### DISCUSSION

The primary aim of genetic profiling of germline or tumour DNA is to aid clinical decisions, such as targeted screening of asymptomatic individuals and treatment options for cancer patients. Germline signatures in particular would have the advantage of helping to stratify patients in both pre- and post-operative settings. Follow-up strategies and decisions on further treatments could be aided by predicting which individuals are likely to develop prostate tumours, progress to clinically significant disease or relapse. This study is the first to evaluate association of rare germline mutations across the full exome as opposed to specific plausible candidate genes, and provides evidence that germline mutation status is predictive for BCR after radical treatment for PrCa. Our multifactor Cox model identified that rare predicted-deleterious variants in three Hallmark gene-sets are associated with time to BCR after radical treatment (PI3K/AKT/mTOR, KRAS signalling (up) and Inflammatory response), and five gene-sets associated with BCR in a subset of cases with more aggressive phenotype at diagnosis (PI3K/AKT/mTOR, KRAS signalling (up), Hypoxia, TNFA signalling via NFKB and Pancreas-beta cells). Importantly, it is also shown that these gene-sets remained an independent predictive biomarker of time to BCR, over and above the inclusion of clinical variables. It is further demonstrated that the Hypoxia gene-set replicated in an independent cohort of high-Gleason tumour cases from TCGA. These signatures could inform prognosis and clinical decision making.

Among the gene-sets associated with greater risk of BCR in PrCa patients, genes involved in PI3K/AKT/mTOR and KRAS signalling (up) remained significant across all PPCG samples as well as when restricted to patients with high-Gleason tumours. In somatic analyses, AKT expression and phosphorylation have previously been linked to risk of BCR after radical prostatectomy [39, 40] and poorer survival in patients with metastatic castrate-resistant PrCa [41]. Somatic loss of PTEN, a tumour suppressor that downregulates the AKT signalling pathway, is also associated with poorer prognosis PrCa [5] and disease recurrence [42, 43].

In the analysis of patients with high-Gleason tumours, the Hypoxia gene-set was established at statistical significance in the PPCG cohort and also replicated in the independent TCGA validation cohort. This provides strong evidence that germline mutations within this gene-set contribute to recurrence in patients with more aggressive disease. Hypoxia has previously been reported to contribute to progression when analysing tumour samples [44, 45], with a 28 gene mRNA signature for hypoxia demonstrated to predict BCR and metastases after radical prostatectomy or radiotherapy and provide independent prognostic value after adjustment for clinical features [46]. The results indicate for the first time that heritable mutations in genes upregulated in response to a low oxygen environment predispose PrCa patients towards greater likelihood of, and shorter time to, BCR.

A small number of additional gene-sets also achieved significance in a single analysis only (Inflammatory response in PPCG all samples, TNFA signalling via NFKB and Pancreas-beta cells in the PPCG high-Gleason subset, and Myc targets v2 and Coagulation in the TCGA validation cohort). Due to the less consistent selection of these gene-sets, the importance of these gene-sets in germline susceptibility towards BCR is less compelling; however they would nonetheless represent potential gene-sets of interest.

In this study, significantly shorter time to BCR among the individuals carrying mutations in >1 of the risk-increasing gene-sets is shown. 58 out of the 850 total patients having mutations in multiple of the three all samples gene-sets, and 48 out of the 336 patients having mutations in multiple of the five high-Gleason gene-sets identified through the multifactor analysis, compared to both non-carriers and individuals carrying mutations in a single gene-set only. This provides further support that mutations affecting multiple regulatory networks may co-operatively serve to negatively influence PrCa prognosis; and that for some men, intraprostatic features that determine an aggressive tumour environment may be predetermined in the germline. This has been suggested before, based on hypoxia associating with genetic instability and aggressive sub-pathologies as field defects in PrCa, and warrants further investigation [47].

This analysis included only coding variants with strong evidence for deleterious effect, excluding variants of uncertain significance, copy number alterations and structural variants. It may be necessary to integrate different data types, including expression and methylation data, to fully understand the mechanisms behind the findings. Although it is very encouraging that genes curated within PI3K/AKT/mTOR signalling and KRAS signalling (up) remained significant across both the PPCG all samples and high Gleason subset analyses, and the independent validation cohort confirmed evidence for genes curated as involved in Hypoxia, additional larger studies remain necessary to confirm these findings and disentangle which specific genes contribute towards increased risk of PrCa progression and invasiveness.

The findings have potentially important clinical implications. Germline DNA can be sequenced at an early stage of disease or even for healthy individuals which could enable prediction of PrCa progression close to, or even in advance of, the point of diagnosis. This would allow clinicians to stratify and differentiate patients that are more likely to relapse, putting them on a different clinical treatment pathway comprising more radical intervention or more frequent follow-up.

Prostate cancer patients with inherited mutations in specific genes demonstrate a greater likelihood of relapsing after initial radical treatment. In the future, we may be able to use genetic information to identify sooner which patients may require additional treatments, and in turn improve prognoses for these individuals.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

[1] Dell'Oglio P, Stabile A, Gandaglia G, et al. New surgical approaches for clinically high-risk or metastatic prostate cancer. Expert Rev Anticancer Ther. 2017; 17:1013-31.
[2] Vickers A J, Ulmert D, Sjoberg D D, et al. Strategy for detection of prostate cancer based on relation between prostate specific antigen at age 40-55 and long term risk of metastasis: case-control study. BMJ. 2013; 346:f2023.
[3] Saunders E J, Kote-Jarai Z, Eeles R A. Identification of Germline Genetic Variants that Increase Prostate Cancer Risk and Influence Development of Aggressive Disease. Cancers (Basel). 2021; 13.
[4] Hull G W, Rabbani F, Abbas F, Wheeler T M, Kattan M W, Scardino P T. Cancer control with radical prostatectomy alone in 1,000 consecutive patients. J Urol. 2002; 167:528-34.
[5] Bostrom P J, Bjartell A S, Catto J W, et al. Genomic Predictors of Outcome in Prostate Cancer. Eur Urol. 2015; 68:1033-44.
[6] Li H, Durbin R. Fast and accurate long-read alignment with Burrows-Wheeler transform. Bioinformatics. 2010; 26:589-95.
[7] Cooper C S, Eeles R, Wedge D C, et al. Analysis of the genetic phylogeny of multifocal prostate cancer identifies multiple independent clonal expansions in neoplastic and morphologically normal prostate tissue. Nat Genet. 2015; 47:367-72.
[8] McKenna A, Hanna M, Banks E, et al. The Genome Analysis Toolkit: a MapReduce framework for analyzing next-generation DNA sequencing data. Genome Res. 2010; 20:1297-303.
[9] DePristo M A, Banks E, Poplin R, et al. A framework for variation discovery and genotyping using next-generation DNA sequencing data. Nat Genet. 2011; 43:491-8.
[10] Van der Auwera GAOC, B. D. Genomics in the Cloud: Using Docker, GATK, and WDL in Terra (1st Edition). O'Reilly Media. 2020.
[11] Garrison E.; Marth G. Haplotype-based variant detection from short-read sequencing. arXiv preprint arXiv:12073907 [q-bioGN]. 2012.
[12] Gerhauser C, Favero F, Risch T, et al. Molecular Evolution of Early-Onset Prostate Cancer Identifies Molecular Risk Markers and Clinical Trajectories. Cancer Cell. 2018; 34:996-1011 e8.
[13] Tan A, Abecasis G R, Kang H M. Unified Representation of Genetic Variants. Bioinformatics. 2015; 31:2202-4.
[14] Frankish A, Diekhans M, Ferreira A M, et al. GENCODE reference annotation for the human and mouse genomes. Nucleic Acids Res. 2019; 47:D766-D73.
[15] Anderson C A, Pettersson F H, Clarke G M, Cardon L R, Morris A P, Zondervan K T. Data quality control in genetic case-control association studies. Nat Protoc. 2010; 5:1564-73.
[16] Leongamornlert D A, Saunders E J, Wakerell S, et al. Germline DNA Repair Gene Mutations in Young-onset Prostate Cancer Cases in the UK: Evidence for a More Extensive Genetic Panel. Eur Urol. 2019; 76:329-37.
[17] Mijuskovic M, Saunders E J, Leongamornlert D A, et al. Rare germline variants in DNA repair genes and the angiogenesis pathway predispose prostate cancer patients to develop metastatic disease. BrJ Cancer. 2018; 119:96-104.
[18] Zheng X, Levine D, Shen J, Gogarten S M, Laurie C, Weir B S. A high-performance computing toolset for relatedness and principal component analysis of SNP data. Bioinformatics. 2012; 28:3326-8.
[19] Genomes Project C, Auton A, Brooks L D, et al. A global reference for human genetic variation. Nature. 2015; 526:68-74.
[20] Picard Tools. Broad Institute, GitHub repository (2021; v2238; http://broadinstitutegithubio/picard/).
[21] Jun G, Flickinger M, Hetrick K N, et al. Detecting and estimating contamination of human DNA samples in sequencing and array-based genotype data. Am J Hum Genet. 2012; 91:839-48.
[22] Karczewski K J, Francioli L C, Tiao G, et al. The mutational constraint spectrum quantified from 50 variation in 141,456 humans. Nature. 2020; 581:434-43.
[23] Kircher M, Witten D M, Jain P, O'Roak B J, Cooper G M, Shendure J. A general framework for estimating the relative pathogenicity of human genetic variants. Nat Genet. 2014; 46:310-5.
[24] Liberzon A, Birger C, Thorvaldsdottir H, Ghandi M, Mesirov J P, Tamayo P. The Molecular Signatures Database (MSigDB) hallmark gene set collection. Cell Syst. 2015; 1:417-25.
[25] Van Rossum G, Drake Jr F L. Python reference manual. Centrum voor Wiskunde en Informatica Amsterdam. 1995.
[26] Casbon J, Dougherty J. PyVCF. (https://pyvcfreadthedocsio/). 2011.
[27] Reback J, jbrockmendel, McKinney W, et al. pandas-dev/pandas: v1.3.0. Zenodo. https://doi.org/10.5281/zenodo.5060318. 2021.
[28] Virtanen P, Gommers R, Oliphant T E, et al. SciPy 1.0: Fundamental Algorithms for Scientific Computing in Python. Nature Methods. 2020; 17:261-72.
[29] Harris C R, Jarrod Millman K, van der Walt S J, et al. Array programming with NumPy. Nature. 2020; 585:357-62.
[30]Pérez F, Granger B E. IPython: A System for Interactive Scientific Computing. Computing in Science & Engineering. 2007; 9:21-9.
[31] Evans C, Paolo F, Natsuo Kishimoto P, et al. cgevans/scikits-bootstrap: v1.1.0-pre.1. Zenodo. https://doi.org/10.5281/zenodo.5095250. 2021.
[32] Davidson-Pilon C, Kalderstam J, Jacobson N, et al. CamDavidsonPilon/Lifelines: v0.25.0. Zenodo. https://doi.org/10.5281/zenodo.3962065. 2020.
[33] Hunter J D. Matplotlib: A 2D Graphics Environment. Computing in Science & Engineering. 2007; 9:90-5.
[34] Rusnack W, Moyer E, Taylor R. to_precision. [accessed on 16 Nov. 2021]; Available online: https://bitbucketorg/william rusnack/to-precision/src/master/. 2013.
[35] Mayakonda A, Lin D C, Assenov Y, Plass C, Koeffler H P. Maftools: efficient and comprehensive analysis of somatic variants in cancer. Genome Res. 2018; 28:1747-56.
[36] Budaus L, Schiffmann J, Graefen M, et al. Defining biochemical recurrence after radical prostatectomy and timing of early salvage radiotherapy: Informing the debate. Strahlenther Onkol. 2017; 193:692-9.
[37] Fraser M, Sabelnykova V Y, Yamaguchi T N, et al. Genomic hallmarks of localized, non-indolent prostate cancer. Nature. 2017; 541:359-64.
[38] Tibshirani R. Regression Shrinkage and Selection via the Lasso. J R Statist Soc B. 1996; 58:267-88.
[39] Bedolla R, Prihoda T J, Kreisberg J I, et al. Determining risk of biochemical recurrence in prostate cancer by immunohistochemical detection of PTEN expression and Akt activation. Clin Cancer Res. 2007; 13:3860-7.
[40] Torrealba N, Rodriguez-Berriguete G, Fraile B, et al. PI3K pathway and Bcl-2 family. Clinicopathological features in prostate cancer. Aging Male. 2018; 21:211-22.
[41] McCall P, Gemmell L K, Mukherjee R, Bartlett J M, Edwards J. Phosphorylation of the androgen receptor is associated with reduced survival in hormone-refractory prostate cancer patients. Br J Cancer. 2008; 98:1094-101.
[42] Geybels M S, Fang M, Wright J L, et al. PTEN loss is associated with prostate cancer recurrence and alterations in tumor DNA methylation profiles. Oncotarget. 2017; 8:84338-48.
[43] Chaux A, Peskoe S B, Gonzalez-Roibon N, et al. Loss of PTEN expression is associated with increased risk of recurrence after prostatectomy for clinically localized prostate cancer. Modern Pathol. 2012; 25:1543-9.
[44] Bhandari V, Hoey C, Liu L Y, et al. Molecular landmarks of tumor hypoxia across cancer types. Nat Genet. 2019; 51:308-18.
[45] Lalonde E, Ishkanian A S, Sykes J, et al. Tumour genomic and microenvironmental heterogeneity for integrated prediction of 5-year biochemical recurrence of prostate cancer: a retrospective cohort study. Lancet Oncol. 2014; 15:1521-32.
[46] Yang L, Roberts D, Takhar M, et al. Development and Validation of a 28-gene Hypoxia-related Prognostic Signature for Localized Prostate Cancer. EBioMedicine. 2018; 31:182-9.
[47] Chua M L K, Lo W, Pintilie M, et al. A Prostate Cancer “Nimbosus”: Genomic Instability and SChLAP1 Dysregulation Underpin Aggression of Intraductal and Cribriform Subpathologies. Eur Urol. 2017; 72:665-74.

1. A method of predicting a patient's prognosis of prostate cancer, the method comprising:
(a) providing a sample of the patient's germline genetic material;
(b) analysing the patient's germline genetic material;
(c) detecting at least one germline variant of at least one gene selected from at least one of:
genes up-regulated by activation of the PI3K/AKT/mTOR pathway;
genes defining inflammatory response;
genes up-regulated by KRAS activation;
genes up-regulated in response to low oxygen levels;
genes regulated by NF-kB in response to tumour necrosis factor (TNF); and/or
genes specifically up-regulated in pancreatic beta cells; or
at least one gene from Table 1;
wherein the prognosis of prostate cancer comprises a characteristic of relapse; and
wherein detection of the least one germline variant is predicative of the characteristic of relapse of the prostate cancer patient.

2. The method of claim 1, wherein the characteristic of relapse is time to biochemical relapse (BCR) and/or likelihood of BCR.

3. The method of claim 1, wherein the patient suffers from prostate cancer or is at risk of prostate cancer.

4. The method of claim 1, wherein the patient suffers from prostate cancer or has suffered from prostate cancer and has undergone radical therapy.

5. The method of claim 1, wherein the at least one variant comprises a predicted deleterious mutation.

6. The method of claim 5, wherein the predicted deleterious mutation comprises a protein-truncating mutation of an encoded protein, and/or
wherein the predicted-deleterious variant is a missense variant comprising a CADD PHRED score >30; and/or
wherein the protein-truncating mutation comprises one or more of a nonsense, a frameshift and/or a splice site variant.

7. The method of claim 1, wherein the at least one germline variant comprises a rare variant, and/or wherein the at least one germline variant comprises a minor allele frequency of less than 1%.

8. The method of claim 1, wherein the least one germline variant comprises a variant of at least one gene selected from at least one of:
the genes of Table 2 (M5923 HALLMARK_PI3K_AKT_MTOR_SIGNALING);
the genes of Table 3 (M5932 HALLMARK_INFLAMMATORY_RESPONSE);
the genes of Table 4 (M5953 HALLMARK_KRAS_SIGNALING_UP);
the genes of Table 5 (M5957 HALLMARK_PANCREAS_BETA_CELLS);
the genes of Table 6 (M5890 HALLMARK_TNFA_SIGNALING_VIA_NFKB); and/or
the genes of Table 7 (M5891 HALLMARK_HYPOXIA).

9. The method of claim 1, wherein the at least one germline variant comprises a variant of at least one of:
PIKFYVE, MYD88, CAB39, RPS6KA1, IRAK2, IL2RB, MSR1, ITGB8, PIK3R5, MMP10, HKDC1, RBM4, GAPDHS, GRHPR, PGM1, SELENBP1, NAGK, SLC6A6, DDX58, KYNU, NR4A1, and/or DENND5A; and/or
at least one of PIKFYVE, MYD88, CAB39, RPS6KA1, IRAK2, IL2RB, MSR1, ITGB8, PIK3R5, MMP10, HKDC1 and/or RBM4.

10. The method of claim 1, wherein detection of the least one germline variant is predicative of the patient's response to a treatment.

11. The method of claim 1, wherein the characteristic of relapse comprises time to BCR and the least one germline variant comprises a variant of at least one gene selected from:
the genes of Table 2 (M5923 HALLMARK_PI3K_AKT_MTOR_SIGNALING);
the genes of Table 3 (M5932 HALLMARK_INFLAMMATORY_RESPONSE); and/or
the genes of Table 4 (M5953 HALLMARK_KRAS_SIGNALING_UP).

12. The method of claim 1, wherein the patient has been diagnosed with a high-grade prostate cancer.

13. The method of claim 12, wherein the least one germline variant comprises a variant of at least one gene selected from:
the genes of Table 2 (M5923 HALLMARK_PI3K_AKT_MTOR_SIGNALING);
the genes of Table 4 (M5953 HALLMARK_KRAS_SIGNALING_UP);
the genes of Table 5 (M5957 HALLMARK_PANCREAS_BETA_CELLS);
the genes of Table 6 (M5890 HALLMARK_TNFA_SIGNALING_VIA_NFKB); and/or
the genes of Table 7 (M5891 HALLMARK_HYPOXIA).

14. The method of claim 12, wherein the least one germline variant comprises a variant of at least one of: GAPDHS, GRHPR, PGM1, SELENBP1, NAGK, SLC6A6, PIKFYVE, MYD88, CAB39, RPS6KA1, DDX58, KYNU, NR4A1, DENND5A, MMP10, HKDC1, and/or RBM4.

15. A method of determining a treatment regimen for a prostate cancer patient, the method comprising;
(a) providing a sample of the patient's germline genetic material;
(b) analysing the patient's germline genetic material;
(c) detecting at least one germline variant of at least one gene selected from at least one of;
genes up-regulated by activation of the PI3K/AKT/mTOR pathway;
genes defining inflammatory response;
genes up-regulated by KRAS activation;
genes up-regulated in response to low oxygen levels;
genes regulated by NF-kB in response to tumour necrosis factor (TNF); and/or
genes specifically up-regulated in pancreatic beta cells; or
at least one gene from Table 1;
(d) determining a treatment regimen based on the detection of the at least one germline variant.

16-18. (canceled)

19. A signature biomarker panel characteristic of time to biochemical relapse and/or likelihood of biochemical relapse for a prostate cancer patient, the panel comprising at least one germline variant of at least one gene selected from at least one of;
genes up-regulated by activation of the PI3K/AKT/mTOR pathway;
genes defining inflammatory response;
genes up-regulated by KRAS activation;
genes up-regulated in response to low oxygen levels;
genes regulated by NF-kB in response to tumour necrosis factor (TNF); and/or
genes specifically up-regulated in pancreatic beta cells; or
at least one gene from Table 1.

20. The signature biomarker panel claim 19, wherein the at least one variant comprises a predicted deleterious mutation.

21. The signature biomarker panel of claim 19, wherein the least one germline variant comprises a variant of at least one of:
PIKFYVE, MYD88, CAB39, RPS6KA1, IRAK2, IL2RB, MSR1, ITGB8, PIK3R5, MMP10, HKDC1, RBM4, GAPDHS, GRHPR, PGM1, SELENBP1, NAGK, SLC6A6, DDX58, KYNU, NR4A1, and/or DENND5A; and/or
at least one of PIKFYVE, MYD88, CAB39, RPS6KA1, IRAK2, IL2RB, MSR1, ITGB8, PIK3R5, MMP10, HKDC1, and/or RBM4.

22. The signature biomarker panel of claim 19, wherein the patient has been diagnosed with a high-grade prostate cancer and wherein the least one germline variant comprises a variant of at least one of: GAPDHS, GRHPR, PGM1, SELENBP1, NAGK, SLC6A6, PIKFYVE, MYD88, CAB39, RPS6KA1, DDX58, KYNU, NR4A1, DENND5A, MMP10, HKDC1, and/or RBM4.

23. The signature biomarker panel of claim 19, wherein the patient suffers from prostate cancer or is at risk of prostate cancer; and/or
wherein the patient suffers from prostate cancer or has suffered from prostate cancer and has undergone radical therapy.
